SEQUENCE LISTING (47 sequences)

SEQ ID NO: 1 - protein, 604 aa, synthetic construct
EWVKFAKPCREREDNSRRNPIAKITSDFQKNQKITYRISGVGIDQPPFGIFVVDPNNGDINITAIVDREETPSFLITCRALNALGQDVERPLILTVKILDVNDNPPIFSQTIFKGEIEENSASNSLVMILNATDADEPNHMNSKIAFKIVSQEPAGMSMFLISRNTGEVRTLTSSLDREQISSYHLVVSGADNDGTGLSTQCECSIKIKDVNDNFPVLRESQYSARIEENTLNAELLRFQVTDWDEEYTDNWLAVYFFTSGNEGNWFEIETDPRTNEGILKVVKALDYEQVQSMQFSIAVRNKAEFHQSVISQYRVQSTPVTIQVIDVREGISGGGGSGGSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGSGGGGSDYKDDDDKLPETGGHHHHHH

SEQ ID NO: 2 - protein, 595 aa, synthetic construct
EWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSDYKDDDDKLPETGGHHHHHH

SEQ ID NO: 3 - protein, 697 aa, synthetic construct
EWVKFAKPCREREDNSRRNPIAKITSDFQKNQKITYRISGVGIDQPPFGIFVVDPNNGDINITAIVDREETPSFLITCRALNALGQDVERPLILTVKILDVNDNPPIFSQTIFKGEIEENSASNSLVMILNATDADEPNHMNSKIAFKIVSQEPAGMSMFLISRNTGEVRTLTSSLDREQISSYHLVVSGADNDGTGLSTQCECSIKIKDVNDNFPVLRESQYSARIEENTLNAELLRFQVTDWDEEYTDNWLAVYFFTSGNEGNWFEIETDPRTNEGILKVVKALDYEQVQSMQFSIAVRNKAEFHQSVISQYRVQSTPVTIQVIDVREGISFRPPSKTFTVQRGVSTNKLVGYILGTYQATDEDTGKAASSVRYVLGRNDGGLLVIDSKTAQIKFVKNIDRDSTFIVNKTISAEVLAIDENTGKTSTGTIYVEVPSFNENGSPTIKPSPPSKSPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKEFDYKDDDDKGSLPETGGHHHHHH

SEQ ID NO: 4 - protein, 701 aa, synthetic construct
EWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNGGGGSEPKSADKTHTSPPAPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGEFDYKDDDDKLPETGHHHHHH

SEQ ID NO: 5 - protein, 999 aa, Homo sapiens
MMGLFPRTTGALAIFVVVILVHGELRIETKGQYDEEEMTMQQAKRRQKREWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKDAVCSSSPSVVVSARTLNNRYTGPYTFALEDQPVKLPAVWSITTLNATSALLRAQEQIPPGVYHISLVLTDSQNNRCEMPRSLTLEVCQCDNRGICGTSYPTTSPGTRYGRPHSGRLGPAAIGLLLLGLLLLLLAPLLLLTCDCGAGSTGGVTGGFIPVPDGSEGTIHQWGIEGAHPEDKEITNICVPPVTANGADFMESSEVCTNTYARGTAVEGTSGMEMTTKLGAATESGGAAGFATGTVSGAASGFGAATGVGICSSGQSGTMRTRHSTGGTNKDYADGAISMNFLDSYFSQKAFACAEEDDGQEANDCLLIYDNEGADATGSPVGSVGCCSFIADDLDDSFLDSLGPKFKKLAEISLGVDGEGKEVQPPSKDSGYGIESCGHPIEVQQTGFVKCQTLSGSQGASALSTSGSVQPAVSIPDPLQHGNYLVTETYSASGSLVQPSTAGFDPLLTQNVIVTERVICPISSVPGNLAGPTQLRGSHTMLCTEDPCSRLI

SEQ ID NO: 6 - protein, 1049 aa, Homo sapiens
MDWSFFRVVAMLFIFLVVVEVNSEFRIQVRDYNTKNGTIKWHSIRRQKREWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNTKITTNTGRQESTSSTNYDTSTTSTDSSQVYSSEPGNGAKDLLSDNVHFGPAGIGLLIMGFLVLGLVPFLMICCDCGGAPRSAAGFEPVPECSDGAIHSWAVEGPQPEPRDITTVIPQIPPDNANIIECIDNSGVYTNEYGGREMQDLGGGERMTGFELTEGVKTSGMPEICQEYSGTLRRNSMRECREGGLNMNFMESYFCQKAYAYADEDEGRPSNDCLLIYDIEGVGSPAGSVGCCSFIGEDLDDSFLDTLGPKFKKLADISLGKESYPDLDPSWPPQSTEPVCLPQETEPVVSGHPPISPHFGTTTVISESTYPSGPGVLHPKPILDPLGYGNVTVTESYTTSDTLKPSVHVHDNRPASNVVVTERVVGPISGADLHGMLEMPDLRDGSNVIVTERVIAPSSSLPTSLTIHHPRESSNVVVTERVIQPTSGMIGSLSMHPELANAHNVIVTERVVSGAGVTGISGTTGISGGIGSSGLVGTSMGAGSGALSGAGISGGGIGLSSLGGTASIGHMRSSSDHHFNQTIGSASPSTARSRITKYSTVQYSK

SEQ ID NO: 7 - protein, 408 aa, Homo sapiens
MQDLGGGERMTGFELTEGVKTSGMPEICQEYSGTLRRNSMRECREGGLNMNFMESYFCQKAYAYADEDEGRPSNDCLLIYDIEGVGSPAGSVGCCSFIGEDLDDSFLDTLGPKFKKLADISLGKESYPDLDPSWPPQSTEPVCLPQETEPVVSGHPPISPHFGTTTVISESTYPSGPGVLHPKPILDPLGYGNVTVTESYTTSDTLKPSVHVHDNRPASNVVVTERVVGPISGADLHGMLEMPDLRDGSNVIVTERVIAPSSSLPTSLTIHHPRESSNVVVTERVIQPTSGMIGSLSMHPELANAHNVIVTERVVSGAGVTGISGTTGISGGIGSSGLVGTSMGAGSGALSGAGISGGGIGLSSLGGTASIGHMRSSSDHHFNQTIGSASPSTARSRITKYSTVQYSK

SEQ ID NO: 8 - protein, 438 aa, Homo sapiens
EWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFG

SEQ ID NO: 9 - protein, 439 aa, Mus musculus
EWIKFAAACREGEDNSKRNPIAKIHSDCAANQPVTYRISGVGIDQPPYGIFIINQKTGEINITSIVDREVTPFFIIYCRALNAQGQDLENPLELRVRVMDINDNPPVFSMTTFLGQIEENSNANTLVMKLNATDADEPNNLNSMIAFKIIRQEPSDSPMFIINRKTGEIRTMNNFLDREQYSQYSLVVRGSDRDGGADGMSAESECSITILDVNDNIPYLEQSSYDIEIEENALHSQLVQIRVIDLDEEFSDNWKAIIFFISGNEGNWFEIEMNERTNVGTLKVVKPLDYEAMKNLQLSIGVRNVAEFHQSIISQYRLTATMVTVTVLNVIEGSVFRPGSKTFVVDSRMEANHRVGEFVATDLDTGRASTNVRYEMGNNPENLLVVDSRTGIITLRNRVTMEQYQRLNGEYKGTVLSIDDSLQRTCTGTIVIELSGTGW

SEQ ID NO: 10 - protein, 439 aa, Bos sp.
EWIKFAAACREGEDNSKRNPIAKIHSDCAANQQVTYRISGVGIDQPPYGIFVINQKTGEINITSIVDREVTPFFIIYCRALNSLGQDLEKPLELRVRVLDINDNPPVFSMSTFVGEIEENSNANTLVMVLNATDADEPNNLNSKIAFKIIRQEPSDSPMFIINRYTGEIRTMNNFLDREQYGQYSLAVRGSDRDGGADGMSAECECNIKILDVNDNIPYMELPTQSISIEENSLNSNLLQIRVIDLDEEFSANWMAVIFFISGNEGNWFEIEMNERTNVGTLKVVKPLDFEAMNNLQLSLGVRNKAEFHQSIMSQYKLTATAISVTVLNVVEGSVFRPGSKTFVVNSNMGQNYKIGEYVAWDLDANRPSTTVRYVMGRNPTDLLAIDSKTAIITLRNKVTMEQYKILGGKYQGTILSIDDALQRTCTGTIVINLENGGW

SEQ ID NO: 11 - protein, 563 aa, Homo sapiens
AWITAPVALREGEDLSKKNPIAKIHSDLAEERGLKITYKYTGKGITEPPFGIFVFNKDTGELNVTSILDREETPFFLLTGYALDARGNNVEKPLELRIKVLDINDNEPVFTQDVFVGSVEELSAAHTLVMKINATDADEPNTLNSKISYRIVSLEPAYPPVFYLNKDTGEIYTTSVTLDREEHSSYTLTVEARDGNGEVTDKPVKQAQVQIRILDVNDNIPVVENKVLEGMVEENQVNVEVTRIKVFDADEIGSDNWLANFTFASGNEGGYFHIETDAQTNEGIVTLIKEVDYEEMKNLDFSVIVANKAAFHKSIRSKYKPTPIPIKVKVKNVKEGIHFKSSVISIYVSESMDRSSKGQIIGNFQAFDEDTGLPAHARYVKLEDRDNWISVDSVTSEIKLAKLPDFESRYVQNGTYTVKIVAISEDYPRKTITGTVLINVEDINDNCPTLIEPVQTICHDAEYVNVTAEDLDGHPNSGPFSFSVIDKPPGMAEKWKIARQESTSVLLQQSEKKLGRSEIQFLISDNQGFSCPEKQVLTLTVCECLHGSGCREAQHDSYVGLGP

SEQ ID NO: 12 - protein, 563 aa, Mus musculus
AWITAPVALREGEDLSRKNPIAKIHSDLAEEKGIKITYKYTGKGITEPPFGIFVFDRNTGELNITSILDREETPYFLLTGYALDSRGNNLEKPLELRIKVLDINDNEPVFTQEVFVGSIEELSAAHTLVMKITATDADDPETLNAKVSYRIVSQEPANSHMFYLNKDTGEIYTTSFTLDREEHSSYSLTVEARDGNGQITDKPVQQAQVQIRILDVNDNIPVVENKMYEGTVEENQVNVEVMRIKVTDADEVGSDNWLANFTFASGNEGGYFHIETDTQTNEGIVTLVKEVDYEEMKKLDLSIIVTNKAAFHKSILSKYKATPIPITVKVKNVVEGIHFKSSVVSFRASEAMDRSSLSRSIGNFQVFDEDTGQAAKVTYVKVQDTDNWVSVDSVTSEIKLVKIPDFESRYVQNGTYTAKVVAISKEHPQKTITGTIVITVEDVNDNCPVLVDSVRSVCEDEPYVNVTAEDLDGAQNSAPFSFSIIDQPPGTAQKWKITHQESTSVLLQQSERKRGRSEIPFLISDSQGFSCPERQVLQLTVCECLKGGGCVAAQYDNYVGLGP

SEQ ID NO: 13 - protein, 563 aa, Bos taurus
AWITAPVALREGEDLSRKNPIAKIHSDLAEEKKLKITYKYTGKGITEPPFGVFVFNKDTGELNVTTILDREETPFFLVTGYALDERGKNLEKPIELRIKVLDINDNEPVFTQDVFVGSVEELSAANTLVMKINATDADEPNTLNSKISYRIVSQEPANSPVFYLNKDTGEIYTTSITLDREEYSSYTLIVEARDGNGQITDKPVKQAQVQIRILDVNDNIPVVDRNVYEGSIEENKANVEVLRIKVSDADEVGSDNWLANFTFASGNEGGYFHIETDTQTNEGIVTLIKEVDYETMKNLDLSIIVTNKAAFHKSIKDKYKPTSIPIKVKVKNVKEGIYFKSSTIQFHTSESMEKLSQGQILGKFQVFDEDTGKVPNVKYAKFEDIDNWISVNSATSEIKLVKIPDYESRYVQNGTYTAKIVAITEEYPRKTITGTIVINVEDVNDNCPTLVEPVQTVCDYVPFVNVTAQDLDGPQNSWPFSFSIIDKPAGMAERWKIVRREDNNVLLQQTKRQHGTSEIHFQIADSQGFSCPEEQILRLTVCNCVEGSGCVNMWADSYVGLGP

SEQ ID NO: 14 - protein, 559 aa, Homo sapiens
EWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKDAVCSSSPSVVVSARTLNNRYTGPYTFALEDQPVKLPAVWSITTLNATSALLRAQEQIPPGVYHISLVLTDSQNNRCEMPRSLTLEVCQCDNRGICGTSYPTTSPGTRY

SEQ ID NO: 15 - protein, 559 aa, Mus musculus
EWVKFAKPCREREDNSRRNPIAKITSDFQKNQKITYRISGVGIDQPPFGIFVVDPNNGDINITAIVDREETPSFLITCRALNALGQDVERPLILTVKILDVNDNPPIFSQTIFKGEIEENSASNSLVMILNATDADEPNHMNSKIAFKIVSQEPAGMSMFLISRNTGEVRTLTSSLDREQISSYHLVVSGADNDGTGLSTQCECSIKIKDVNDNFPVLRESQYSARIEENTLNAELLRFQVTDWDEEYTDNWLAVYFFTSGNEGNWFEIETDPRTNEGILKVVKALDYEQVQSMQFSIAVRNKAEFHQSVISQYRVQSTPVTIQVIDVREGISFRPPSKTFTVQRGVSTNKLVGYILGTYQATDEDTGKAASSVRYVLGRNDGGLLVIDSKTAQIKFVKNIDRDSTFIVNKTISAEVLAIDENTGKTSTGTIYVEVPSFNENCPSVVLEKKDICTSSPSVTLSVRTLDRGKYTGPYTVSLEEQPLKLPVMWTITTLNATSALLQAQQQVSPGVYNVPVIVKDNQDGLCDTPESLTLTVCQCDDRSMCRAPIPSREPNTY

SEQ ID NO: 16 - protein, 560 aa, Bos taurus
EWVKFARPCREGEDNSNRNPIAKITSDFEATQKITYHISGIGIDQPPFGIFVVDRNTGEINITAIVDREVTPSFQITCRALNALGQDVEKPLILTVKILDINDNAPVFSQSIFQGEIEENSASNSLVMILNATDADEPNHLNSKIAFKIISQEPAGTSMFLLSRHTGEVRTLTNSLDREQVSSYRLVVSGADRDGEGLSSQCECSIKVKDVNDNFPTFRESQYLARIEENTLSSELLRIQVIDLDEEFTDNWLADFFFTSGNEGHWFEIETDPRTNEGILKVVKSLDYEQVQSMQLGIAVKNKAEFHQSVISQYQVQSTPLIVQVINVREGIAFRPSSKTFTVRKGISSRQLINYVLGTYQAIDEDTGEVASFVRYVLGRNDGGLLMIDSETAGIKFVKNIDRDVTFIVNKTITAEVLAIDGNTGKTSTGTIFVLVPDFNEKCPTIALEKEAVCSSSPSVVVRASPLERDKYAGPYEFSVEHQSLKLPVVWSISGRDATSALLTAPQPVSPGVYRISVMVTDSEGRRCEEPESLTLEVCQCDYRDICPNVYPTSIPNPII

SEQ ID NO: 17 - protein, 559 aa, Homo sapiens
EWIKFAAACREGEDNSKRNPIAKIRSDCESNQKITYRISGVGIDRPPYGVFTINPRTGEINITSVVDREITPLFLIYCRALNSRGEDLERPLELRVKVMDINDNAPVFSQSVYTASIEENSDANTLVVKLCATDADEENHLNSKIAYKIVSQEPSGAPMFILNRYTGEVCTMSSFLDREQHSMYNLVVRGSDRDGAADGLSSECDCRIKVLDVNDNFPTLEKTSYSASIEENCLSSELIRLQAIDLDEEGTDNWLAQYLILSGNDGNWFDIQTDPQTNEGILKVVKMLDYEQAPNIQLSIGVKNQADFHYSVASQFQMHPTPVRIQVVDVREGPAFHPSTMAFSVREGIKGSSLLNYVLGTYTAIDLDTGNPATDVRYIIGHDAGSWLKIDSRTGEIQFSREFDKKSKYIINGIYTAEILAIDDGSGKTATGTICIEVPDINDYCPNIFPERRTICIDSPSVLISVNEHSYGSPFTFCVVDEPPGIADMWDVRSTNATSAILTAKQVLSPGFYEIPILVKDSYNRACELAQMVQLYACDCDDNHMCLDSGAAGIYTEDI

SEQ ID NO: 18 - protein, 559 aa, Mus musculus
EWIKFAAACREGEDNSKRNPIARIRSDCEVSQRITYRISGAGIDRPPYGVFTINPRTGEINITSVVDREITPLFLIHCRALNSRGEDLERPLELRVKVMDVNDNPPVFTQNVYTANIEENSDANALVVKLSATDADEDNHLNSKIAYKIISQEPAGAPMFMVNRYTGEVRTMSNFLDREQHSMYNLLVRGSDRDGATDGLSSECDCRIKILDVNDNFPILEKTSYSASIEENCLSSELIRLQAIDLDEEGTDNWLAQYSILSGNDGNWFEIQTDPKTNEGILKVVKMLDYEQEPNIYLSIGVRNQAEFHHSVASQFQMHSTPVRIQVVNVREGPTFSPSSMTFSLRGGMRGASLMNYVLGTYTAIDMDTGNPATNVRYVIGHDAGSWLKVDSRTGEIQFSREFDMKSKYITDGIYAAQILAIDDGSGRTATGTICIEIPDANDYCPVIYAESRSVCTHASSVRIYVNDHSFGSPFTFCVVDESPDIANIWDIRSINGTSAILMTEQTLSPGPYQIPILVKDSHNRACELPQTVLLDACFCDDHHVCLHSSTTGIYTGDI

SEQ ID NO: 19 - protein, 562 aa, Bos taurus
EWIKFAAACREGEDNSKRNPIARIRSDCEVNQKITYRISGAGIDRPPYGVFTINPRTGEINITSVVDREITPLFLIYCRALNSRGEDLERPLELRVKVMDINDNPPVFTQNVYTASIEENSDANTLVVKLSATDADEDNHLNSKIAYKIVSQEPAGSPMFIMNRYTGEVCTTSNLLDREQHSMYNLLVRGSDRDGATDGLSSECDCRIKVLDVNDNFPTLEKTSYSASIEENCLSSELIRFQAIDLDEEGTDNWLAKYLILSGNDGNWFDIQTDPQTNEGILKVIKTLDYEQVPNIHLSIGVKNQAEFHHSVASQFRMHSTPVRIQVVNVREGPAFQPNSMTFSVREGLKGNSLLNFVLGTYTAIDLDTGNPATNVRYIIGHDAGNWLKIDSRTGEIQFSREFDKKSKYITNGIYTAEILAIDDGSLKTATGTICIEVPDVNDYCPVIVAERETICIASPSILISAAGVNSHPYGSPFTFCVVNEPPGTADIWDITSVNATSAILKAEQTLYSAYYEIPILVKDSFNRACELPQIIELRACDCDINHVCLYSGITDVSTGDG

SEQ ID NO: 20 - protein, 557 aa, Homo sapiens
RWAPIPASLMENSLGPFPQHVQQIQSDAAQNYTIFYSISGPGVDKEPFNLFYIEKDTGDIFCTRSIDREKYEQFALYGYATTADGYAPEYPLPLIIKIEDDNDNAPYFEHRVTIFTVPENCRSGTSVGKVTATDLDEPDTLHTRLKYKILQQIPDHPKHFSIHPDTGVITTTTPFLDREKCDTYQLIMEVRDMGGQPFGLFNTGTITISLEDENDNPPSFTETSYVTEVEENRIDVEILRMKVQDQDLPNTPHSKAVYKILQGNENGNFIISTDPNTNEGVLCVVKPLNYEVNRQVILQVGVINEAQFSKAASSQTPTMCTTTVTVKIIDSDEGPECHPPVKVIQSQDGFPAGQELLGYKALDPEISSGEGLRYQKLGDEDNWFEINQHTGDLRTLKVLDRESKFVKNNQYNISVVAVDAVGRSCTGTLVVHLDDYNDHAPQIDKEVTICQNNEDFAVLKPVDPDGPENGPPFQFFLDNSASKNWNIEEKDGKTAILRQRQNLDYNYYSVPIQIKDRHGLVATHMLTVRVCDCSTPSECRMKDKSTRDVRPNVILGR

SEQ ID NO: 21 - protein, 557 aa, Mus musculus
RWAPIPCSLMENSLGPFPQHIQQIQSDAAQNYTIFYSISGPGVDKEPYNLFYIEKDTGDIYCTRSIDREQYDQFLVYGYATTADGYAPDYPLPLLFKVEDDNDNAPYFETKLTVFSVPENCRSGTSVGQVTAIDKDEPGTLHTRLKYKILQQIPDQPKHFSIHPDTGVITTTTPLLDREKCDTYKLVMEVRDMGGQPFGLFTTGTITISLEDENDNSPYFTQTSYTTEVEENRIDVEILRMVVHDQDLPNTPHSKAVYTILKGNENGNFKITTDPNTNEGVLCVVKPLNYEVSRQVTLQIGVLNEAQFTNAANAQPPTMCTTTVTVKIKDRDEGPECQPPVKVIQSKDGLPAGQELLGYKAVDPETSSGEGLRYEMVGDEDNWFEINKITGDLRTVKVLDRESKFVKNNQYNISVVATDTAGRSCTGTLVVLLEDFNDHPPQIDKEVTICQQEKDFAVLEPIDLDGPDNGPPFQFLLDNSSSKLWTLESQDGKRAILRQRHNLNYNYYSVPIQIQDRHGFSAKHVLSVRVCDCTTPTECRMAVKEERDAKPNIILGK

SEQ ID NO: 22 - protein, 560 aa, Bos taurus
RWAPIPCSLMENSLGPFPQHVQQVQSDAAQNYTIFYSISGPGVDKEPFNLFFIEKDTGDIFCTRSIDREQYQEFPIYAYATTADGYAPEYPLPLVFKVEDDNDNAPYFENKLTVFTVPENCRTGTSVGKVTAIDLDEPDTLHTRLKYKILQQIPNNPRHFTVHPDTGVITTTTPLLDREKCDTYKLIMEVRDMGGQPFGLFNTGTITISLEDENDNAPYFTETSYTVEVEENRIDVEILRMAVHDHDLPNTPHSRAVYQILQGNENGTFKISTDPNTNEAVLCVVKPLNYEVNRQVVLQIGVLNEAQFAKAVNSKTTTTMCTTVVTVKVKDHDEGPECQPPVKVIQSEDCLPTGTELLGYKAVDPERGTGEGLRYKKIQDEDNWFEINEYTGDLKTVKVLDRESTFVKNNQYNVSVIAFDADGRSCTGTLVVFLEDKNDHPPQIKQEELTICRHDKDYVVLEPTDQDGPDNGPPFQFILDNSASKLWTVETRDGKTAILRGRQDLDYDYYTVPIQIKDRHGASATHILPVRVCDCTIPSECRMPSKLSREAALANVFLGK

SEQ ID NO: 23 - protein, 559 aa, Homo sapiens
RWAPIPCSMLENSLGPFPLFLQQVQSDTAQNYTIYYSIRGPGVDQEPRNLFYVERDTGNLYCTRPVDREQYESFEIIAFATTPDGYTPELPLPLIIKIEDENDNYPIFTEETYTFTIFENCRVGTTVGQVCATDKDEPDTMHTRLKYSIIGQVPPSPTLFSMHPTTGVITTTSSQLDRELIDKYQLKIKVQDMDGQYFGLQTTSTCIINIDDVNDHLPTFTRTSYVTSVEENTVDVEILRVTVEDKDLVNTANWRANYTILKGNENGNFKIVTDAKTNEGVLCVVKPLNYEEKQQMILQIGVVNEAPFSREASPRSAMSTATVTVNVEDQDEGPECNPPIQTVRMKENAEVGTTSNGYKAYDPETRSSSGIRYKKLTDPTGWVTIDENTGSIKVFRSLDREAETIKNGIYNITVLASDQGGRTCTGTLGIILQDVNDNSPFIPKKTVIICKPTMSSAEIVAVDPDEPIHGPPFDFSLESSTSEVQRMWRLKAINDTAARLSYQNDPPFGSYVVPITVRDRLGMSSVTSLDVTLCDCITENDCTHRVDPRIGGGGVQLGK

SEQ ID NO: 24 - protein, 559 aa, Mus musculus
RWAPIPCSMLENSLGPFPLFLQQIQSDTAQNYTIYYSIRGPGVDKEPLNLFYVERDTGNLYCTGRVDREQYESFELTAFATTPDGYTPEYPLPLLIKIEDENDNYPIFTQKLYSFTVQENSRIGSIVGEVCATDLDEPDTMHTRLRYSILEQSPSPPMLFTMHPSTGVITTTSAQLDRELIDKYQLLIKVQDMDGQYFGLHTTAKCIITIEDVNDNLPTFTRTTYVTSVEENTVNVEILRLTVQDKDLVNSPNWRANYTILKGNENGNFKIVTDPKTNEGILCVIKPLDYEERQQVTLQIGVVNEAPYTREASSKSPMSTATVTVTVTNQDEGPECIPPMQTVRIQENVPVGTRNDGYKAYDPETRSSSGIRYRKLSDPRGWVTVNEDSGSITIFRALDREAETVRNGIYNITVLALDADGRSCTGTLGIILEDVNDNGPFIPKQTVVICKATMSSAEIVAVDLDDPVNGPPFDFSLESSDSEVQRMWRLTRINDTAARLSYQNDPSFGSYAVPIRVTDRLGLSSVTTLNVLVCDCITESDCTLRSGERTGYADVRLGP

SEQ ID NO: 25 - protein, 555 aa, Bos taurus
RWAPIPCSVPENSLGPFPLFLQQIQSDTAQNYTIYYSISGPGVDKEPRNLFYVERDTGNLFCTASIDRETYPLFELVAFATTPDGYTPEYPLTLVIRIEDENDNAPIFTETSYSFEVFENSKVGTTVGQVCATDQDEPDTLHTRLKYSIIEQFPALPTLFSMHPTTGVITTSSSKLDRELIDKYQLKIKVQDMDGQYFGLQTTAICIINIEDVNDNLPTFTRSSYVASVEENRIDVEILRVAVRDKDLINTANWRANYTILKGNEDGNFKIVTDSQTNEGVLCVVKPLNYEEKQQVTLEIGVVNEAPYTGTSRSTTNMATVTVNVQNQDEGPECDPRVQTVRIKENVPVGTKTIGYKAYDPETGSSSGIRYKKSSDPEGWVDVDKNSGVITILKRLDREARSGVYNISIIASDKDGRTCNGVLGIVLEDVNDNGPVIPQRTVVICKTVMSSAEIVAVDPDEPIHGPPFDFSLEGVSDSEVLRMWRLTKVNDTAARLSYLNDLRFGKYTVPVRVTDRLGQSLVTQLVVILCDCVTPNDCSFRPVSRTGNREVILGK

SEQ ID NO: 26 - protein, 555 aa, Homo sapiens
RWAPIPCSMQENSLGPFPLFLQQVESDAAQNYTVFYSISGRGVDKEPLNLFYIERDTGNLFCTRPVDREEYDVFDLIAYASTADGYSADLPLPLPIRVEDENDNHPVFTEAIYNFEVLESSRPGTTVGVVCATDRDEPDTMHTRLKYSILQQTPRSPGLFSVHPSTGVITTVSHYLDREVVDKYSLIMKVQDMDGQFFGLIGTSTCIITVTDSNDNAPTFRQNAYEAFVEENAFNVEILRIPIEDKDLINTANWRVNFTILKGNENGHFKISTDKETNEGVLSVVKPLNYEENRQVNLEIGVNNEAPFARDIPRVTALNRALVTVHVRDLDEGPECTPAAQYVRIKENLAVGSKINGYKAYDPENRNGNGLRYKKLHDPKGWITIDEISGSIITSKILDREVETPKNELYNITVLAIDKDDRSCTGTLAVNIEDVNDNPPEILQEYVVICKPKMGYTDILAVDPDEPVHGAPFYFSLPNTSPEISRLWSLTKVNDTAARLSYQKNAGFQEYTIPITVKDRAGQAATKLLRVNLCECTHPTQCRATSRSTGVILGK

SEQ ID NO: 27 - protein, 556 aa, Mus musculus
RWAPIPCSMQENSLGPFPLFLQQVQSDAAQNYTVFYSISGRGADQEPLNWFFIERDTGNLYCTRPVDREEYDVFDLIAYASTADGYSADLPLPLPIKIEDENDNYPLFTEAIYAFEVPEGSRLGTVVGTVCATDKDEPDTMHTRLKYSILEQTPPSPGLFSVHPDTGVITTVSHYMDREVVDKYKLIMKVQDMNGQFFGLISTSTCIITVQDSNDNAPTFRQNTYETAVEENTYNVEILRIPVDDKDMINTANWKANFTILKGNENGWFKITTDPVTNEGVLCVVKPLDYEENRQVTLETGVNNEAPFIKDVANRIPTMNRAMVTVHVKDQNEGPECKPPEQYVRIKENSAVGSKINGYKAYDPETKNSNGLRYKKLQDPKDWVSIEEVSGLLTISKTLDREIIAPRNDMYNITVMAIDQEGKSCTGTLAVNIEDVNDNAPEIIQDYIVICKPKMGYTDISAVDPDEPIHGPPFQFNLANTSPEVNRIWTINQVNDTAARLSYQKTADVQIYNVPVTVKDRAGQSATKILRVNLCDCTHPSQCPLRSRSAGITLGK

SEQ ID NO: 28 - protein, 556 aa, Bos taurus
RWAPIPCSMQENSLGPFPLFLQQVQSDAAQNYTIFYSISGRGVDKEPLNLFFIERDTGNLYCTQPVDREEYDVFDLIAYASTADGYSADFPLPLPIRVEDENDNHPIFTEAVYNFEVPESSRVGTTVGVVCATDRDEPDTMHTRLKYSILEQTPRSPGLFSVHPSTGVITTVSHYLDREVADKYSLIMKVQDMDGQFFGLMSTATCIITVKDSNDNLPTFRQNAYEASVEENTVNVEILRIPVEDKDLINTANWRANFTILKGNENGHFKITTDKATNEGVLSVVKPLDYEESHQVVLEIGVANEAPFTRDVALRMTTMNRAVVTVHVKDQDEGPECSPEVQYIRIKENSAVGSKISGYKAYDPETKSSSGLRYKILHDPKEWITVNEGSGSLETYKTLDREVITPKNDLYNITVLAIDQDGRSCTGTLAVSIEDVNDNPPEILQDYLVICKGNMDYVDISAIDHDSSINGAPFYFSLANTSPEINRLWTITRVNDTAARLAYQKNAQFQEYFIPVAVKDRAGLSATKTLRVNLCDCTNPVQCRAARRSADVILGK

SEQ ID NO: 29 - protein, 816 aa, synthetic construct
EWVKFAKPCREREDNSRRNPIAKITSDFQKNQKITYRISGVGIDQPPFGIFVVDPNNGDINITAIVDREETPSFLITCRALNALGQDVERPLILTVKILDVNDNPPIFSQTIFKGEIEENSASNSLVMILNATDADEPNHMNSKIAFKIVSQEPAGMSMFLISRNTGEVRTLTSSLDREQISSYHLVVSGADNDGTGLSTQCECSIKIKDVNDNFPVLRESQYSARIEENTLNAELLRFQVTDWDEEYTDNWLAVYFFTSGNEGNWFEIETDPRTNEGILKVVKALDYEQVQSMQFSIAVRNKAEFHQSVISQYRVQSTPVTIQVIDVREGISFRPPSKTFTVQRGVSTNKLVGYILGTYQATDEDTGKAASSVRYVLGRNDGGLLVIDSKTAQIKFVKNIDRDSTFIVNKTISAEVLAIDENTGKTSTGTIYVEVPSFNENCPSVVLEKKDICTSSPSVTLSVRTLDRGKYTGPYTVSLEEQPLKLPVMWTITTLNATSALLQAQQQVSPGVYNVPVIVKDNQDGLCDTPESLTLTVCQCDDRSMCRAPIPSREPNTYEFGSEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSDYKDDDDKLPETGHHHHHH

SEQ ID NO: 30 - protein, 816 aa, synthetic construct
EWVKFAKPCREREDNSRRNPIAKITSDFQKNQKITYRISGVGIDQPPFGIFVVDPNNGDINITAIVDREETPSFLITCRALNALGQDVERPLILTVKILDVNDNPPIFSQTIFKGEIEENSASNSLVMILNATDADEPNHMNSKIAFKIVSQEPAGMSMFLISRNTGEVRTLTSSLDREQISSYHLVVSGADNDGTGLSTQCECSIKIKDVNDNFPVLRESQYSARIEENTLNAELLRFQVTDWDEEYTDNWLAVYFFTSGNEGNWFEIETDPRTNEGILKVVKALDYEQVQSMQFSIAVRNKAEFHQSVISQYRVQSTPVTIQVIDVREGISFRPPSKTFTVQRGVSTNKLVGYILGTYQATDEDTGKAASSVRYVLGRNDGGLLVIDSKTAQIKFVKNIDRDSTFIVNKTISAEVLAIDENTGKTSTGTIYVEVPSFNENCPSVVLEKKDICTSSPSVTLSVRTLDRGKYTGPYTVSLEEQPLKLPVMWTITTLNATSALLQAQQQVSPGVYNVPVIVKDNQDGLCDTPESLTLTVCQCDDRSMCRAPIPSREPNTYEFGSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGSDYKDDDDKLPETGHHHHHH

SEQ ID NO: 31 - protein, 704 aa, synthetic construct
EWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDGSEPKSCDKTHTCPPCSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSEFDYKDDDDKGSLPETGGHHHHHH

SEQ ID NO: 32 - protein, 701 aa, synthetic construct
EWIKFAAACREGEDNSKRNPIAKIHSDCAANQPVTYRISGVGIDQPPYGIFIINQKTGEINITSIVDREVTPFFIIYCRALNAQGQDLENPLELRVRVMDINDNPPVFSMTTFLGQIEENSNANTLVMKLNATDADEPNNLNSMIAFKIIRQEPSDSPMFIINRKTGEIRTMNNFLDREQYSQYSLVVRGSDRDGGADGMSAESECSITILDVNDNIPYLEQSSYDIEIEENALHSQLVQIRVIDLDEEFSDNWKAIIFFISGNEGNWFEIEMNERTNVGTLKVVKPLDYEAMKNLQLSIGVRNVAEFHQSIISQYRLTATMVTVTVLNVIEGSVFRPGSKTFVVDSRMEANHRVGEFVATDLDTGRASTNVRYEMGNNPENLLVVDSRTGIITLRNRVTMEQYQRLNGEYKGTVLSIDDSLQRTCTGTIVIELSGTGWVPGSGSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKEFDYKDDDDKGSLPETGGHHHHHH

SEQ ID NO: 33 - protein, 5 aa, unidentified
  source 1..5: note = sortase recognition sequence
LPETG

SEQ ID NO: 34 - protein, 6 aa, synthetic construct
HHHHHH

SEQ ID NO: 35 - protein, 5 aa, unidentified
  source 1..5: note = sortase recognition signal sequence
  VARIANT 3: note = X can be any amino acid
LPXTG

SEQ ID NO: 36 - protein, 20 aa, synthetic construct
GGGGSGGGGSGGGGSGGGGS

SEQ ID NO: 37 - protein, 15 aa, synthetic construct
GGGGSGGGGSGGGGS

SEQ ID NO: 38 - protein, 18 aa, synthetic construct
KESGSVSSEQLAQFRSLD

SEQ ID NO: 39 - protein, 14 aa, synthetic construct
EGKSSGSGSESKST

SEQ ID NO: 40 - protein, 8 aa, synthetic construct
GGGGGGGG

SEQ ID NO: 41 - protein, 12 aa, synthetic construct
GSAGSAAGSGEF

SEQ ID NO: 42 - protein, 7 aa, synthetic construct
LPETGGG

SEQ ID NO: 43 - protein, 4 aa, synthetic construct
LPET

SEQ ID NO: 44 - protein, 8 aa, synthetic construct
DYKDDDDK

SEQ ID NO: 45 - protein, 14 aa, synthetic construct
MHHHHHHLPETGGG

SEQ ID NO: 46 - protein, 5 aa, synthetic construct
GGGGS

SEQ ID NO: 47 - protein, 11 aa, synthetic construct
LPETGHHHHHH

(19) United States
(12) Patent Application Publication
(10) Pub. No.: US 2025/0129136 A1
(43) Pub. Date: Apr. 24, 2025

(54) COMPOSITIONS AND METHODS FOR TREATING DISEASE

(71) Applicant: Dana-Farber Cancer Institute, Inc., Boston, MA (US)

(72) Inventors: Mohammad RASHIDIAN, Cambridge, MA (US); Soheil TAVAKOLPOUR, Boston, MA (US)

(21) Appl. No.: 18/719,811

(22) PCT Filed: Dec. 14, 2022
(86) PCT No.: PCT/US2022/081559

§ 371 Date: Jun. 13, 2024

Related U.S. Application Data
(60) Provisional application No. 63/289,595, filed on Dec. 14, 2021.
(60) Provisional application No. 63/368,119, filed on Jul. 11, 2022.

(51) Int. Cl. C07K 14/705; A61K 38/17; A61P 37/00; C07K 14/73; C12N 15/62

(52) U.S. Cl. CPC C07K 14/70517; A61K 38/1709; A61P 37/00; C07K 14/70514; C12N 15/62; C07K 2317/526; C07K 2319/30; C07K 2319/74

(57) ABSTRACT

Aspects of the invention are drawn to compositions and methods for treating and preventing autoimmune disease.

This application claims priority to U.S. Provisional Application No. 63/289,595, filed on Dec. 14, 2021 and U.S. Provisional Application No. 63/368,119, filed on Jul. 11, 2022, the entire contents of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

Aspects of the invention are drawn to a compositions and methods for treating and preventing disease. In embodiments, the disease is an autoimmune disease.

### SEQUENCE LISTING

The instant application contains a Sequence Listing which has been submitted electronically in ASCII format and is hereby incorporated by reference in its entirety. Said ASCII copy, created on [ ], is named [ ] and is [ ] bytes in size.

### BACKGROUND OF THE INVENTION

A variety of autoimmune diseases exist in animals, including humans. Reagents and methods for treating the autoimmune diseases are of interest.

### SUMMARY OF THE INVENTION

Disclosed are reagents and methods for treating autoimmune diseases in animals, including autoimmune blistering diseases. In some embodiments, the reagents include fusion proteins that have a domain including an autoreactive antigen from a diseased animal and a domain that can bind an effector cell. In some embodiments, the effector-cell binding domain can be an Fc portion of an antibody that can bind to an Fc receptor on an effector cell. In some embodiments, the effector cell binding domain can be an antibody that can bind an effector cell.

Aspects of the invention are drawn towards a fusion protein comprising a desmosomal cadherin or fragment thereof, like a desmoglein (encoded by genes DSG1, 2, 3 or 4) or a desmocollin (encoded by genes DSC1, 2 or 3). In some embodiments, SEQ ID NOs. 5-28 (found in Example 4 and in FIG. 13) include all or parts of these amino acid sequences.

In some embodiments, the fusion protein comprises an extracellular (EC) domain of desmosomal cadherin proteins, like desmoglien 3 (Dsg3) or an EC domain of desmoglein 1 (Dsg1), and an isolated immunoglobulin Fc region or a fragment thereof. In embodiments, the fusion protein comprises any one or more of SEQ ID NOs.: 1-4, 29-32 or fragments thereof. In embodiments, the fusion protein comprises an amino acid sequence that is at least 80% identical to SEQ ID Nos 1-4 or 29-32. In embodiments, the fusion protein targets one or more B cells through its desmosomal cadherin portion. In a further embodiment, the targeted B cell comprises an autoreactive anti-Dsg3 B cell or an autoreactive anti-DSG1 B cell. In embodiments, the fusion protein also targets effector cells (e.g., NK cells, macrophages) through its Fc region. In embodiments, the Fc region of the fusion protein comprises an amino acid sequence at least 80% identical to the Fc regions of SEQ ID NO 1, SEQ ID NO 2, SEQ ID NO 3, SEQ ID NO 4, SEQ ID NO 29, SEQ ID NO 30, SEQ ID NO 31 or SEQ ID NO 32. In embodiments, the Fc region comprises an IgG Fc region. In embodiments the EC domain comprises any one or more of the various sequence IDs provided in FIG. 13. In further embodiments, the EC domain comprises EC1, EC2, and EC3. In embodiments the fusion protein further comprises a therapeutic moiety, an imaging moiety, a capturing moiety, or a combination thereof. In further embodiments, the capturing moiety comprises a hydrophilic protein, a bacterial transpeptidase enzyme, a GST tag, a His-Tag, a polyethylene glycol (PEG), or a combination thereof. In another further embodiment, the imaging moiety comprises a fluorophore, a radioisotope, or a combination thereof. In another further embodiment, the therapeutic moiety comprises a cytokine, a toxin, a radiotherapeutic, a T cell-engaging moiety, a natural killer cell engaging moiety, or a combination thereof.

In some embodiments, the fusion protein can comprise a desmosomal cadherin or fragment thereof and an antibody or fragment that can bind an effector cell. In some embodiments, the effector cell can be a T cell.

In some embodiments, the fusion protein can comprise a desmosomal cadherin or fragment thereof and a radioactive isotope.

Aspects of the invention are drawn towards a pharmaceutical combination comprising a fusion protein described herein and a pharmaceutically acceptable carrier.

Aspects of the invention are drawn towards a method of depleting autoreactive anti-Dsg or anti-Dsc B cells, the method comprising administering to a subject a therapeutically effective amount of a fusion protein described herein, or a pharmaceutical composition described herein. In an embodiment, the effective amount is positively correlated with the level of anti-Dsg or Dsc antibodies circulating with the subject.

Aspects of the invention are drawn towards a method of treating an autoimmune disease, the method comprising administering to a subject a therapeutically effective amount of a fusion protein described herein, or a pharmaceutical composition described herein, wherein the subject is suffering from the autoimmune disease. In an embodiment, the disease is selected from the group consisting of an autoimmune blistering disease, lupus, scleroderma, Goodpasture's disease, Graves' disease, and immune-mediated vasculitis. In embodiments, the autoimmune blistering disease comprises pemphigus vulgaris, bullous pemphigoid, mucous membrane pemphigoid, epidermolysis bullosa acquisita, a linear IgA bullous dermatosis, muscle-specific tyrosine kinase (MuSK) myasthenia gravis (MG), PLA2R Membranous Nephropathy, or Hemophilia A with FVIII Alloantibodies. In embodiments, the amount of fusion protein or pharmaceutical composition administered to a subject elicits an immune response against the autoimmune disease.

Aspects of the invention are drawn towards a nucleic acid encoding a fusion protein described herein.

Aspects of the invention are drawn towards a vector comprising a nucleic acid encoding a fusion protein described herein.

Aspects of the invention are drawn towards a cell comprising a vector comprising a nucleic acid encoding a fusion protein described herein.

### BRIEF DESCRIPTION OF THE FIGURES

Certain illustrations, charts, or flow charts are provided to allow for a better understanding for the present invention. It is to be noted, however, that the drawings illustrate only selected embodiments of the inventions and are therefore not to be considered limiting of scope. Additional and equally effective embodiments and applications of the present invention exist.

FIG. 1 shows images of Pemphigus vulgaris in patients, which can result in painful and severe blistering of the skin and mucosa.

FIG. 2 shows a representation of desmosome architecture between keratinocytes (Panel A). Panel B shows a schematic representation of structure of Dsg3 protein; anti-Dsg3 antibodies result in detachment of keratinocytes (acantholysis). The five domains of the extracellular portion of the protein show different levels of immunogenicity. Percentages shown next to each domain is the percentage of PV patients with antibodies specific for that domain. A patient can have antibodies against multiple domains.

FIG. 3 shows schematic representation of the (panel A) Fc-mediated fusion protein (e.g., Dsg is bound by B cell receptor; NK cells/macrophages are bound by Fc receptor), (panel B) antibody-mediated fusion protein (e.g., Dsg is bound by B cell receptor; T cells are bound by antibody; this approach may be called a “B-cell receptor (BCR)-effector cell engager” approach or, where the effector cell is a T cell, a “BCR-T cell engager approach”), and (panel C) radiotherapy approaches. Each of the approaches is designed for targeted killing of pathogenic B cells.

FIG. 4 shows a schematic sortase reaction. A protein equipped with an LPETG sortase tag can be labeled with a triglycine containing sortase substrate. ‘R’ can be any biomolecule of interest. A His-tag (H6) is used to purify the protein.

FIG. 5 shows images of a passive transfer disease model. Adult B6 mice were injected with AK23 hybridoma cells. Hair loss around Panel A. Mouth Panel B. Eyes Panel C. Back Panel D. Panels H&E of loss of keratinocyte cell adhesion. Active immune model. Immunized Dsg3−/− splenocytes transferred to Rag-2−/− mice. Panel E. Significant size difference 25-35 days post splenocytes transfer in mice that received Dsg3−/− splenocytes (bottom) in contrast to mice that received Dsg3+/− splenocytes (top). Panel F. Crusted erosions around the snout and cheeks of mice that received Dsg3-immunized Dsg3−/− splenocytes. Panel G. Intraepithelial blisters in upper esophagus of mice that received Dsg3-immunized Dsg3−/− splenocytes in contrast to mice that received Dsg3-immunized Dsg3+/− splenocytes (Panel H). From Amagai M, Tsunoda K, Suzuki H, Nishifuji K, Koyasu S, Nishikawa T. Use of autoantigen-knockout mice in developing an active autoimmune disease model for pemphigus. J Clin Invest. 2000 March; 105 (5): 625-631. PMCID: PMC292455.

FIG. 6 shows (Panel A) mDsg3-IgG2a was labeled with Alexa647 by sortase. (Panel B) SDS-PAGE, in-gel fluorescence and western blot characterization of mDsg3 (EC1-3)-IgG2a (expected MW ˜80 kDa). (Panel C) Flow cytometry analysis of anti-Dsg3 F779 and AK23 hybridoma cells with mDsg3-IgG2a-Alexa647, and Nalm6 as a negative control. (Panel D) ELISA analyses confirmed the binding of the mDsg3-EC1-3-IgG2a to patient-derived P3F3 and mouse-derived AK23 anti-Dsg3 Abs. (Panel E) Staining of mouse RAW264.7 macrophage cells with mDsg3-IgG2a-Alexa647; Nalm6 cells was used as a negative control as shown in C: purple histogram).

FIG. 7 shows in vitro Fc-mediated cytotoxicity assay for evaluation of mDsg3-mFc chimeric protein. Anti-Dsg3 AK23 (Panel A) and F779 (Panel B) cells, and irrelevant hybridoma or Nalm6 control cells were co-cultured with mouse RAW264.7 macrophage cells with 10 nM of mDsg3-IgG2a construct. The killing efficacy was measured via standard live-dead assay after the indicated time points. The results were normalized to the 0 nM experiments; ***p<0.001.

FIG. 8 shows schematics of (Panel A) using click reaction to fuse Dsg3 and anti-CD3 scFvs. (Panel B) Using sortase to directly make Dsg3-anti-CD3 fusion protein. (Panel C) SDS-PAGE (Coomassie Staining) characterization of anti-CD19 and anti-CD3 scFv and the fusion protein after direct sortase reaction, (each scFv is ˜35 kDa in size); arrow shows the fusion (expected mass ˜70 kDa). (Panel D) Flow cytometry staining of CD19 expressing Nalm6 and human CD3 expressing Jurkat cells with the anti-CD19-anti-CD3 fusion protein made via the direct sortase approach. Click-chemistry fusion provided similar results. Results are representative (n=3-5 for each).

FIG. 9 shows data of (Panel A) mDsg3-EC1-3 was labeled with Alexa647 using sortase. (Panel B) SDS-PAGE, in-gel fluorescence and western blot characterization of mDsg3 (EC1-3) and the Alexa labeled product (expected MW: ˜40 kDa). (Panel C) Flow cytometry analysis of anti-Dsg3 expressing F779, PVB-28 and AK23 hybridoma cells with mDsg3-Alexa647; Nalm6 cells was used as a negative control. Results are representative (n=3-5 for each).

FIG. 10 shows (Panel A) mDsg3-EC1-3 was site-specifically labeled with DOTA using sortase. It will be radiolabeled using 225-Ac radioisotope as shown. (Panel B) SDS-PAGE (Coomassie Staining) characterization of mDsg3 (EC1-3) (lower band; lane 2) and DOTA labeled mDsg3 (EC1-3) (slightly higher band; lane 3); lane #1 is marker. (Panel C) LC-MS mass spectrophotometry analysis of mDsg3 (EC1-3) (Upper) and DOTA labeled mDsg3 (EC1-3) (Lower) along with expected and observed mass. (Panel D) DOTA labeling does not impact binding affinity. Flow cytometry staining of anti-Dsg3 expressing F779 cells with mDsg3 (EC1-3) (Positive Control) and DOTA labeled mDsg3 (EC1-3) (Lower) using secondary anti-FLAG antibody staining.

FIG. 11 shows Schematic representation of the (Panel A) Fc-mediated, and (Panel B) BCR-T cell engager approaches. Each of the approaches is designed for targeted killing of pathogenic B cells.

FIG. 12 shows (Panel A) Schematic representation of the Fc-mediated approach to target and kill the autoreactive pathogenic B cells. (Panel B) In vitro characterization of the Dsg3-Fc constructs. F779 (Nalm6-leukemia cells engineered with patient-derived anti-Dsg3 antibodies) were incubated with varying concentration of mDsg3-mFc (IgG2a) (top) or hDsg3-hFc (IgG1) (bottom) and analyzed by flow cytometry to calculate the EC50 values. (Panel C) Schematic illustration of the in vivo experimental timeline. (Panel D) Kaplan-Meier survival curve. Log rank survival analysis of treated and control mice showing improved survival of mice receiving treatment. Control vs. treatment (**p<0.005) (Panel E) Upon BSC<2 blood was collected from treatment or control and flow analyzed for presence of anti-Dsg3 expression on the surface of human-CD19⁺ GFP⁺ F779 cells that escaped the treatment. The treatment group showed near-complete antigen-loss. The data is representative of 5 mice.

FIG. 13 shows multiple sequence alignment of desmosomal cadherin ectodomains (SEQ ID NOs. 8-28). Human, mouse and bovine Dsg1-4 (blue) and Dsc1-3 (orange) amino acid sequences were aligned for each EC domain. Residues conserved across both families are highlighted grey; those conserved within Dsgs or Dscs only, are highlighted blue and orange, respectively. Secondary structure corresponding to the human Dsg2 structure and the human Dsc1 structure are displayed above (blue) and below (orange) the alignment. O-linked glycans are shown as violet hexagons; N-linked glycans are shown as wheat hexagons and, where not conserved, individual glycosylated residues are additionally boxed. Disulfide bonds are shown by yellow lines above the alignment for Dsgs and below the alignment for Dscs. Interface residues for which >10% solvent accessible surface area is buried in the strand-swapped homodimers are indicated by blue (Dsg2), orange (Dsc1) and salmon (Dsc2) bars. Dsg1, which lacks an EC5 domain, is excluded from the EC5 alignment (see Harrison et al., Structural basis of desmosomal cadherin adhesion, Proceedings of the National Academy of Sciences June 2016, 113 (26) 7160-7165).

FIG. 14 shows a non-limiting, exemplary clinical presentation of mucosal-dominant pemphigus vulgaris, which results in painful and severe erosions in the mucosa.

FIG. 15 shows schematic representation of the (Panel A) Fc-mediated, and (Panel B) BCR-T cell engager approaches. Each of the approaches is designed for targeted killing of pathogenic B cells.

FIG. 16 shows (Panel A) Site-specific labeling of Dsg3-(EC1-3)-Fc construct via sortase. (Panel B) SDS-PAGE, in-gel fluorescence and western blot characterization of mDsg3-mFc, as indicated on the right side of the gel. (Panel C) Flow cytometric analyses of anti-Dsg3+F779, PVB28, and AK23 hybridoma cells with murine and human Dsg3-Fc-labeled with Alexa Fluor 647; Nalm6 is used as control. (Panels D&E-G&H) EC₅₀ values were determined for the mDsg3-mFc and hDsg3-hFc constructs using the F779 (for the Dsg3 portion), and using the Raw cells, and human PBMCs (for the Fc portions), via flow cytometric analyses. Note that only ˜50% of hPBMCs are Fc-R+ cells, further showing the specificity of the construct. (Panel F) ELISA analyses further confirmed the specificity of the constructs. Results are representative of multiple experiments (at least n=3 for each experiment).

FIG. 17 shows in vitro cytotoxicity assay to assess the efficacy of Dsg3-Fc engineered proteins. The assays started with 200,000 pre-coated effector cells in 48-well plates. ˜4-6 hours after the coating, 20,000 target cells, Dsg3-Fc treatment (at different concentrations as indicated), and 1 mM calcium were added. The live GFP+ cells were counted at 24 and 48 h later via flow cytometric analyses (all target cells are GFP+). At least n=3 were used for each condition. Significant specific killing was observed for all tested conditions compared to control cells (***p<0.001).

FIG. 18 shows the presence of anti-Dsg3 autoantibody does not neutralize the in vitro efficacy of the treatment. Fc-mediated cytotoxicity assay was performed with the identical setting as explained in FIG. 17, but in the presence of AK23 antibody as indicated. No difference in the killing efficacy was detected when the AK23 antibody was added to the mixture. Results are representative of at least n=3 for each experiment.

FIG. 19 shows the hDsg3-anti-CD3e scFv construct specifically stains F779 and Jurkat cells (human T cells), but not the control Nalm-6 cells.

FIG. 20 shows the presence of autoantibodies does not inhibit the efficacy of the treatment. Mice received 3 doses of IVIG (days −3, −2, −1; 16 mg/kg), allowing for a more rapid engraftment of the luciferase⁺ PVB28 cells (1 million cells injected on day 0). mDsg3-EC14-mFc is used as the treatment (three times per week for two weeks, and then once per week, starting from day 4). Control mice received an isotype IgG2a. Auto-Abs group received mix of AK23, AK19 and AK18 (150 μg total), twice per week for two weeks starting a day prior to initiation of treatment. (Panel A) BLI imaging showed a slower signal enhancement in treatment-recipient mice even in the presence of autoantibodies. (Panel B) The survival rate is significantly higher in both treatment-recipient groups compared to the control group (P value=0.0023). Also, the median of survival for autoantibodies+treatment (Auto-Abs+Tx) group was 38 days which is higher than the treatment group (median survival=35 days; P value=0.048)). (Panel C) Flow cytometric analysis at the end point showed complete depletion of PVB28 cells in the spleen and significant loss of anti-Dsg3⁺ on PVB28 cells in the bone marrow of the treatment-recipient mice (data representative of n=5 for each group).

FIG. 21 shows (Panels A-D) Passive transfer model. Adult RAG2−/− mice injected with AK23 hybridoma cells. Hair loss around mouth (Panel A), eyes (Panel B), and back (Panel C). (Panel D) H&E of loss of keratinocyte cell adhesion. (Panels E-H) Active immune model. Immunized Dsg3−/− splenocytes transferred to Rag-2−/− mice. (Panel E) Significant size difference 25-35 days post splenocytes transfer in Rag-2−/− mice that received immunized Dsg3−/− splenocytes (bottom) in contrast to mice that received Dsg3+/− splenocytes (top). (Panel F) Crusted erosions around the snout and cheeks of mice that received Dsg3-immunized Dsg3−/− splenocytes. (Panel G) Intraepithelial blisters in upper esophagus of mice that received Dsg3-immunized Dsg3−/− splenocytes in contrast to mice that received Dsg3-immunized Dsg3+/− splenocytes. (Panel H) Same for skin around the snout.

FIG. 22 shows that treatment does not result in cytokine storm and toxicity in immunocompetent mice, even in the presence of autoantibodies. (Panel A) The schedule for injecting mice with treatment (mDsg3-EC14-mFc) and/or the mix of autoantibodies (AK23, AK19, AK18). (Panels B and C) Treatment-recipient mice, with or without autoantibodies, continued to gain normal weight with no observed toxicities (n=6 for each group; 3 male and 3 female). (Panel D) ELISA analyses showed no cytokine storm in any of the three groups that received either only the treatment, or autoantibodies, or both autoantibodies and the treatment (using ELISA MAX™ Standard Set Mouse (BioLegend)). Sera from 6 mice that did not receive anything were used for healthy control in each gender. Samples from 3 mice that received 2 mg/kg of lipopolysaccharide (LPS) were used to mimic a cytokine storm in each gender. Unpaired t-test was used to test for significant differences between groups. (ns, P>0.05; *, P≤0.05; **, P≤0.01; ***, P≤0.001; ****, P≤0.0001).

FIG. 23 shows that the treatment (mDsg3-EC1-4-mFc) inhibits pathogenic effects of AK23 antibody. All animals received AK23 (12 mg/kg; subcutaneous); the AK23+treatment cohort received mDsg3-mFc 1 h later (15 mg/kg, i.p). Panels A and B show the AK23 group showed significant hair-loss by tape-stripping performed 72 h later (Panel A), and showed severe PV phenotype (i.e. hair-loss and mucosal erosions (not shown)) at day 5 (Panel B); however, the “AK23+Treatment” group was similar to the control healthy mice (Panels A and B); data representative of n=3 for each cohort. (Panel C) AK23 cohort showed weight-loss; however, weight-gain was normal in the mice that received AK23 and the treatment. (Panel D) ELISA analyses of IL6 on sera collected at 72 h showed high levels of the cytokine in the AK23 cohort, but it was similar to healthy control in the “AK23+treatment” group. (Panel E) Survival analyses of the experiment. All AK23 mice developed severe PV and had to be euthanized <7 days. All the treatment mice behaved normal. The experiment was ended at day 60 (n=3 for each cohort).

### DETAILED DESCRIPTION OF THE INVENTION

Disclosed herein are reagents and methods for treating diseases or conditions in which autoreactive B cells produce autoantibodies in affected animals (e.g., humans, dogs, cats, horses). The reagents and methods are designed to target and deplete the autoreactive B cells. In some embodiments, the reagents and methods are for treating autoimmune blistering diseases, like pemphigus vulgaris, bullous pemphigoid, mucous membrane pemphigoid, epidermolysis bullosa acquisita, linear IgA bullous dermatosis, pemphigus foliaceus, intraepidermal neutrophilic IgA dermatosis, paraneoplastic pemphigus and the like. In some embodiments, the reagents can contain an autoreactive B cell epitope that binds to a B cell receptor on an autoreactive B cell. In some embodiments, the autoreactive B cell epitope can be from extracellular domains of desmoglein (Dsg) or desmocollin (Dsc) molecules. In various embodiments, in addition to the autoreactive B cell epitope, the reagents also can contain an effector-cell binding region that can bind to various effector cells, like NK cells, macrophages and T cells. When the autoreactive epitope of the reagent binds to an autoreactive B cell, the effector cells bound to the effector-cell binding region of the reagent are positioned in close proximity to the B cell and can deplete the B cell.

In some embodiments, a reagent having an autoreactive B cell epitope that binds to a B cell receptor on an autoreactive B cell can have an effector-cell binding region that includes an Fc portion of an antibody. The Fc portion of an antibody can bind to cells that have an Fc receptor (FcR), including effector cells like macrophages, natural killer (NK) cells, and the like. These effector cells can deplete the autoreactive B cell. These therapeutics can be called Fc-mediated therapeutics.

In some embodiments, a reagent having an autoreactive B cell epitope that binds to a B cell receptor on an autoreactive B cell can have an effector-cell binding region that includes an antibody that can bind to an effector cell. In some embodiments, the antibody can be specific for T cells (anti-CD3 antibody). The antibody can be specific for cytotoxic T cells. The T cells can deplete the autoreactive B cell. These therapeutics can be called B cell receptor (BCR)-effector cell engagers. Therapeutics that bind to an effector cell that is a T cell can be called BCR-T cell engagers.

In some embodiments, the reagent molecules as described above may have a spacer between or connecting the autoreactive B cell epitope and the effector-cell binding region. In some embodiments, the spacer can be an amino acid spacer. The spacer can be a flexible spacer. The flexible spacer can contain glycine and serine amino acid residues. In some embodiments, the spacer connecting the autoreactive B cell epitope and the effector-cell binding region can be made using “click” chemistry. In some embodiments, the spacer can include a C-to-C bond made using copper-mediated azide-alkyne cycloaddition chemistry. In some embodiments, the spacer can include a N-to-C bond made using a sortase enzyme.

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

The term “subject” or “patient” can refer to any organism to which aspects of the invention can be performed, e.g., for experimental, diagnostic, prophylactic, and/or therapeutic purposes. Subjects to which methods as described herein are performed comprise mammals, such as primates, for example humans. For veterinary applications, a wide variety of subjects are suitable, e.g., livestock such as cattle, sheep, goats, cows, swine, and the like; poultry such as chickens, ducks, geese, turkeys, and the like; and domesticated animals and pets such as dogs and cats. For diagnostic or research applications, a wide variety of mammals are suitable subjects, including rodents (e.g., mice, rats, hamsters), rabbits, primates, and swine such as inbred pigs and the like. The term “living subject” can refer to a subject noted herein or another organism that is alive. The term “living subject” can refer to the entire subject or organism and not just a part excised (e.g., a liver or other organ) from the living subject. The term “normal subject” can refer to a subject that is not afflicted with a disease or condition, such as a subject that is not afflicted with a cancer.

As used herein, the phrase “therapeutic agent” can refer to any agent that elicits a desired pharmacological effect when administered to a subject. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is any substance that can be used to alleviate, ameliorate, relieve, inhibit, delay onset of, reduce severity of, and/or reduce incidence of one or more symptoms or features of a disease, disorder, and/or condition.

The term “therapeutically effective amount”, as used herein, can refer to an amount of a therapeutic agent whose administration, when viewed in a relevant population, correlates with or is reasonably expected to correlate with achievement of a particular therapeutic effect. The therapeutic effect may be objective (i.e., measurable by some test or marker) or subjective (i.e., subject gives an indication of or feels an effect). In some embodiments, a therapeutically effective amount of a substance is an amount that is sufficient, when administered to a subject suffering from or susceptible to a disease, disorder, and/or condition, to treat, diagnose, prevent, and/or delay and/or alleviate one or more symptoms of the disease, disorder, and/or condition. Disease progression can be monitored by clinical observations, laboratory and imaging investigations apparent to a person skilled in the art. A therapeutically effective amount is administered in a dosing regimen that can comprise multiple unit doses. For a therapeutic agent, a therapeutically effective amount (and/or an appropriate unit dose within an effective dosing regimen) can vary, for example, depending on route of administration, on combination with other pharmaceutical agents. Also, the specific therapeutically effective amount (and/or unit dose) for a patient can depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific fusion protein employed; the duration of the treatment; and like factors as is well known in the medical arts. Furthermore, an effective amount may be administered via a single dose or via multiple doses within a treatment regimen. In some embodiments, individual doses or compositions are considered to contain a “therapeutically effective amount” when they contain an amount effective as a dose in the context of a treatment regimen. Those of ordinary skill in the art will appreciate that a dose or amount may be considered to be effective if it is or has been demonstrated to show statistically significant effectiveness when administered to a population of patients; a particular result need not be achieved in a particular individual patient in order for an amount to be considered to be therapeutically effective as described herein.

The word “treating” can refer to the medical management of a subject, e.g., an animal, including human, with the intent that a prevention, cure, stabilization, or amelioration of the symptoms or condition will result. This term includes active treatment, that is, treatment directed specifically toward improvement of the disorder; palliative treatment, that is, treatment designed for the relief of symptoms rather than the curing of the disorder; preventive treatment, that is, treatment directed to prevention of disorder; and supportive treatment, that is, treatment employed to supplement another specific therapy directed toward the improvement of the disorder. The term “treatment” also includes symptomatic treatment, that is, treatment directed toward constitutional symptoms of the disorder. The terms “treat” or “treatment” can also refer to both therapeutic treatment and prophylactic or preventative measures, wherein the object is to prevent or slow down (lessen) an undesired physiological change or disorder. “Treatment” can also refer to prolonging survival as compared to expected survival if not receiving treatment. Those in need of treatment include those already with the condition or disorder as well as those prone to have the condition or disorder or those in which the condition or disorder is to be prevented.

“Treating” a condition with the compounds of the invention involves administering such a compound, alone or in combination and by any appropriate means, to a patient. Treatment can be administered to a subject who does not exhibit signs of a disease, disorder, and/or condition (e.g., prior to an identifiable disease, disorder, and/or condition), and/or to a subject who exhibits only early signs of a disease, disorder, and/or condition for the purpose of decreasing the risk of developing pathology associated with the disease, disorder, and/or condition.

The term “administration” can refer to introducing a pharmaceutical composition or formulation as described herein into a subject. One route of administration of the composition is intravenous administration. However, any route of administration, such as oral, topical, subcutaneous, peritoneal, intra-arterial, inhalation, vaginal, rectal, nasal, introduction into the cerebrospinal fluid, or instillation into body compartments can be used.

Herein, “allergen” can refer to a substance that can cause an allergic reaction. For example, an allergen can produce an abnormally strong or vigorous immune response (e.g., hypersensitivity of the immune system). In some embodiments, hypersensitivity can be type-I hypersensitivity. In some embodiments, the hypersensitivity can be mediated by IgE.

Herein, “antibody” can refer to a molecule or molecules that binds an antigen. Herein, “antibody” can refer to all types of antibodies, fragments and/or derivatives. Antibodies include polyclonal and monoclonal antibodies of any suitable isotype or isotype subclass. Herein, antibody can refer to, but not be limited to Fab, F(ab′)2, Fab′ single chain antibody, Fv, single chain, mono-specific antibody, bi-specific antibody, tri-specific antibody, multi-valent antibody, chimeric antibody, canine-human chimeric antibody, chimeric antibody, humanized antibody, human antibody, CDR-grafted antibody, shark antibody, nanobody (e.g., antibody consisting of a single monomeric variable domain), camelid antibody (e.g., from the Camelidae family) microbody, intrabody (e.g., intracellular antibody), and/or de-fucosylated antibody and/or derivative thereof. Mimetics of antibodies are also provided. In some embodiments, the antibodies disclosed herein are active agents that are part of the compounds disclosed herein that can cross the blood brain barrier.

“Antibody” or “antigen-binding polypeptide” can refer to a polypeptide or a polypeptide complex that specifically recognizes and binds to an antigen. An antibody can be a whole antibody and any antigen binding fragment or a single chain thereof. For example, “antibody” can include any protein or peptide containing molecule that comprises at least a portion of an immunoglobulin molecule having biological activity of binding to the antigen. Non-limiting examples of a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. As used herein, the term “antibody” can refer to an immunoglobulin molecule and immunologically active portions of an immunoglobulin (Ig) molecule, i.e., a molecule that contains an antigen binding site that specifically binds (immunoreacts with) an antigen. By “specifically binds” or “immunoreacts with” is meant that the antibody reacts with one or more antigenic determinants of the desired antigen and does not react with other polypeptides.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” can include aptamers (such as spiegelmers), minibodies, and diabodies. The term “antibody fragment” can also include any synthetic or genetically engineered protein that acts like an antibody by binding to a specific antigen to form a complex. Antibodies, antigen-binding polypeptides, variants, or derivatives described herein include, but are not limited to, polyclonal, monoclonal, multispecific, human, humanized or chimeric antibodies, single chain antibodies, epitope-binding fragments, e.g., Fab, Fab′ and F(ab′)₂, Fd, Fvs, single-chain Fvs (scFv), single-chain antibodies, dAb (domain antibody), minibodies, disulfide-linked Fvs (sdFv), fragments comprising either a VL or VH domain, fragments produced by a Fab expression library, and anti-idiotypic (anti-Id) antibodies.

A “single-chain variable fragment” or “scFv” refers to a fusion protein of the variable regions of the heavy (VH) and light chains (VL) of immunoglobulins. A single chain Fv (“scFv”) polypeptide molecule is a covalently linked VH: VL heterodimer, which can be expressed from a gene fusion including VH- and VL-encoding genes linked by a peptide-encoding linker. (See Huston et al. (1988) Proc Nat Acad Sci USA 85 (16): 5879-5883). In some aspects, the regions are connected with a short linker peptide of ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. A number of methods have been described to discern chemical structures for converting the naturally aggregated, but chemically separated, light and heavy polypeptide chains from an antibody V region into an scFv molecule, which will fold into a three-dimensional structure substantially similar to the structure of an antigen-binding site. See, e.g., U.S. Pat. Nos. 5,091,513; 5,892,019; 5,132,405; and 4,946,778, each of which are incorporated by reference in their entireties.

Antibody molecules obtained from humans fall into five classes of immunoglobulins: IgG, IgM, IgA, IgE and IgD, which differ from one another by the nature of the heavy chain present in the molecule. Those skilled in the art will appreciate that heavy chains are classified as gamma, mu, alpha, delta, or epsilon (Y, u, a, 8, ¿) with some subclasses among them (e.g., γ1-γ4). Certain classes have subclasses as well, such as IgG₁, IgG₂, IgG₃ and IgG₄ and others. The immunoglobulin subclasses (isotypes) e.g., IgG₁, IgG₂, IgG₃, IgG₄, IgG₅, etc. are well characterized and are known to confer functional specialization. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region. Immunoglobulin or antibody molecules described herein can be of any type (e.g., IgG, IgE, IgM, IgD, IgA, and IgY), class (e.g., IgG1, IgG2, IgG3, IgG4, IgA1 and IgA2) or subclass of an immunoglobulin molecule.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells, or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. The variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. The term “antigen-binding site,” or “binding portion” can refer to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” can refer to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” VH and VL regions, which contain the CDRs.

The term “monoclonal antibody” or “mAb” or “Mab” or “monoclonal antibody composition”, as used herein, can refer to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

Herein, “autoantibody” can refer to an antibody produced by an animal that binds to or reacts with an antigen from the animal (e.g., a self-antigen instead of a “foreign” antigen). Generally (e.g., in absence of disease or a condition), animals are tolerant to self-antigens and do not produce autoantibodies.

Herein, “autoimmune” can refer to an immune response (e.g., antibody production) against a self-antigen. “Autoimmune disease” can refer to a disease or condition resulting from the immune response to self-antigens.

Herein, “autoreactive” can refer to components of an autoimmune response. For example, a B cell that produces an antibody reactive against a self-antigen can be referred to as an autoreactive B cell. The antibody can be referred to as an autoreactive antibody. The self-antigen to which the autoantibody is reactive can be called an autoreactive antigen.

Herein, “B cell” can refer to a type of lymphocyte that functions in the humoral immune response of an animal. B cells produce antibodies, that are displayed on the surface of the B cell. Plasma cells, which are a more differentiated form of B cells, can secrete antibodies.

Herein, “B cell receptor” or “BCR” can refer to the antibody produced by a B cell, which is displayed on the surface, together with a signal transduction moiety.

Herein, “depleting” can refer removing or diminishing. Herein, depleting can refer to removing autoreactive B cells. Depleting can refer to killing of autoreactive B cells.

Herein, “desmoglein” and “desmocollin” can refer to families of cadherins that are involved in formation of desmosomes.

Herein, “effector cell” can refer to cells that can deplete autoreactive B cells. In some embodiments, effector cells can be macrophages, NK cells, T cells, and the like.

Herein, “epitope” can refer to the part of an antigen that binds to an antibody (e.g., to a B cell receptor). This epitope can be called a B cell epitope, which can be distinguished from a T cell epitope.

The term “epitope” can include any protein determinant that can specifically bind to an immunoglobulin, a scFv, or a T-cell receptor. The variable region allows the antibody to selectively recognize and specifically bind epitopes on antigens. For example, the VL domain and VH domain, or subset of the complementarity determining regions (CDRs), of an antibody combine to form the variable region that defines a three-dimensional antigen-binding site. This quaternary antibody structure forms the antigen-binding site present at the end of each arm of the Y. Epitopic determinants can consist of chemically active surface groupings of molecules such as amino acids or sugar side chains and usually have specific three-dimensional structural characteristics, as well as specific charge characteristics. For example, antibodies can be raised against N-terminal or C-terminal peptides of a polypeptide. More specifically, the antigen-binding site is defined by three CDRs on each of the VH and VL chains (i.e., CDR-H1, CDR-H2, CDR-H3, CDR-L1, CDR-L2 and CDR-L3).

Herein, “extracellular domain” can refer to the part of a cellular membrane protein that extends from the exterior surface of the cell membrane of a cell.

Herein, “Fc region” (fragment crystallizable region) can refer to the tail region of an antibody that binds to Fc receptors. The Fc region can be recovered when an antibody is digested by papain.

Herein, “Fc receptor” or “FcR” can refer to a receptor that can bind Fc regions of antibodies. Fc receptors are present on the surface of certain cells, including natural killer cells and macrophages.

As used herein, the terms “immunological binding,” and “immunological binding properties” can refer to the non-covalent interactions of the type which occur between an immunoglobulin molecule and an antigen for which the immunoglobulin is specific. In some embodiments, these terms may describe binding of an Fc domain to an Fc receptor. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the equilibrium binding constant, K_{D}. (See, generally, Davies et al. (1990) Annual Rev Biochem 59:439-473). An antibody of the invention can specifically bind to an epitope when the equilibrium binding constant (K_{D}) is ≤1 μM, ≤10 μM, ≤10 nM, ≤10 pM, or ≤100 pM to about 1 pM, as measured by kinetic assays such as radioligand binding assays or similar assays known to those skilled in the art, such as BIAcore or Octet (BLI). For example, in some embodiments, the K_{D} is between about 1E-12 M and a K_{D} about 1E-11 M. In some embodiments, the K_{D} is between about 1E-11 M and a K_{D} about 1E-10 M. In some embodiments, the K_{D} is between about 1E-10 M and a K_{D} about 1E-9 M. In some embodiments, the K_{D} is between about 1E-9 M and a K_{D} about 1E-8 M. In some embodiments, the K_{D} is between about 1E-8 M and a K_{D} about 1E-7 M. In some embodiments, the K_{D} is between about 1E-7 M and a K_{D} about 1E-6 M. For example, in some embodiments, the K_{D} is about 1E-12 M while in other embodiments the K_{D} is about 1E-11 M. In some embodiments, the K_{D} is about 1E-10 M while in other embodiments the K_{D} is about 1E-9 M. In some embodiments, the K_{D} is about 1E-8 M while in other embodiments the K_{D} is about 1E-7 M. In some embodiments, the K_{D} is about 1E-6 M while in other embodiments the K_{D} is about 1E-5 M. In some embodiments, for example, the K_{D} is about 3 E-11 M, while in other embodiments the K_{D} is about 3E-12 M. In some embodiments, the K_{D} is about 6E-11 M. “Specifically binds” or “has specificity to,” can refer to an antibody that binds to an epitope via its antigen-binding domain, and that the binding entails some complementarity between the antigen-binding domain and the epitope. For example, an antibody is said to “specifically bind” to an epitope when it binds to that epitope, via its antigen-binding domain more readily than it would bind to a random, unrelated epitope.

Herein, “fusion protein” can refer to proteins created by joining genes that originally were separate genes.

Herein, “macrophage” can refer to a type of phagocytic white blood cell.

Herein, “natural killer cell” or “NK cell” can refer to a type of cytotoxic lymphocyte.

Herein, “polypeptide” as used herein can encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids, can refer to “polypeptide” herein, and the term “polypeptide” can be used instead of, or interchangeably with any of these terms. “Polypeptide” can also refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology but is not necessarily translated from a designated nucleic acid sequence. It may be generated in any manner, including by chemical synthesis. “Recombinant” as it pertains to polypeptides (such as antibodies) or polynucleotides refers to a form of the polypeptide or polynucleotide that does not exist naturally, a non-limiting example of which can be created by combining polynucleotides or polypeptides that would not normally occur together.

Herein, “sortase” can refer to enzymes from bacteria that recognize and cleave a carboxy-terminal recognition signal, generally in a peptide, polypeptide or protein. In some embodiments, the recognition signal can include the amino acid motif Leu-Pro-X-Thr-Gly, where “X” can be any amino acid.

Herein, “T cell” can refer to types of lymphocytes that functions in the cellular immune response of an animal.

Herein, “targets” when used as a verb, can refer to a cell or molecule that specifically attacks something else (e.g., a fusion protein attaches to a B cell and brings along an effector cell that depletes the B cell; the fusion protein and the effector cell target the B cell). “Target” when used as a noun, can refer for example to the B cell that is depleted.

### Autoimmune Disease

The diseases or conditions that the reagents and methods disclosed here are designed to treat can be autoimmune conditions or diseases. The diseases or conditions treated can involve those in which autoreactive B cells produce autoreactive antibodies specific for self or autoreactive antigens. The diseases can include, for example, autoimmune blistering disease, lupus, scleroderma, Goodpasture's disease, Graves' disease, immune-mediated vasculitis, and the like.

The diseases can include (MuSK) myasthenia gravis (MG), which is a rare, frequently more severe subtype of MG with different pathogenesis and unusual clinical features.

The diseases can include PLA2R membranous nephropathy. PLA2R is an autoantigen present in glomerular podocytes. Membranous nephropathy (MN) can occur when circulating antibodies permeate the glomerular basement membrane and in the subepithelial space, form immune complexes with epitopes on podocyte membranes.

The diseases can include hemophilia A with FVIII alloantibodies.

In some embodiments, the diseases or conditions can include autoimmune blistering diseases, like pemphigus vulgaris, bullous pemphigoid, mucous membrane pemphigoid, epidermolysis bullosa acquisita, linear IgA bullous dermatosis, pemphigus foliaceus, intraepidermal neutrophilic IgA dermatosis, and paraneoplastic pemphigus.

In some embodiments, the diseases or conditions can be present in animals, including humans, dogs, cats or horses.

In some embodiments, the autoreactive antibodies present in animals with the diseases or conditions can be specific for autoreactive antigens, that include molecules like desmoplakin, envoplakin, periplakin, plectin, bullous pemphigoid antigen 1, corneodesmosin, microtubule actin crossing-linking factor, epiplakin and cadherin.

In some embodiments, the autoreactive antigens can be desmosomal cadherins like desmoglein-1 (Dsg1), desmoglein-2 (Dsg2), desmoglein-3 (Dsg3), desmoglein-4 (Dsg4), desmocollin-1 (Dsc1) desmocollin-2 (Dsc2), and desmocollin-3 (Dsc3).

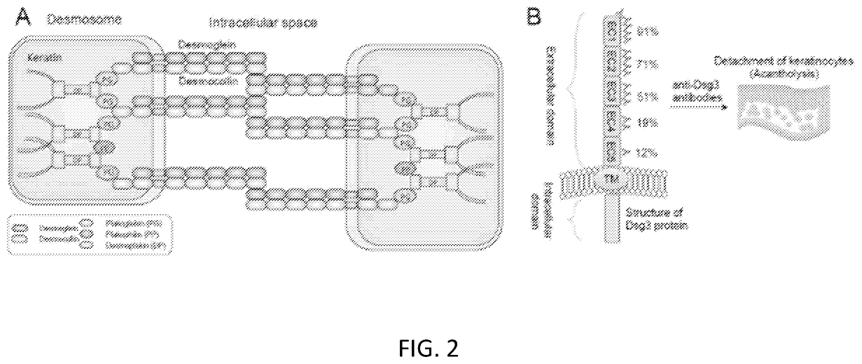

In some embodiments the regions of the autoreactive antigens of interest in the reagents and methods disclosed herein are parts of the autoreactive antigens that are extracellular (EC) domains, as shown in FIG. 2B and FIG. 13. At least in some embodiments, autoreactive antibodies in the diseases and conditions discussed above can be specific for EC domains of autoreactive antigens. Specific epitopes within the autoreactive antigens to which the autoreactive antibodies bind can be called autoreactive epitopes. Generally, since the interest here is in targeting autoreactive B cells, the relevant epitopes can be B cell epitopes. The B cell autoreactive epitopes of the autoreactive antigens can bind to B cell receptors (BCR) of autoreactive B cells. In some embodiments, the B cells that the reagents and methods disclosed herein are designed to target are autoreactive B cells that have BCRs that can bind to these epitopes, for example from the EC domains of molecules like Dsg1, Dsg2, Dsg3, Dsg4, Dsc1, Dsc2, and Dsc3.

Generally, the autoreactive antigens, as described above, stimulate production of autoreactive antibodies. In some embodiments, the autoreactive antibodies produced can be “pathogenic.” In some embodiments, the autoreactive antibodies produced can be “non-pathogenic.”Pathogenic antibodies generally produce symptoms and/or pathology of an autoimmune disease, like pemphigus. Without wanting to be held to a mechanism, pathogenic antibodies can inhibit a function of the protein to which they bind. In some embodiments, therapies described herein can target B cells that produce pathogenic autoreactive antibodies.

Generally, for some autoimmune blistering diseases, like pemphigus, extracellular (EC) domains of autoreactive antigens (e.g., see FIGS. 2B and 13) can be pathogenic or non-pathogenic. In some embodiments, EC domains of desmogleins and/or desmocollins can be pathogenic or non-pathogenic. In some embodiments, EC1, EC2 and/or EC4 domains of Dsg1, Dsg2, Dsg3 and/or Dsg4 can be pathogenic. In some embodiments, EC1, EC2 and/or EC4 domains of Dsg3 and Dsg1 can be pathogenic. While not wishing to be bound by theory, inclusion of non-pathogenic or less pathogenic EC domains in a therapeutic along with pathogenic EC domains is not thought to affect efficacy of the therapeutics disclosed herein.

While not wishing to be bound by theory, levels of immunogenicity of an EC domain (e.g., see the percent of pemphigus patients having autoreactive antibodies to EC domains in FIG. 2B) may or may not be highly correlative with pathogenicity.

### Depleting Autoreactive B Cells

This disclosure describes multiple approaches for therapy in animals, including humans, having an autoimmune disease or condition. The reagents and methods disclosed herein can target and deplete autoreactive B cells in the animals.

In some embodiments, a reagent used to deplete autoreactive B cells can be a fusion protein. The fusion proteins can have separate domains or regions. In some embodiments, a fusion protein can contain one or more autoreactive epitopes that bind to B cell receptors (BCRs) on the surface of B cells reactive with the autoreactive epitope. In some embodiments, the fusion proteins can also contain a domain that can bind to an effector cell. In some embodiments, the domain that can bind to an effector cell can be an Fc region of an antibody. These therapeutics can be called Fc-mediated therapeutics. In some embodiments, the domain that can bind to an effector cell can be an antibody that binds to an effector cell. These therapeutics can be called BCR-effector cell engagers. In embodiments where the antibody can bind to a T cell, the therapeutics can be called BCR-T cell engagers.

In some embodiments, the fusion proteins can have a domain that includes one or more autoreactive B cell epitopes. The autoreactive B cell epitopes can be from any of the autoreactive antigens described above and, in some embodiments, are from EC domains of the autoreactive antigens. Within the context of a fusion protein, the autoreactive B cell epitopes are configured such that they can bind to a BCR of a target autoreactive B cell. In some embodiments, the autoreactive B cell epitope can be located on an end of the fusion protein. In some examples, the B cell epitope can be located at an N-terminal end of the fusion protein.

In some embodiments, “knob-into-hole” strategies can be used to produce Fc-mediated therapeutics that include multiple epitopes, including multiple autoreactive B cell epitopes. Knobs-into-holes is an approach that alters separate C_{H}3 domains from, for example, two IgG heavy chains (C_{H}3 domains contain part of the Fc region) to contain “knobs” or complementary “holes”, such that heterodimerization of the separate C_{H}3 domains is favored (Ridgway, John B B, Leonard G. Presta, and Paul Carter. “‘Knobs-into-holes’ engineering of antibody C_{H}3 domains for heavy chain heterodimerization.” Protein Engineering, Design and Selection 9.7 (1996): 617-621). In some embodiments, two recombinant polypeptides are designed. The first polypeptide can contain a first autoreactive antigen (e.g., one or more EC domains) and one of the “complementary” C_{H}3 domains. The second polypeptide can contain a second autoreactive antigen and the other of the “complementary” C_{H}3 domains. When the first polypeptide is contacted with the second polypeptide, the two C_{H}3 domains preferentially heterodimerize and disulfide bonds are formed between the polypeptides. Homodimerization is not favored. The heterodimerized molecule generally contains a functional Fc region and the two separate autoreactive antigens. In some embodiments, heterodimerized molecules can contain more than two antigens.

In some embodiments, the separate antigens in a “knob-into-hole” molecule can be from different autoreactive antigens. In some embodiments, the separate antigens can contain different epitopes from the same autoreactive antigen. In some embodiments, the separate antigens can be from Dsg3 and/or Dsg1. In some embodiments, the separate antigens can be from different extracellular (EC) domains of a desmoglein. In some embodiments, the separate antigens can be selected from EC1, EC2, EC3, EC4 and EC5 of a desmoglein molecule. For example, the separate antigens can be from EC1 and EC2.

In some embodiments, the autoreactive B cell epitopes can come from the EC1, EC2 and/or EC4 domains of desmosomal cadherins (see Cho, Alice, et al. “Single-cell analysis suggests that ongoing affinity maturation drives the emergence of pemphigus vulgaris autoimmune disease. “Cell reports” 28.4 (2019): 909-922). In some embodiments, EC3 can be included. In some embodiments, the autoreactive B cell epitopes come from the EC1, EC2 and/or EC4 domains of Dsg3. In some embodiments, the autoreactive B cell epitopes come from the EC1 and/or EC2 domains of Dsg3. In some embodiments, a single fusion protein may have multiple EC domains (e.g., two or more of EC1, EC2 and EC3; e.g., EC1-EC4) from one or more desmogleins. In some embodiments, separate fusion proteins, each containing one EC1, EC2, EC3 or EC4 domains of Dsg3 may be combined into a composition and used for therapy in a patient.

While not wishing to be bound by theory, a therapeutic may be designed to contain EC domains corresponding to those that both bind to autoreactive antibodies in a patient, as well as EC domains corresponding to those for which autoreactive antibodies are not currently detected in the patient. In a phenomenon known in the art as “epitope spreading,” some patients have autoreactive antibodies reactive with one epitope on an antigen, but not to other epitopes on the antigen. Over time, however, the patients can develop autoreactive antibodies to one or more of the epitopes which did not initially stimulate autoreactive antibodies. Inclusion in the therapeutics disclosed herein, of the epitopes to which autoreactive antibodies later emerge can prevent or lessen symptoms of the disease caused by the later-arising antibodies. In some embodiments, in a patient that has autoreactive antibodies initially only against EC5 of Dsg3 or Dsg1, administration of a therapeutic containing EC1, EC2, EC4, as well as EC5, can be beneficial to the patient.

In some embodiments, the autoreactive B cell epitopes can come from plakins (e.g., envoplakin, periplakin, desmoplakin I, desmoplakin II, epiplakin, plectin, BP230), cadherins ((Dsg3, Dsg1, Dsc1, Dsc2, Dsc3, α2-macroglobulin-like molecules), BP180 (e.g., NC16A, LABD97, LAD-1 domains or C-terminal epitopes), laminin 332, α6β4 integrin, collagen VII, Laminin gamma1 (p200), Type VII collagen, epidermal transglutaminase, tissue transglutaminase, endomysium or deamidated gliadin.

Generally, the autoreactive antibodies can be any class of antibody (IgA, IgD, IgE, IgG or IgM). In some instances, the autoreactive antibodies can be IgA or IgG.

In some embodiments, instead of autoreactive B cell epitopes, allergens, parts of allergens, or molecules that can mimic allergens can be part of the Fc-mediated therapeutics or BCR-T cell engagers disclosed herein. Many different allergens exist. Nonlimiting examples of allergens may be from drugs, foods, insects (e.g., insect stings, dust mites), latex, molds, pets/animals (e.g., dander) and pollen. Allergens can include urushiol, as found in plants like poison ivy, eastern poison oak, western poison oak, poison sumac, and the like. In some embodiments, the allergen can be birch pollen allergens, like Bet v 1. In some embodiments, the allergens/parts of allergens can be proteins, polypeptides or polypeptides.

In some examples, the allergies can be type I hypersensitivities. For example, the allergies can be mediated by IgE. B-cells can produce the IgE. The IgE can then bind to IgE-specific receptors found, for example, on mast cells, basophils, and the like, which cells can release mediators of allergies, like cytokines, vasoactive amines (e.g., histamine), proteases, prostaglandins, leukotrienes, and the like. In some embodiments, allergens that are part of Fc-mediated therapeutics or BCR-T cell engagers can bind and deplete B-cells that produce IgE. In some examples, the allergies can be type II, type III or type IV hypersensitivities.

In some embodiments, the fusion proteins can have a domain that binds to an effector cell. In fusion proteins having an autoreactive B cell epitope and an effector-cell binding domain, the fusion protein can be configured such that binding of the autoreactive B cell epitope to a BCR on a B cell, co-localizes effector cells bound to the effector-cell binding domain of the fusion protein in proximity to the B cells such that the effector cells can act on the B cell and deplete the B cell. In some embodiments, the domain that binds to an effector cell can be a Fc portion of an antibody. In some embodiments, the domain that binds to an effector cell can be an antibody specific for the effector cell.

In some embodiments, the effector-cell binding domain can be located on an end of the fusion protein. In some examples, the effector-cell binding domain can be located at a C-terminal end of the fusion protein.

In some embodiments, the effector cells to which the effector-cell binding domain of a fusion protein can bind can be a phagocytic cell or phagocyte. The phagocyte may include so-called “professional” and “non-professional” phagocytes. In some embodiments, professional phagocytes can include monocytes, macrophages, neutrophils, dendritic cells and mast cells. In some embodiments, non-professional phagocytes can include certain T cells (e.g., cytotoxic T cells) and natural killer (NK) cells.

In some embodiments, the domain of the fusion protein that binds to an effector cell can be an Fc portion or domain of an antibody. Generally, any Fc portion may be used in the reagents and methods disclosed herein. In some embodiments, any Fc portion that can bind to an effector cell (e.g., NK cell, macrophage, and the like) can be used. In some embodiments, any Fc portion that can mediate depletion of the target pathogenic B cells (e.g., autoreactive B cells) can be used. The Fc portion can bind to a cell that has an Fc receptor (FcR). The Fc portion can bind to an FcR on the cell. In some embodiments, the FcR can be an Fc-gamma receptor (FcγR), an Fc-alpha receptor (FcαR) or an Fc-epsilon receptor (FcεR). In some embodiments, the FcγR can be an FcγRI, FcγRII or FcγRIII. In the context of a fusion protein, the Fc portion of an antibody is configured such that it can bind to an Fc receptor (FcR) on a cell.

In some embodiments, the Fc portion or domain of an antibody used herein may be modified. In some embodiments, the modification or modifications can affect binding of the Fc region to Fc receptors on various cells. In some embodiments, the modifications can affect function of cells to which the Fc regions bind. In some embodiments, the Fc portion or domain can be heterogeneously modified at a conserved N-glycosylation site on the Fc domain. In some embodiments, a complex, biantennary glycan can be attached at the conserved N-glycosylation site on the Fc domain (see Li, Tiezheng, et al. “Modulating IgG effector function by Fc glycan engineering.” Proceedings of the National Academy of Sciences 114.13 (2017): 3485-3490). Generally, any type of Fc domain modification can be used, as long as the modified Fc domain can bind to an effector cell (e.g., NK cell, macrophage, and the like). Generally, any type of Fc domain modification can be used, as long as the modified Fc domain can mediate depletion of the target pathogenic B cells (e.g., autoreactive B cells).

Within the fusion protein, the Fc portion can also be configured such that an effector cell to which the Fc portion binds is co-localized in proximity to the autoreactive B cell to which the autoreactive B cell epitope of the fusion protein binds. Effector cells that have an FcR, and to which the Fc portion of the fusion protein can bind, can include for example, neutrophils, monocytes, macrophages, mast cells and dendritic cells.

In some embodiments, the domain of the fusion protein that binds to an effector cell can be an antibody that binds to an effector cell. Herein, fusion proteins that contain an autoreactive antigen and an antibody that binds to an effector cell can be called BCR-effector cell engagers. In some embodiments, the antibody can be an antibody that binds T cells (e.g., an anti-CD3 antibody) or specific types of T cells. In some embodiments, the antibody can be an antibody that binds cytotoxic T cells (e.g., an anti-CD8 antibody) or an antibody that binds helper T cells (e.g., an anti-CD4 antibody). In some embodiments, the antibody can be an antibody that binds, for example, macrophages, natural killer (NK) cells or other phagocytes. In some examples, an antibody or combination of antibodies that bind NK cells can be used. In some examples, an anti-CD56 antibody may be used. In some embodiments, the antibody can be a single-chain variable fragment (scFv).

Within the fusion protein, the antibody that binds to an effector cell is configured such that it can bind to the specific effector cell. In some embodiments, the antibody can bind to one or more desired effectors cells, but not bind or bind less efficiently to other cells. Within the fusion protein, the antibody that binds to an effector cell is also configured such that an effector cell to which the antibody binds is co-localized in proximity to the autoreactive B cell to which the autoreactive B cell epitope of the fusion protein binds. This co-localization facilitates the ability of the effector cell to interact with/act on the autoreactive B cell.

The fusion proteins disclosed herein can additionally have a spacer or linker. Generally, a spacer can be located in the fusion protein between the autoreactive B cell epitope domain and the domain that binds to an effector cell. The spacer can facilitate the separate domains within a fusion protein to each perform their desired function. For example, a spacer located between a domain of a fusion protein that includes an autoreactive B cell epitope and a domain that binds to an effector cell, can provide for the autoreactive B cell epitope to bind to a BCR on an autoreactive B cell while, simultaneously, the effector-cell binding domain can bind to and co-localize the effector cell in proximity to the autoreactive B cell. The spacer can provide for the co-localized effector cell to deplete the autoreactive B cell.

In some embodiments, the spacer can be an amino acid spacer. In some embodiments, the spacer can be between about 2 and 50 amino acids in length. In some embodiments, the spacer can be between about 5 and 45, 6 and 40, 7 and 35, 8 and 30, 9 and 25, or 10 and 20 amino acids in length. In some embodiments, the spacer can be a flexible spacer that can increase flexibility of the fusion protein.

In some embodiments, flexible peptide spacers can include small, polar (e.g., Ser, Thr) or non-polar (e.g., Gly) amino acids. The flexible peptide spacers can have sequences of Gly and Ser residues (e.g., a “GS” linker). An example GS linker amino acid sequence can include (Gly-Gly-Gly-Gly-Ser)ₙ. An example peptide spacer can be (Gly-Gly-Gly-Gly-Ser) 3. Other types of flexible spacers can include KESGSVSSEQLAQFRSLD, EGKSSGSGSESKST, (Gly) 8, GSAGSAAGSGEF and (GGGGS)+ (Chen, Xiaoying, Jennica L. Zaro, and Wei-Chiang Shen. “Fusion protein linkers: property, design and functionality.” Advanced drug delivery reviews 65.10 (2013): 1357-1369). Other example types of spacers can be rigid spacers or cleavable spacers.

In some embodiments of the fusion protein, the domains that include an autoreactive B cell epitope and the effector-cell binding domain can be connected to one another using various chemistries. In some embodiments, so-called “click” chemistry can be used. In some embodiments, sortase enzymes can be used.

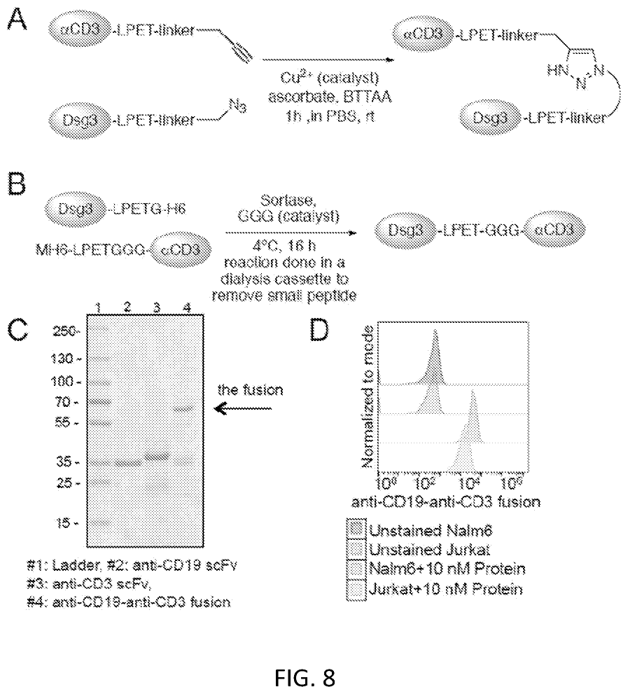

In some embodiments, the domains can be connected using C-to-C fusion chemistry. For example, sortase can be used to install click “handles” at C-termini of the autoreactive C cell epitope domain and the effector-cell binding domains. The two modified domains can be “clicked” together using, for example, using azide-alkyne click chemistry, producing the C-to-C fusion (FIG. 8A).

In some embodiments, the domains can be connected using N-to-C fusion chemistry. For example, using the reversibility of the sortase enzyme, a triglycine molecule (Gly-Gly-Gly) can be used to catalyze formation N-to-C fusion of the two domains. One of the domains (first domain) can be modified to have a Leu-Pro-Glu-Thr-Gly at its C terminus. The other domain (second domain) can be modified to have a Leu-Pro-Glu-Thr-Gly-Gly-Gly at its N terminus. By reacting the two modified domains in presence of triglycine and sortase, the Leu-Pro-Glu-Thr segment is removed from the second modified domain and converted to Gly3. The Gly3-second domain can then react with the modified first domain to form the fusion protein (FIG. 8B).

In some embodiments, the fusion proteins disclosed here can include additional moieties/domains, like a therapeutic moiety, an imaging moiety, a capturing moiety, and combinations thereof. In some embodiments, these moieties can be added to the fusion proteins using click chemistry and/or sortase enzymes. In some embodiments, a therapeutic moiety can include a cytotoxin, a toxin, a radiotherapeutic agent, a T cell-engaging moiety, a natural killer (NK) cell engaging moiety, or a combination thereof. In some embodiments, a therapeutic moiety can include IL-2 or a molecule having IL-1 activity. In some embodiments, an imaging moiety can include a fluorophore, a radioisotope, or a combination thereof. In some embodiments, a capturing moiety can include a hydrophilic protein, a bacterial transpeptidase enzyme, a GST tag, a His-Tag, polyethylene glycol (PEG), or a combination thereof.

Regarding the fusion proteins disclosed herein and individual domains of those proteins (e.g., autoreactive epitopes, Fc regions, antibodies that bind effector cells), one of skill in the art will readily recognize that individual substitutions, deletions or additions of amino acids to peptide, polypeptide, or protein sequence, or to nucleotides of a nucleic acid sequence, which alters, adds, deletes, or substitutes a single amino acid or a small percentage of amino acids in the encoded sequence is collectively referred to herein as a “conservatively modified variant”. In some embodiments the alteration results in the substitution of an amino acid with a chemically similar amino acid. Conservative substitution tables providing functionally similar amino acids are well known in the art. Such conservatively modified variants of the Fc domains or antibodies disclosed herein can exhibit increased effector-cell binding in comparison to unmodified sequences.

For example, a “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is replaced with another amino acid residue from the same side chain family. In another embodiment, a string of amino acids can be replaced with a structurally similar string that differs in order and/or composition of side chain family members.

In some embodiments, the autoreactive epitopes, Fc regions, or antibodies that bind effector cells can have a specified percentage identity or similarity to the amino acid or nucleotide sequences of the autoreactive epitopes, Fc regions, or effector-cell-binding antibodies described herein. For example, “homology” or “identity” or “similarity” refers to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous at that position. A degree of homology between sequences is a function of the number of matching or homologous positions shared by the sequences. For example, the molecules described herein can have 60%, 70%, 75%, 80%, 85%, 90%, 91%, 92%, 93%, 94%, 95%, 96%, 97%, 98%, 99%, or higher amino acid sequence identity when compared to a specified region or the full length of any one of the molecules described herein. For example, the molecules described herein can have 60%, 70%, 75%, 80%, 85%, 90%, 91%, 92%, 93%, 94%, 95%, 96%, 97%, 98%, 99%, or higher nucleic acid identity when compared to a specified region or the full length of any one of the antibodies described herein. Sequence identity or similarity to the nucleic acids and proteins of the present invention can be determined by sequence comparison and/or alignment by methods known in the art, for example, using software programs known in the art, such as those described in Ausubel et al. eds. (2007) Current Protocols in Molecular Biology. For example, sequence comparison algorithms (i.e. BLAST or BLAST 2.0), manual alignment or visual inspection can be utilized to determine percent sequence identity or similarity for the nucleic acids and proteins of the present invention.

### Therapeutic Preparations

Aspects of the invention are drawn towards therapeutic preparations. As used herein, the term “therapeutic preparation” can refer to any compound or composition that can be used or administered for therapeutic effects. As used herein, the term “therapeutic effects” can refer to effects sufficient to result in amelioration of symptoms, e.g., treatment, healing, prevention or amelioration of the relevant medical condition, or an increase in rate of treatment, healing, prevention or amelioration of such conditions.

Embodiments as described herein can be administered to a subject in the form of a pharmaceutical composition or therapeutic preparation prepared for the intended route of administration. Such compositions and preparations can comprise, for example, the active ingredient(s) and a pharmaceutically acceptable carrier. Such compositions and preparations can be in a form adapted to oral, subcutaneous, parenteral (such as, intravenous, intraperitoneal), intramuscular, rectal, epidural, intratracheal, intranasal, dermal, vaginal, buccal, ocularly, or pulmonary administration, such as in a form adapted for administration by a peripheral route or is suitable for oral administration or suitable for parenteral administration. Other routes of administration are subcutaneous, intraperitoneal and intravenous, and such compositions can be prepared in a manner well-known to the person skilled in the art, e.g., as generally described in “Remington's Pharmaceutical Sciences”, 17. Ed. Alfonso R. Gennaro (Ed.), Mark Publishing Company, Easton, Pa., U.S.A., 1985 and more recent editions and in the monographs in the “Drugs and the Pharmaceutical Sciences” series, Marcel Dekker. The compositions and preparations can appear in conventional forms, for example, solutions and suspensions for injection, capsules and tablets, in the form of enteric formulations, e.g., as disclosed in U.S. Pat. No. 5,350,741, and for oral administration.

Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition can be sterile and can be fluid to the extent that easy syringeability exists. In embodiments, it can be stable under the conditions of manufacture and storage and can be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyethylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the compound in the required amount in an appropriate solvent with one or a combination of ingredients enumerated herein, as required, followed by filtered sterilization. Generally, dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Oral formula of the drug can be administered once a day, twice a day, three times a day, or four times a day, for example, depending on the half-life of the drug.

Pharmaceutically compatible binding agents, and/or adjuvant materials can be included as part of the composition administered to a subject. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel® (sodium starch glycolate), or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In embodiments, administering can comprise the placement of a pharmaceutical composition into a subject by a method or route which results in at least partial localization of the composition at a desired site such that desired effect is produced.

For example, the pharmaceutical composition can be administered by bolus injection or by infusion. A bolus injection can refer to a route of administration in which a syringe is connected to the IV access device and the medication is injected directly into the subject. The term “infusion” can refer to an intravascular injection.

Embodiments as described herein can be administered to a subject one time (e.g., as a single injection, bolus, or deposition). Alternatively, administration can be once or twice daily to a subject for a period of time, such as from about 2 weeks to about 28 days. Administration can continue for up to one year. In embodiments, administration can continue for the life of the subject. It can also be administered once or twice daily to a subject for period of 1, 2, 3, 4, 5, 6, 7, 8, 9, 10, 11, 12 times per year, or a combination thereof.

In embodiments, compositions as described herein can be administered to a subject chronically. “Chronic administration” can refer to administration in a continuous manner, such as to maintain the therapeutic effect (activity) over a prolonged period of time.

A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

A therapeutically effective amount of a reagent or therapeutic composition of the invention can be the amount needed to achieve a therapeutic objective. As noted herein, this can be a binding interaction between the reagent or therapeutic composition and its target that, in certain cases, interferes with the functioning of the target. The amount required to be administered will furthermore depend on the binding affinity of the reagent or therapeutic composition for its specific target and will also depend on the rate at which an administered reagent or therapeutic composition is depleted from the free volume other subject to which it is administered. The dosage administered to a subject (e.g., a patient) of the binding polypeptides described herein is typically 0.1 mg/kg to 100 mg/kg of the patient's body weight, between 0.1 mg/kg and 20 mg/kg of the patient's body weight, or 1 mg/kg to 10 mg/kg of the patient's body weight. Human antibodies have a longer half-life within the human body than antibodies from other species due to the immune response to the foreign polypeptides. Thus, lower dosages of human antibodies and less frequent administration is often possible. Further, the dosage and frequency of administration of reagent or therapeutic composition of the disclosure may be reduced by enhancing uptake and tissue penetration (e.g., into the brain) of the antibodies by modifications such as, for example, lipidation. Common ranges for therapeutically effective dosing of an antibody or antibody fragment of the invention can be, by way of nonlimiting example, from about 0.1 mg/kg body weight to about 50 mg/kg body weight. Common dosing frequencies can range, for example, from twice daily to once a week.

Where fragments (e.g., antibody fragments) are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al, Proc. Natl. Acad. Sci. USA, 90:7889-7893 (1993)). The formulation can also contain more than one active compound as necessary for the particular indication being treated, for example, those with complementary activities that do not adversely affect each other. Alternatively, or in addition, the composition can comprise an agent that enhances its function, such as, for example, a cytotoxic agent, cytokine (e.g., IL-15), chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions. Sustained-released preparations can be prepared.

The pharmaceutical or therapeutic carrier or diluent employed can be a conventional solid or liquid carrier. Nonlimiting examples of solid carriers are lactose, terra alba, sucrose, cyclodextrin, talc, gelatin, agar, pectin, acacia, magnesium stearate, stearic acid or lower alkyl ethers of cellulose. Nonlimiting examples of liquid carriers are syrup, peanut oil, olive oil, phospholipids, fatty acids, fatty acid amines, polyoxyethylene and water. Similarly, the carrier or diluent can include any sustained release material known in the art, such as glyceryl monostearate or glyceryl distearate, alone or mixed with a wax.

When a solid carrier is used for oral administration, the preparation can be tableted, placed in a hard gelatin capsule in powder or pellet form or it can be in the form of a troche or lozenge. The amount of solid carrier will vary widely but will usually be from about 25 mg to about 1 g.

When a liquid carrier is used, the preparation can be in the form of a syrup, emulsion, soft gelatin capsule or sterile injectable liquid such as an aqueous or non-aqueous liquid suspension or solution.

The composition and/or preparation can also be in a form suited for local or systemic injection or infusion and can, as such, be formulated with sterile water or an isotonic saline or glucose solution. The compositions can be in a form adapted for peripheral administration only, with the exception of centrally administrable forms. The compositions and/or preparations can be in a form adapted for central administration.

The compositions and/or preparations can be sterilized by conventional sterilization techniques which are well known in the art. The resulting aqueous solutions can be packaged for use or filtered under aseptic conditions and lyophilized, the lyophilized preparation being combined with the sterile aqueous solution prior to administration. The compositions and/or preparations can contain pharmaceutically and/or therapeutically acceptable auxiliary substances as required to approximate physiological conditions, such as buffering agents, tonicity adjusting agents and the like, for instance sodium acetate, sodium lactate, sodium chloride, potassium chloride, calcium chloride, etc.

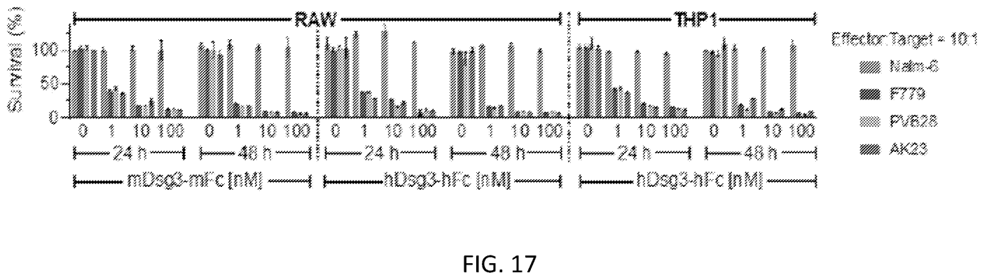

Patients with autoimmune diseases, including an autoimmune blistering disease like pemphigus, have autoreactive antibodies circulating with their blood. The therapeutics described herein contain autoreactive epitopes to which the autoreactive antibodies in the patient could possibly bind. A potential concern can be that the autoreactive antibodies could bind to and/or inactivate the therapeutic after it has been administered to a patient. However, data presented herein (e.g., see Example 7, FIGS. 17 & 18) show that the autoreactive antibodies do not decrease the effectiveness of these therapeutics. In some embodiments, effectiveness of the therapeutics disclosed herein are not decreased or are minimally decreased by the presence of autoreactive antibodies in the patient to which the therapeutic is administered.

### Nucleic Acids, Vectors and Cells Expressing Fusion Proteins

Also disclosed are nucleic acids encoding all or part of the fusion proteins described herein. Also disclosed are various vectors (e.g., plasmids, viral, and the like) that include the nucleic acids. Also disclosed are various cells (e.g., prokaryotic, eukaryotic) that contain nucleic acids or vectors and can express the fusion proteins.

### EXAMPLES

Examples are provided herein to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Developing Non-Immunosuppressive, Immune-Based Therapeutics for Targeted Treatment of Autoimmune Diseases

Pemphigus vulgaris (PV) is a devastating autoimmune blistering disease, which can be characterized by painful blistering of the skin and mucosa. PV is caused by production of autoantibodies against desmoglein 3 (Dsg3), with or without antibodies against Dsg1, resulting in epithelial detachment (acantholysis) There is no cure for PV; treatment strategies can include systemic steroids and immunosuppressive drugs. While anti-CD20 antibodies (e.g., rituximab) have become first line option for moderate-to-severe disease, it can cause off-target immunosuppressive effects including increased risk of infection albeit lower than traditional immunosuppressive agents and a high risk of relapse after treatment. Relapse in PV can be due to expansion of the same autoreactive cells, not depleted during initial treatment; thus, complete depletion of autoreactive B cells can be a cure. Neonatal Fc receptor and Bruton's Tyrosine Kinase inhibitors, currently in clinical trials, cannot circumvent the off-target immunosuppressive effects. Early-stage cell-based therapies such as autologous polyclonal regulatory T cells and chimeric autoantigen receptor (CAAR) T cells, if proven effective, cannot be globally immunosuppressive. However, cell-based therapies must be personalized, have specific requirements for manufacturing, storage and transport, which is not feasible in resource-poor areas.

There is no cure for PV. The treatment described herein can be curative and can be precise as it utilizes the specificity of the existing adaptive immune machinery. The treatment proposed are not expected to be globally immunosuppressive, thus circumventing the major side-effects of existing treatments. Additionally, it eliminates the need for personalization as the target for PV is universal. Importantly, the current project provides a platform for the treatment of not only PV but can be modified to be used in all antibody-driven autoimmune diseases with known antigens, which are predominantly treated with traditional nonspecific, immunosuppressive treatments.

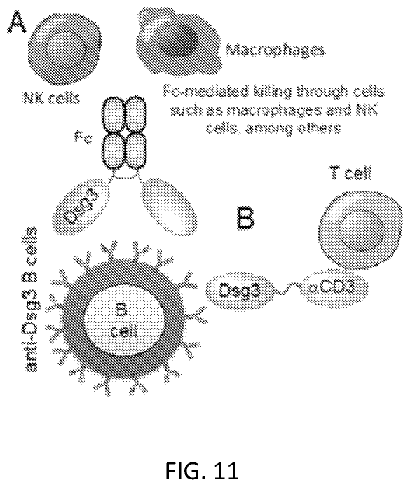

Based on existing oncologic treatments, we designed two approaches to precisely deplete autoreactive anti-Dsg3 B cells: 1) Fc-mediated, and 2) BCR-T cell engagement (see FIG. 11).

Fc-mediated approach: this approach is analogous to the use of rituximab in PV, with major advantage of targeting anti-Dsg3 B cells.

BCR-T cell engager approach: The Dsg3-anti-CD3e construct can establish the molecular clustering and formation of the immunologic synapses between the target anti-Dsg3 B cells and the T cells, which will consequently result in the proliferation and activation of T cells to specifically kill the targeted anti-Dsg3 B cells, leaving other B cells intact. Unlike in oncological applications, cytokine release syndrome is not expected in treatment of autoimmune diseases, given the burden of autoreactive B cell is lower than for malignant clones.

One strategy of making Dsg3-Fc can be safe and can replace the use of Rituximab. The other approach provides an attractive alternative option for patients who fail to respond to available treatments. Therefore, this project has the potential to transform the treatment of not only PV, but open an avenue for developing treatments for several autoantibody/immune-complex mediated diseases to known antigenic targets, ranging from other autoimmune blistering diseases (e.g., bullous pemphigoid, mucous membrane pemphigoid, epidermolysis bullosa acquisita, linear IgA bullous dermatosis), subtypes of lupus, scleroderma, Goodpasture's disease, Graves' disease, to immune-mediated vasculitis. Therefore, without wishing to be bound by theory, the therapeutics disclosed herein can be considered a ground-breaking addition to the therapeutic toolbox for autoimmune diseases.

Our therapeutic can be used concurrently with other drugs that lower anti-Dsg3 titer. Further, reassuringly, monoclonal and polyclonal anti-Dsg3 IgG was found not to have a significant effect on the killing abilities of CAAR T cells specific for Dsg3-autoantibody producing B cells.

### Example 2

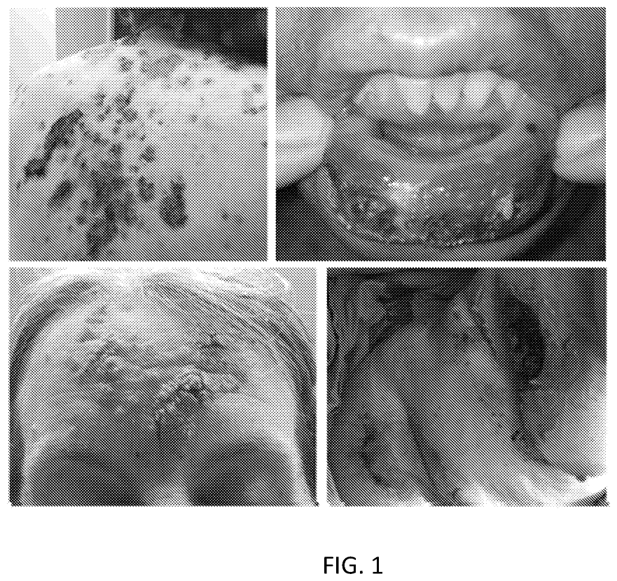

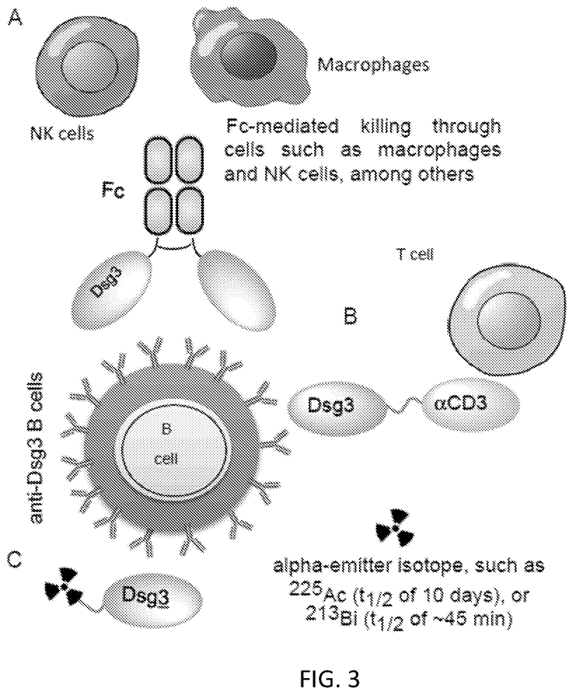

Pemphigus vulgaris (PV) is an autoimmune blistering disease, characterized by painful blistering of the skin and mucosa (FIG. 1). PV is caused by production of autoantibodies against desmoglein 3 (Dsg3), with or without antibodies against Dsg1, resulting in epithelial detachment (acantholysis) (FIG. 2 panels A-B). There is no cure for PV; treatment strategies can include systemic steroids and immunosuppressive drugs. While anti-CD20 antibodies against B cells (e.g., rituximab) have become a first line option for moderate-to severe disease, it is associated with off-target immunosuppressive effects including increased risk of infection albeit lower than traditional immunosuppressive agents and a high risk of relapse after treatment. Neonatal Fc receptor and Bruton's Tyrosine Kinase inhibitors, currently in clinical trials, cannot circumvent the off-target immunosuppressive effects. Early-stage cell-based therapies such as autologous polyclonal regulatory T cells 14 and chimeric autoantigen receptor (CAAR) T cells 16, if proven effective, cannot be globally immunosuppressive. However, cell-based therapies can be personalized, have specific requirements for manufacturing, storage and transport, which can or cannot be feasible in resource-poor areas. Relapse in PV is typically due to expansion of the same autoreactive cells, not depleted during initial treatment; complete depletion of autoreactive B cells will offer a potential cure. Three approaches were designed to precisely deplete autoreactive anti-D sg3 B cells: 1) Fc-mediated, 2) BCR-T cell engagement and 3) radiotherapy approaches (FIG. 3 panels A-C). The current project provides a platform for the treatment of not only PV but potentially several antibody-driven autoimmune diseases with known antigens.

Fc-mediated: this approach is analogous to the use of rituximab in PV, in the sense that B cells are targeted, with a major advantage of targeting anti-Dsg3 B cells-only.

BCR-T cell engager: The Dsg3-anti-CD3e construct can establish the molecular clustering and formation of the immunological synapses between the target anti-Dsg3 B cells and the T cells, which can consequently result in the proliferation and activation of T cells to specifically kill the targeted anti-Dsg3 B cells, leaving other B cells intact. Unlike in oncological applications, cytokine release syndrome is not expected in treatment of autoimmune diseases, given the burden of autoreactive B cell is lower than for malignant clones.

Radiotherapy: In clinical practice, the short range of alpha particles in tissue (<0.1 mm), allows for selective killing of the targeted cells without damaging neighboring tissue; the killing is independent of oxygenation status, cell cycle, cell internalization and prior treatments. Radiotherapy can therefore offer an attractive alternative for patients who are resistant to traditional therapy, Rituximab, or our Dsg3-Fc and Dsg3-anti-CD3 approaches.

### Approach.

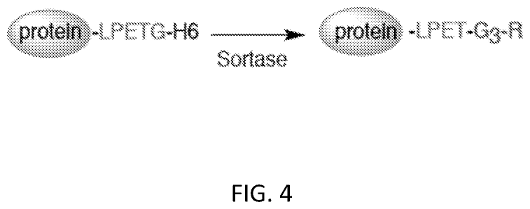

Site-specific modification of proteins. Sortase is a bacterial transpeptidase enzyme that recognizes an “LPETG” motif and cleaves the bond between threonine and glycine to form a thio-acyl intermediate, which is substituted with a triglycine containing probe (GGG-Probe). Thereby, sortase can convert a “protein-LPETG” to “protein-LPET-GGG-probe” product, where the probe can be any biomolecule of interest (FIG. 4), rapidly, with near complete yield. We and others have used sortase to install fluorophores, polymers, radioisotopes or a secondary protein into biomolecules. We can use sortase to install chelators and fluorophores on Dsg3 protein and antibody-fragments and to make N-to-C and C-to-C fusion proteins that are otherwise difficult or impossible to generate using traditional genetic approaches.

Immunogenicity of Dsg3 ectodomains. Studies on PV patients have shown that 91%, 71%, 51%, 19% and 12% of patients have autoantibodies against ectodomains EC1, EC2, EC3, EC4 and EC5, respectively (FIG. 2 panel B, FIG. 13). We used Dsg3-EC1-3, as it covers the majority of the physiological anti-Dsg3 antibodies in PV patients and its production is less complex than the full-size EC1-5 construct. We can also use EC1-4 and 1-5 constructs.

Plasmid and cell lines. We used three cell lines: 1) AK23 hybridoma cells, which produces anti-mouse Dsg3 antibodies, which induces PV in mice. The AK23 antibody recognizes the EC1 domain and cross-reacts with human Dsg3-EC1; 2 and 3) Nalm-6 F779 and Nalm-6 PVB-28 cancer cell lines. Nalm-6 is a B cell precursor leukemia cell line, and F779 and PVB-28 are the cells engineered to express patient-derived anti-Dsg3 antibodies that recognize Dsg3-EC1 and Dsg3-EC2 domains, respectively. Furthermore, we have obtained the plasmid for an anti-human Dsg3 antibody (P3F3), identified from a PV patient. The P3F3 antibody is an IgG1 antibody that binds specifically to human Dsg3-EC1 domain, inducing strong acantholysis when added to human keratinocytes.

Animal studies. The efficacy of the therapeutics can be tested in two validated PV mouse models:

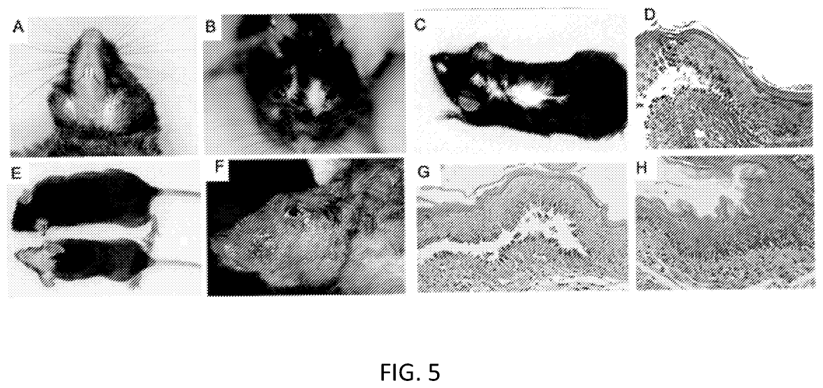

Passive Transfer Model. In this model, we produced a passive monoclonal pemphigus mouse disease model by implanting an established pemphigus-inducing hybridoma cell line (AK23; already in hand). As previously described, we injected ˜5-10 million AK23 hybridoma cells, which secrete anti-Dsg3 antibodies that cause the pemphigus phenotype, or unrelated hybridoma cells as control, into eight-week-old C57BL/6J mice that were primed with 2,6,10,14-tetramethyl-pentadecane (pristane) (n=10 each group). Animals were assigned in all experiments to treatment/control groups based on an urn randomization method. We tested for establishment of the disease by analyzing serum anti-Dsg3 titers (by standard ELISA on Day 3 and Day 10), and skin blisters (assessed by blinded investigators on Day 2, 5 and 10). Based on the ELISA data, time point for the tissue harvest was determined to observe acantholysis (by H&E staining). The peak disease time can be around Day 7; the mice can exhibit hair loss, weight loss, and have blistering in hair follicles and oral mucosa along with AK23 autoantibodies produced by AK23 cells binding to epidermal keratinocytes (FIG. 5 panels A-D) with physiologic anti-Dsg3 IgG levels (300-400 RU/ml).

Active Transfer Model. The AK23 model has advantages, however, treatment can overcome the fast-proliferating hybridoma cells. Therefore, as a second model, we used an active PV immune model that closely mimics the clinical phenotype of human disease, has lower frequency of autoreactive Dsg3-specific B cells that resembles what happens clinically, and finally has a high serum polyclonal autoantibody level. In this model, we developed an active polyclonal pemphigus mouse disease model by first immunizing Dsg3−/− mice (JAX-002911) with recombinant extracellular domain of mouse Dsg3, followed by the transfer of splenocytes into RAG2−/− mice. Transferred cells generally contained T cells and B cells, which can include Dsg3-reactive cells. Similar to observations made in the literature, without wishing to be bound by theory, animals can have stunted growth (FIG. 5 panel E) and develop mucocutaneous erosions with suprabasal acantholysis and blisters (FIG. 5 panels F-H). To assess the clinical index, using ELISA we compared autoantibody levels to physiological levels in PV patients (300-400 RU/ml). When the Dsg3 reactive antibodies equaled or exceeded that level, which (without wishing to be bound by theory) can happen in 3-4 weeks, treatment was initiated. Adoptive transfer of cells from Dsg3-immunized Dsg3+/− animals was used as a control 38 (n=10 mice/group).

### Monitoring.

Side effects: Animals received treatments intravenously via tail-vein along with the proper controls, as described herein. Ten mice were maintained for each treatment from both disease models for at least 6 months after the treatment. Anti-Dsg3 levels were measured by ELISA every 2-3 weeks to test long-lasting remission. Mice were tested by a blinded investigator to check for skin lesions to ensure no disease relapse. Without wishing to be bound by theory, mice can maintain complete remission. The Fc-mediated approach can or cannot show some relapse, as with rituximab, but without wishing to be bound by theory, can respond with retreatment. Considering clinical translation, the minority of patients who cannot respond to the Dsg3-Fc treatment, can have the option of radiotherapeutic and/or the BCR-T cell engager approach.

Infection susceptibility: To test if our therapy globally suppresses the immune system, C57BL/6 mice can be infected with active LCMV-C113. Animals can be injected with AK23 hybridoma 21 days post-infection followed by our treatment strategies according to experimental design described herein. Viral load for infected and control mice can be compared 30 days post-injection. Alternatively, at day 7 post our PV therapy, mice can be challenged orally with Listeria monocytogenes 40 or intravenously with LCMV infection. Microbial load in liver and spleen will be checked 10 days post-infection and compared with infection-only mice. In both experiments, and without wishing to be bound by theory, there will not be any difference in the viral load.

Testing the ability of Dsg3-Fc fusion proteins to mediate killing of anti-Dsg3 B cells.

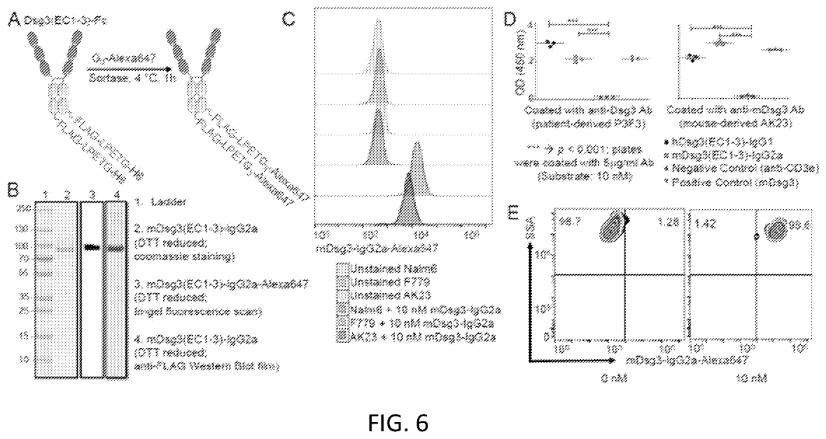

Non-limiting, exemplary results. We have made both mouse and human Fc-fusion proteins: mDsg3-EC1-3-IgG2a (mFc) and hDsg3-EC1-3-IgG1 (hFc). Fc regions from human IgG1 and mouse IgG2a were chosen for their high Fc-mediated killing activities. The proteins were engineered to have a FLAG tag, a sortase recognition motif and a His6 tag at the C-terminus. The FLAG, an 8 amino acid sequence (DYKDDDDK), can be used for Western Blot and flow cytometry analyses, and subsequently for analysis of the construct metabolic fate when used in vivo. The His6 tag was used for purification and the sortase tag was used to site-specifically introduce modifications. We expressed both constructs using the mammalian Expi293F cells, purified via Ni-NTA affinity column, and characterized using SDS-PAGE and western blotting analyses, which confirmed formation of the constructs (FIG. 6 panels A-B). A Gly3-Alexa647 sortase substrate was synthesized and used to site-specifically label the fusion proteins with Alexa Fluor 647. Sortase reaction was performed by mixing the construct (50 μM), the Gly3-Alexa647 substrate (500 μM), and sortase (5 μM) for 1 h, at 4° C. The Alexa647-labeled constructs were purified via size-exclusion column chromatography and the labeling were further confirmed by in-gel fluorescence scan of the constructs (FIG. 6 panel B). To characterize the functionality of the constructs, we used Alexa647-labeled mDsg3-EC1-3-mFc to stain the anti-mDsg3 AK23 hybridoma and anti-hDsg3 F779 cells. The cells were stained with affinity and specificity (at low nM concentration) (FIG. 6 panel C), indicating that the patient derived BCRs on the F779 cells cross-react with mouse Dsg3 protein. Nalm-6 cells, used as control, did not show any binding (FIG. 6 panel C). Similar experiments were performed using the Alexa647-labeled hDsg3-EC1-3-IgG1 (hFc), which indicated that the construct binds to the F779 cells and stained the mouse AK23 cells too (similar to FIG. 6 panels B-C). To further characterize the constructs, we performed ELISA assays by first coating a plate with the AK23 Ab and another plate with P3F3 (anti-human Dsg3). The results further indicated that the human and mouse constructs we made specifically bind to both antibodies (FIG. 6 panel D). P3F3 antibody was made in the Expi293F cells, following manufacturer's protocol. AK23 antibody was purified from the AK23 hybridoma media. Following similar approach, we will generate mouse and human Dsg3-EC1-4 and Dsg3-EC1-5 fused to human and mouse Fc and will fully characterize their binding.

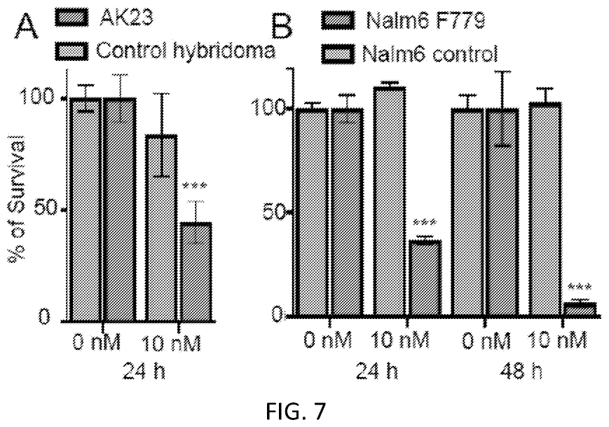

Establishing the Fc-mediated in vitro killing assay: First, we confirmed that the Dsg3-Fc fusion, through its Fc (IgG2a), bound to the Fcγ receptor of mouse macrophages with low nM affinity (FIG. 6 panel E); control Nalm6 cells remained unstained. To determine whether the fusion protein can result in Fc-mediated killing, F779 or AK23 target cells, co-cultured with mouse RAW264.7 macrophage cells, were treated with the mDsg3-mFc fusion protein. A 10 nM dose resulted in 65% and 60% killing of the F779 and AK23 cells, respectively, after 24 hours, with no killing of either Nalm6 or hybridoma control cells. At a 48-hour timepoint, we observed 94% killing of F779 cells with no change in the control Nalm-6 cells (FIG. 7). AK23 hybridoma cells release anti-Dsg3 antibodies into the media, which can neutralize the construct, similar to the physiological condition in PV patients. Despite this, we still observed similar effective killing between AK23 and F779 cells at a 10 nM dose, indicating that antibodies against Dsg3 have a minimal negative impact on the killing efficacy of the fusion protein. The dose dependency of killing can be evaluated, as this will have implications for animal studies and clinical translation. Similar experiments can be performed using mouse bone marrow derived macrophages-autoantibodies by standard ELISA Also, normal B lymphocyte counts can be demonstrated by CD19 and CD20 at the time of PBMC collection.

### Animal Studies.

Treatments: To test in vivo efficacy of the Dsg3-Fc treatment, fusion protein can be intravenously injected at 0, 2 or 5 days after AK23 injection (model 1). These time points can help us identify the relationship between disease burden and the effectiveness of the treatment, that is, to determine whether the treatments are preventative and/or can they be able to rescue severe disease phenotype. The treatment proteins can be used at varying concentrations (5 mg/kg-15 mg/kg 46,47 in 100 μl final volume via tail-vein IV injection) to find the optimum condition. The range can be determined based upon the antibody concentrations used for B cell depletion in autoimmune mouse models. For the active disease model (model 2), the treatments will be performed at Day 0, 14, or 21 post adoptive cell transfer in Rag2−/− mice, as previous studies have reported circulating anti-Dsg3 IgG was in the sera of recipient Rag2−/− mice as early as day 4 followed by a rapid increase and a plateau around day 21. Non-invasive readouts, such as anti-Dsg3 ELISA, weight loss, and skin lesions/erosions can be performed at various time points (Day 4, 10, 17, 25, and 35 post treatment) (n=10 mice for treatment and control groups). Trend obtained from these data can be used to establish the peak in control mice that did not receive the treatment. Studies have established peak to be between Day 10 and 20. Post-peak time point, skin biopsies can be obtained for immunofluorescence and histological staining of mucosal barriers. In both model 1 and 2, soluble anti-Dsg3 antibodies may partially neutralize the killing capability of our constructs. If so, we will use different amounts of the constructs to find the optimum dose (highest killing with lowest off-target rate). Controls: For negative controls, Dsg1-Fc and Dsg3-mutated Fc (with no Fc functionality), will be used at the same concentration as the treatment proteins. Furthermore, and as a positive control, we will explore using anti-CD20 IgG (clone SA271G2) at concentration 250 μg per animal, as it has been shown to effectively deplete B cells in mice 48 (which can be more effective in model 2, as AK23 hybridoma cells can or cannot be depleted completely with the anti-CD20 antibody). Readout: The readout for model 1 experiments will be bioluminescence imaging (IVIS) of AK23 hybridoma cells (as they are luciferase positive) that allows sensitive longitudinal measurements for the AK23 cells. Furthermore, for both models 1 and 2, serum anti-Dsg3 titers measured at days 5 and 14 post-initiation of treatments, blinded investigation of skin erosions, immunofluorescence of mucosa samples to detect IgG deposition, and histological assessment of mucosal barriers can be performed. B cell counts can be performed as well, to ensure they remain within normal levels during the treatment. Without wishing to be bound by theory, the treatments result in clinical and histological resolution of the disease.

### Additional Studies.

In vitro studies: The protein engineering and expression, cloning, and synthesis of the sortase peptides are routine in the PI's laboratory, and without wishing to be bound by theory, there is not difficulty in the production and scaling up of the protein expression and peptide synthesis. However, if the protein expression yield is low for a particular construct, we can explore different routes to address the issue: 1) re-optimize the codons used for the expression, 2) use a different signal peptide sequence, 3) use other systems for protein expression instead of Expi293F cells, such as CHO cells or insect cells. Without wishing to be bound by theory, the soluble polyclonal anti-Dsg3 antibody to partially, but not completely, neutralize the effect of our constructs. We can thus assess killing abilities of our constructs on F779, PVB28 and AK23 cells in the presence of polyclonal PV serum IgG. Further, we do not see this as a limitation in clinical practice, as the dosing of these constructs can be patient-dependent and based on the level of circulating anti-Dsg3 titer, similar to, but not directly analogous to, drugs like omalizumab (an IgG1k anti-human IgE, used based on the IgE level). Additionally, in real-world setting, our therapeutic can be used concurrently with other drugs that lower anti-Dsg3 titer. Further, monoclonal and polyclonal anti-Dsg3 IgG was found not to have a significant effect on the killing abilities of CAAR T cells specific for Dsg3-autoantibody producing B cells 16. To further increase the efficacy of this fusion protein, we have designed and successfully expressed Dsg3-Fc fused with IL-2, which (without wishing to be bound by theory) can engage more cells and enhance the killing. We can explore other cytokines (e.g., IL-15) as well. However, these approaches can or cannot have some side-effect. For negative control experiments, in addition to cells lacking expression of anti-Dsg3 antibodies, and as an alternative, we have designed and made a plasmid for synthesis of Dsg1-fusion that we can now express and use as well. In vivo studies: long term monitoring and any potential side-effects can be performed as explained above. We can carefully the dose-dependency of the treatment and how it correlates with the presence of anti-Dsg3 autoantibody titers (via ELISA). With the wide-spread use of biologics, anti-drug antibodies have been reported for multiple agents ranging from anti-TNFαs to rituximab. Major immune adverse events, such as immune-complex deposition, have been theorized, but have not actualized in clinical practice. In cases where the biologic directly binds to the high circulating antibodies, such as in the case of omalizumab, dosing patients with high IgE level did not result in immune adverse events. Similarly, without wishing to be bound by theory, the presence of anti-Dsg3 IgG in serum will not result in any major immune adverse events, but this will be monitored.

### Examining the Ability of Dsg3-Anti-CD3 Fusion to Mediate Killing of Anti-Dsg3 B Cells.

Synthesis of Dsg3 fusion to anti-CD3e scFv. The N-to-C Dsg3-anti-CD3e fusion expression plasmid was designed to contain cDNA for the hDsg3-EC1-3 and the OKT3 anti-CD3e scFv. OKT3 clone had been used in the clinic as the anti-CD3 portion of the bi-specific T cell engagers; CD3e is found on all mature T lymphocytes. The construct was designed as such to have the Dsg3 on the N-terminus and anti-CD3e on the C-terminus, similar to other bi-specific T cell engagers; furthermore, this structure can ensure that the EC1-EC3 domains will be available for recognition by the Dsg3-reactive B-cells. A flexible (G4S)3 spacer was included between Dsg3 and scFv proteins, similar to other bi-specific T cell engagers. The protein was expressed using the Expi293F cells.

C-to-C fusion using click chemistry: Without wishing to be bound by theory, through using sortase, we can site-specifically install click handles at the C-termini of Dsg3 and anti-CD3e scFv proteins, which then can allow us to “click” them together using azide-alkyne click chemistry. This will result in the production of a C-to-C fusion, which is impossible to make through traditional genetic approaches. Such an approach is site-specific, thereby the product can retain its functionality. To establish the approach, we can synthesize an anti-CD19-clicked to-anti-CD3 scFv fusion, analogous to blinatumomab, as we can use it as a positive control for the killing assays. We used the same sequence of the Blinatumomab for both scFvs (which consists of the anti-CD3 OKT3 clone). We first bacterially expressed the anti-CD3e scFv, equipped with a sortase recognition tag (LPETG) at its C-terminus. The anti-CD19 scFv was made similarly. Next, Gly3-alkyne and Gly3-azide sortase substrates were synthesized. The anti-CD3e scFv was installed with the alkyne substrate, and the anti-CD19 scFv was installed with the azide substrate, both at their C-termini via sortase. The click-functionalized scFvs were purified via FPLC and subsequently clicked together via the copper-mediated azide-alkyne cycloaddition chemistry (FIG. 8 panel A). The product was purified via FPLC and characterized using SDS-PAGE and flow cytometry analyses, which confirmed that the clicked C-to-C fusion stains both Nalm6 cells and human T cells with a strong affinity. We will use the approach to make C-to-C fusion of Dsg3-anti-CD3e as well. 2. Sortase-mediated synthesis of the N-to-C fusion: The click chemistry approach is an alternative to the traditional genetic fusion when and if the expression of a genetically fused heterodimer protein proves to be difficult. However, to provide a second alternative, and to make a more “natural” fusion, without wishing to be bound by theory, through sortase, we can make N-to-C fusion of Dsg3-anti-CD3e, without any prior modification on either of the proteins, as follows: Taking advantage of the sortase reaction reversibility, (and without wishing to be bound by theory) a triglycine molecule (GGG), can be used to catalyze the formation of a C-to-N protein fusion: protein A is engineered to have an “LPETG” motif at the C-terminus, and protein B will be engineered to have an “LPETGGG” at the N-terminus. The addition of sortase and the triglycine molecule can remove the “LPET” part from the protein B and convert it into “Gly3-B”. The Gly3-B protein, produced in situ, can then react with protein A to form the fusion product, “A-LPET-GGG-B” with no click chemistry involved (FIG. 8 panel B). The reaction can be done in a dialysis cassette allowing for the excess GGG catalyst to leave the reaction mixture, which would further move the reaction toward the formation of the fusion product. To establish this approach, we made such an “anti-CD19-anti-CD3 scFv fusion”. The anti-CD19 scFv has “LPETG” sortase tag at its C-terminus. We engineered the anti-CD3 scFv protein, to have the following sequence: “MHis6-LPETGGG-anti-CD3 scFv” (proteins always start with an M, as the methionine codon (ATG) serves as the start codon; His6 was used to facilitate purification). Next, we mixed the anti-CD19 and anti-CD3 scFvs, Gly3, and sortase and let the reaction proceed at 4° C. overnight. The product formation was confirmed via SDS-PAGE analysis and the fusion protein was purified via FPLC size-exclusion chromatography (FIG. 8 panel C); the fusion was used to stain Nalm6 cells (CD19+) and Jurkat cells (CD3+). The flow cytometry results confirmed the fusion stains the cells with high affinity (FIG. 8 panel D). This strategy is an unpublished approach, that can have wide-spread applications in the community to make C-to-N protein fusions that are difficult to express as genetic fusions. No prior modification on either of the proteins is required, except the introduction of sortase tags in the cDNA plasmids. This approach to make Dsg3-anti-CD3e fusion.

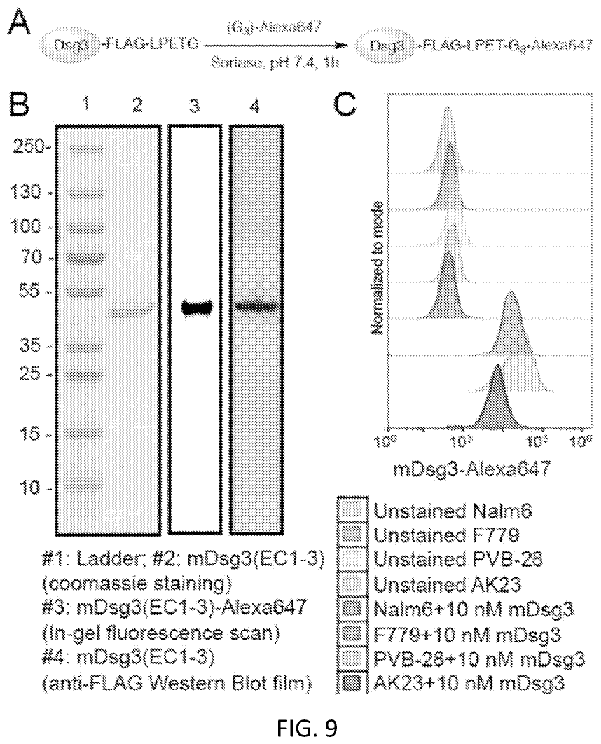

Mouse and human Dsg3-EC1-3 were made and characterized similarly (FIG. 9 panels A-B). We next used sortase to install Alexa647 at their C-termini (FIG. 9 panel A). To characterize the product, we stained anti-Dsg3 cells (F779, and AK23 cells). The Alexa647-labeled mDsg3-EC1-3 could stain the cells with high affinity and specificity (FIG. 9 panel C). The Nalm6 cells, used as control, did not show any staining. We will make and fully characterize Dsg3-anti-CD3 fusion (mouse and human) proteins following both the click and the direct sort-tagging approaches. We have made and characterized an anti-mouse CD3e scFv as well (clone 2C1) 61.

Measuring the killing efficacy of the constructs. Positive anti-Dsg3 cell lines (AK23, F779 and PVB-28) and negative cell line (Nalm6 and an irrelevant hybridoma) will be co-cultured with fresh human CD8+ cytotoxic T cells (E: T ratio of 1:1, 5:1, and 10:1). The chimeric Dsg3-anti-CD3e fusion protein will be incubated at 37° C. in a standard cell incubator. The fusion protein will be used at varying concentrations to find the optimum condition (10 μg/mL to 1 ng/mL). As a second control, cells will be incubated with medium alone, expecting no cell death difference between the anti-Dsg3 cells and control cells. Standard live-dead analysis of cells will be performed using flow cytometric analysis. Production of IFN-γ, as an indicator of T cell activation, will be assessed using ELISpot assay. Delineating the efficacy of the approach on patients PBMCs in in vitro assays: Similar to aim 1, hDsg3-anti-hCD3e fusion protein can be added to human PBMC culture obtained from PV patients; healthy PBMCs can be used as control. No additional T cells will be added to the mixture, as (without wishing to be bound by theory) the patients' own T cells to be directed and activated to kill the targeted anti-Dsg3 B cells. Varying concentrations of the fusion can be used to find the optimum condition (10 μg/mL to 1 ng/ml). The killing efficacy can be analyzed, 4 h, 12 h and 24 h after addition of the fusion (n=10 for each experiment); the analysis can be performed similar to the Fc approach.

Animal studies using Dsg3-anti-CD3e approach. Recombinant protein can be intravenously injected at 0, 2 or 5 days after AK23 cell injection (model 1) at varying concentrations (0.5 mg/kg-1 mg/kg), similar to those used in oncological treatment settings in 100 μl final volume to find the optimum condition. For the active disease model (model 2), recombinant protein will be injected at Day 0, 14, or 21 post adoptive cell transfer in Rag2−/− mice (as previously described). As negative control Dsg1-anti CD3e complex can be used at the same concentration as the treatment protein. For readout (as described in Aim 1), along with luciferase (for model 1), immunofluorescence and histological imaging, and anti-Dsg3 ELISA titers, cells can be isolated from the skin biopsies of mice with and without treatment. Normalized count of CD3+ cells will be analyzed through flow cytometry analysis at Day 5, 14 and 20 post treatment. There can be an increase in T cell counts in mice injected with Dsg3-anti-CD3e treatment confirming that the recombinant protein can recruit T cells. Long term monitoring and any potential side-effects will be performed as explained herein.

### Additional Studies.

In vitro studies: The sortase reaction can be monitored via SDS-PAGE and/or mass spectrometry. Typically, once we obtain >90% of the protein labeled with the sortase substrate, the product can be purified via size-exclusion chromatography. The azide-alkyne cycloaddition is a well characterized reaction extensively used for bioconjugation of proteins, with near complete yield. Final fusion constructs can be further characterized by mass spectroscopy, SDS-PAGE, flow cytometry and immunoblot analysis. The synthesis of the various modified peptides is routine in the laboratory, and (without wishing to be bound by theory) any difficulty in the production and scaling up of the synthesis of the modified peptides. However, some proteins can become less soluble upon addition of a hydrophobic substrate. If that becomes an issue, we will include a small polyethylene glycol (PEG) spacer, which is very hydrophilic, between the Gly3 and the alkyne functionality (e.g., Gly3-PEG5-propargyl alkyne). Furthermore, if the alkyne-azide click reaction does not result in high product yield for a given construct, we can use alternative click handles such as the more hydrophilic aldehyde and aminooxy click handles, which forms stable oxime product, and we have extensive experience with this. We have included a small PEG3 spacer between the Gly3 and the alkyne or azide functionalities in the sortase substrate molecules. Without wishing to be bound by theory, the PEG spacer can lead to a higher stabilization of the construct; this PEG spacer size can be changed if a given fusion has low stability or has low solubility. Furthermore, we can analyze whether the size of the spacer affects the killing abilities of the constructs. We have shown that our mouse and human Dsg3-EC1-3 constructs, bacterially expressed and then refolded, binds with great affinity (low nanomolar) to all the anti-Dsg3 cells and antibodies that we have in hand. Furthermore, glycosylation of Dsg3 was shown not to play a significant role in its binding to patient anti-Dsg3 antibodies. Therefore, we will continue to use our established approach to make Dsg3 constructs. We will continue our optimization effort to increase the yield of the genetic fusion expression through alternatives that were already discussed in aim 1 (such as codon optimization, different signal sequence, and different expression systems), as it can be more relevant when considering clinical translation. For the Fc constructs discussed in aim 1, all proteins were, and will be, expressed in mammalian Expi293F cells, as glycosylation is critical for the Fc-functionality, and we already have good expression yields for the Fc constructs (˜1 mg per 100 ml of Expi293 cells). In vivo studies: without wishing to be bound by theory a side-effect for the anti-CD3 treatment can be the engagement of CD4 T cells, for example T follicular helper cells (Tfh), with autoreactive B cells, which can result in their expansion and thus exacerbation of the disease. Without wishing to be bound by theory, this will not happen, as the bi-specific T cell engagers used in the clinic are effective in recruiting CD8 cytotoxic T cells to kill the target cells. Without wishing to be bound by theory, the frequency of CD8 T cell recruitment can be higher than the frequency of recruited Tfh cells; this is because most, if not all, of CD8 T cells, with any TCR, can be recruited to kill the autoreactive Dsg3 cells, whereas engagement of CD4 T cells (expressing unrelated TCR) with the autoreactive B cells is not expected to result in B cell expansion; perhaps engagement of only a few CD4 T cells, with reactivities against Dsg3-derived peptide-MHC complexes, can result in an expansion of autoreactive B cells. Furthermore, there is no report of development of autoimmunity in patients that received bi-specific T cell engagers (e.g., blinatumomab) and given the incidence of different types of B-cell mediated autoimmune disease is not insignificant, this observation suggests the occurrence of such event is unlikely. Nevertheless, we will monitor this issue. The active immune model of PV (model 2) can be the ideal model for studying this issue, which will be carefully assessed by monitoring the anti-Dsg3 titer, the number of Dsg3 autoreactive B cells, and the number and phenotype of CD4 T cells, especially Tfh cells. Similar to the Fc-mediated approach, the effect of anti-Dsg3 antibodies on this therapeutic is unlikely to have clinical significance; Additionally, in real-world setting, our therapeutic can be used concurrently with other drugs that lower anti-Dsg3 titer. Nevertheless, this issue can be monitored and assessed.

### Investigation of the Ability of Alpha-Emitting Labeled Dsg3 to Kill Anti-Dsg3 B Cells.

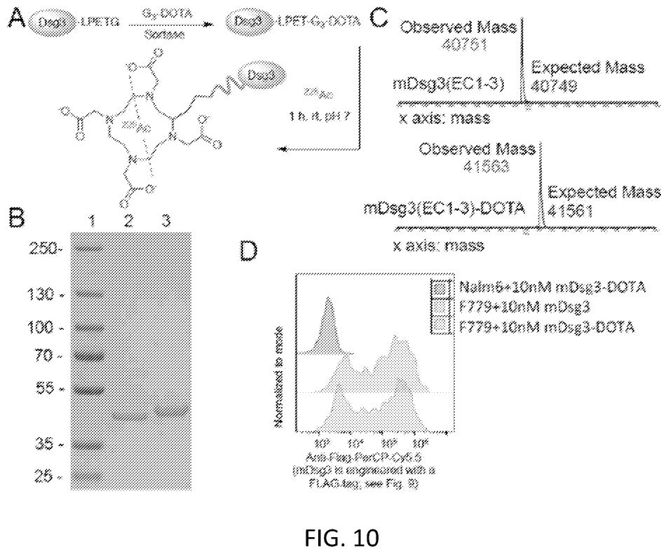

Radiolabeling of desmoglein-3 protein. Among the available isotopes, Actinium-225 (²²⁵Ac) (t1/2=9.9 d) has been used in the clinic 25,71. Labeling of antibodies with ²²⁵Ac can be performed via the well-characterized DOTA chelator molecule. We have synthesized a Gly3-DOTA sortase substrate and site-specifically modified mDsg3-EC1-3 protein with the substrate, characterized the product and confirmed the protein remained functional with high binding affinity to the anti-Dsg3 cells (FIG. 10). Next, the DOTA-labeled Dsg3 will be installed with a ²²⁵Ac radioisotope. Formation of product and radiochemistry yield will be determined using radio-HPLC analysis. Stability of the purified ²²⁵Ac-Dsg3 constructs will be assessed by incubation in PBS and in human serum at 37° C. To ensure radiolabeling will not affect functionality of the desmoglein protein, positive anti-Dsg3 cells and negative cells will be incubated with ²²⁵Ac-Dsg3 for 30 min. Cells will be washed, 3 times, and radioactivity will be measured. We expect the anti-Dsg3 cells to be radioactive, while the negative cells can or cannot have any radioactivity. This can confirm that radiolabeling procedure had not compromised Dsg3 protein's binding capacity.

Measuring the killing efficacy of the radiolabeled constructs using the established anti-Dsg3 cells. Positive anti-Dsg3 hybridoma cells and negative cells, will be cultured in round bottomed 48-well plates. The radiolabeled ²²⁵Ac-Dsg3 protein will be added to the cells at 37° C. The radiolabeled recombinant construct will be used at varying concentrations to find the optimum condition (10 μCi/mL to 1 nCi/mL). As control, cells will be incubated with same amount of ²²⁵Ac-DOTA alone (not conjugated to Dsg3), expecting no anti-Dsg3 cells to be killed. Incubation with radiolabeled Dsg3 or the control isotope, will be done for 20 min, followed by washing out (3×) any unbound reagent/isotope to prevent non-specific cell death from irradiation. Analyses will be performed 4 h, 16 h, 24 h and 48 h post-incubation of the cells and the radiolabeled Dsg3, via flow cytometry, and standard live-dead analyses.

Evaluating the efficacy of the ²²⁵Ac-radiolabeled-Dsg3 construct on PV patient PBMCs in in vitro assays. Similar to the aims 1 and 2, the ²²⁵Ac-radiolabeled-Dsg3 construct will be added to the human PBMC cells (both healthy and patient samples) and the mixture will be incubated at 37° C. in a standard cell incubator. Varying concentrations of the construct will be used to find the optimum condition (10 mCi/mL to 1 nCi/mL). As control, cells will be incubated with same amount of ²²⁵Ac-DOTA alone. Incubation with radiolabeled Dsg3 or the control isotope, will be performed for 20 min, followed by washing out (3×) any unbound reagent/isotope to prevent non-specific cell death from irradiation. Analyses will be performed at 4, 16, 24 and 48 h post-incubation with radiolabeled Dsg3, via an ELISpot assay for antigen-specific and total IgG B-cell depletion. We will measure the percentage decrease of the autoreactive anti-Dsg3 IgGs and total IgGs level. Furthermore, analysis will be performed via flow cytometry, and standard live-dead analyses on the nonspecific cells, including B and T cells, which will act as an internal standard for the assay. Controls, inclusion and exclusion criteria will be same as mentioned in aims 1 and 2. Expected results will confirm targeted killing of Dsg3 autoreactive B cells.

Animal studies using Radiotherapy approach. Radiolabeled recombinant protein will be intravenously injected at 0, 2 or 5 days after AK23 cell injection (model 1), with the ²²⁵Ac-labeled Dsg3 constructs (˜30 kBq activity and 50 μg Dsg3 per animal⁷²). We will use ²²⁵Ac-DOTA (the chelator molecule) as control (same amount of activity (˜30 kBq), ˜3.0 μg DOTA per animal). For readout, along with luciferase, immunofluorescence and histological imaging, and anti-Dsg3 ELISA titers, cells will be isolated from the skin biopsies of mice with and without treatment, and activities will be measured using a gamma counter. Similar experiments will be performed for the active immune model², following experimental strategy as explained in aim 1. Ex vivo biodistribution studies will be performed as we have described^{29,30}. The animals will be euthanized at 2, 6, 24, and 48 h post-injection. Next, their major organs and tissues will be isolated and weighed, and their radioactivity will be measured along with that of the injection standards (²²⁵Ac-DOTA as controls). The results will be analyzed and reported as a percentage of the injected dose per gram of tissue (% ID/g). Measurement of circulatory half-life will be performed through collecting drops of blood at multiple time points, followed by their activity measurement.

### Additional Studies.

In vitro studies: We have extensive experience in radiolabeling proteins. Radiolabeling can affect binding affinity of autoreactive antibodies on Dsg3 proteins. This can be due to the high positive charge of actinium cation (3+). If the radiolabeling affects the binding capability of the Dsg3 protein, we will synthesize a sortase substrate with a PEG spacer (for example Gly3-PEG5-DOTA). This should allow proper distance between the Ac cation (3+) and the Dsg3 protein. Syntheses of the peptides are routine in our laboratory. We chose to use ²²⁵-Ac isotope, not only because it has been used in the clinic, but because it has unique benefits over other existing isotopes, such as limited range in tissue, good linear energy transfer, relatively short half-life (˜10 days), and multiple (4 net) alpha particles emitted with each decay. If using ²²⁵-Actinium revealed to have other unexpected issues, we can use ²¹³-Bismuth, also used in the clinical setting, as an alternative. In vivo studies: The issue of radiotherapeutics is renal toxicity. We will monitor for renal toxicity and use approaches such as attachment of polyethylene glycol (PEG), a biologically inert and hydrophilic polymer to decrease the kidney uptake. We have shown that PEGylation decreases kidney uptake of radiolabeled antibody fragments in preclinical studies (by >10 times). Furthermore, PEGylation has been shown to be safe in the clinic, as there are several approved PEGylated protein therapeutics. We will use different size PEG (5, 10 and 20 kDa) to evaluate the effect of PEGylation on the kidney clearance. Alternatively, we can co-inject mice with 150 mg/kg Gelofusine, which have been shown to reduce kidney uptake and renal toxicities. We can study the pharmacokinetics and tissue biodistribution of the constructs in vivo, through labeling the constructs with a PET isotope (⁶⁴Cu and ⁸⁹Zr that have a t_{1/2} of ˜12.7 h and ˜3.3 days, respectively), with which we have extensive experience. Whole-body PET images will not only reveal tissue biodistribution and kinetics, but it will also reveal whether any of the constructs can potentially be sequestered in any organs, which, in turn, may result in organ damage. The PET imaging experiment will, therefore, reveal what the potential side-effects can be, if any. We do have extensive experience with PET imaging. Ex vivo autoradiography will be performed as well, to quantitatively measure tissue biodistribution with a very high sensitivity. Similar to the previous two approaches, the effect of anti-Dsg3 autoantibodies on this therapeutic will be carefully assessed. The half-life of ²²⁵Ac is relatively short (˜10 d). However, if increased circulatory half-life of ²²⁵Ac-Dsg3 occurs due to complexation with autoantibodies and recycling through FcRn thereby potentiating side-effects on cells that may take up the complex, the clinically used ²¹³-Bi isotope, which has a very short half-life (˜45 min), can be used. This should address the side-effect issue arising from the complex formation between radiolabeled-Dsg3 and autoantibodies. Considering clinical translation, this approach can be an option for patients who fail existing treatments, with potential future applications to be expanded to other autoimmune blistering diseases, for example, in paraneoplastic or immunotherapy induced autoimmune blistering diseases when the use of immunosuppression is associated with worse outcome⁸³. Additionally, similar to Fc-mediated and bi-specific T cell engager approaches, in the real-world setting, the radiotherapy approach may be used concurrently with other drugs that lower anti-Dsg3 titer and/or saturate FcRns, such as IVIG.

Additional in vivo studies: The passive transfer model has been established in Rag2−/− mice 32,34. We can establish it in immunocompetent WT mice and optimize the number of hybridoma cells needed for the same. Alternatively, the AK23 passive transfer model in Rag2−/−, will receive WT C57BL/6 splenocytes, which can resemble an immunocompetent host. Furthermore, for proposed treatments, we will perform toxicology screens by using primary human cells and high-throughput membrane proteome arrays to identify any off-target cytotoxic interactions for each of the developed treatments. Without wishing to be bound by theory, the Dsg3-containing constructs will not incorporate themselves into desmosomes in the skin, as it requires the full structure to be on the cell-surface including the transmembrane domain for a potentially long-term stability. Furthermore, extensive studies on Dsg3-CAAR T cells in animal models did not show any accumulation of the Dsg3-CAAR in the animal skin and, similarly, when human skin was transplanted into mice skin, the hDsg3-CAAR T cells were not detected in the transplanted skin, indicating the Dsg3 constructs are not expected to incorporate themselves into the desmosome. This will be studied through histology analyses. We can determine whether the existing anti-Dsg3 antibodies bind to the reagent, affecting biodistribution, or resulting in interaction with cells expressing the Dsg3-autoantibodies Fc receptors. This will be studied in both animal models, and through extensive histology, PET and flow cytometry analyses. Without wishing to be bound by theory, this will not be a major issue as the biologics we are developing will have short circulatory half-lives and, unlike adoptive cell therapy such as CAAR T cell treatments, will not stay in the recipient (mice or eventually patients); the half-lives of the developed therapeutics can be within only a few hours for the radiotherapeutics and the anti-CD3 fusions, and several hours to potentially a few days or even few weeks for the Dsg3-Fc constructs (which can be recycled via FcRn). In order to test if our treatment had any adverse effects on other immune cell compartment, PBMCs will be collected from mice that received our proposed therapy and percentages of Th1, Th2, Th17, Tregs, and CD8 T cells will be analyzed by flow cytometry analysis. Age and sex matched unmanipulated WT B6 mice will be used as control. Without wishing to be bound by theory, there will be no difference in the percentages of immune cell population suggesting no adverse long-term effect of the treatments.

The approaches herein to target autoreactive B cells as well as our strategies to develop these therapeutics (site-specific installation of the radioisotope, synthesis of C-to-C fusions and N-to-C sortase mediated fusions) are unique. Our strategy of making Dsg3-Fc is safe and can replace the use of Rituximab. The other two approaches provide alternative options for patients who fail to respond to available treatments. Therefore, the compositions and methods described herein can transform the treatment of not only PV, but open an avenue for developing treatments for several autoantibody/immune-complex mediated diseases to known antigenic targets, ranging from other autoimmune blistering diseases (e.g., bullous pemphigoid, mucous membrane pemphigoid, epidermolysis bullosa acquisita, linear IgA bullous dermatosis), subtypes of lupus, scleroderma, Goodpasture's disease, Graves' disease, to immune-mediated vasculitis. We, therefore, believe the proposed therapeutics can be considered a ground-breaking addition to the therapeutic toolbox for autoimmune diseases.

### Example 3

Pemphigus vulgaris is a devastating B-cell mediated autoimmune disease. Autoantibodies targeting desmoglein 3 (Dsg3), a desmosomal protein critical for intercellular adhesion of skin and mucous membrane, is responsible for the disease phenotype. Standard treatments include immunosuppressive drugs that systemically suppress the immune system. We have developed a targeted, immuno-therapeutic approach that specifically and precisely targets and depletes only autoreactive B cells recognizing Dsg3. Our in vitro and in vivo experiments indicate the approach results in efficient and specific depletion of the autoreactive B cells. We can determine the efficacy and specificity of the approach using peripheral blood mononuclear cells (PBMCs) obtained from PV patients in different phases of the disease and perform additional in vivo studies using mouse models of passive and active disease. We will perform studies in dogs, which develop the disease with similar pathogenesis and phenotype to humans. Without wishing to be bound by theory, the methods can be translated into a clinical setting. The technology developed herein can have wider applications to all autoantibody-driven diseases by targeting autoreactive cells without causing global immunosuppression.

### Background

Pemphigus vulgaris (PV) is a fatal autoimmune blistering disease, characterized by painful blistering and erosions of the skin and mucosa limiting activities of daily living (e.g., eating, drinking) with impact on one's overall well-being and high morbidity and mortality¹⁻³. PV is caused by production of autoantibodies against desmoglein 3 (Dsg3), with or without antibodies against Dsg1, resulting in epithelial detachment (acantholysis)^{2,4-6}. There is a therapeutic need in pemphigus treatment. There is no cure for PV; treatment strategies rely heavily on systemic steroids and immunosuppressive drugs⁷⁻⁹ with a multitude of systemic side-effects. While anti-CD20 antibodies (e.g., rituximab)³ is a first line option for moderate-to-severe disease, it is associated with off-target immunosuppressive effects including obtunding humoral response to vaccines with current, real lifetime implications, and increased risk of infection albeit lower than traditional immunosuppressive agents^{10,11} and a high risk of relapse after treatment¹⁰⁻¹². Neonatal Fc receptor and Bruton's Tyrosine Kinase inhibitors, currently in clinical trials, cannot circumvent the off-target immunosuppressive effects. Early-stage cell-based therapies such as autologous polyclonal regulatory T cells 13 and chimeric autoantigen receptor (CAAR) T cells¹⁴, if proven effective, cannot be globally immunosuppressive. However, cell-based therapies must be personalized, have specific requirements for manufacturing, storage and transport, which cannot be feasible in resource-poor areas. Relapse in PV is typically due to expansion of the same autoreactive cells, not depleted during initial treatment²; without wishing to be bound by theory, complete depletion of autoreactive B cells will offer a cure. Herein, we describe a treatment that is targeted to specific autoreactive B cells by using the autoantigen itself (Dsg3) and commonly used Fc-mediated killing. This method will selectively target autoreactive, disease-causing cells, without affecting global immune function.

Pemphigus vulgaris affects a young, healthy group of patients (i.e. young/middle aged-adults, average 40-60 years of age) compared to other autoimmune blistering diseases. Current existing treatment strategies is limited to immunosuppressive medications—for example, systemic steroid, mycophenolate mofetil, azathioprine, and rituximab, with risk of infection, higher risk of malignancy in the long run and obtunding response to new vaccination with current, real-life implications. The disease can be brought into remission with intense period of immunosuppression, but the risk of recurrence is as high as 80% 10. Study shows that patients with pemphigus have similar risk of post-traumatic stress as patients after a cancer diagnosis, both conditions are life-threatening with frequent recurrences 16. Patients with this disease group need a targeted treatment that eliminate the autoreactive cells without impacting their overall health and immune function. Clinicians treating these have limited repertoire of treatments to offer, all with long list of toxicities, short and long-term adverse health effects, including life-threatening infections. From the insurance payer perspective, a targeted therapy can lower the overall global healthcare cost by lowering risk of infection in the immediate term, long-term risk of malignancy, decrease the number of visits, ancillary treatments and hospitalizations. This targeted, non-immunosuppressive treatment can be attractive to patients, clinicians and insurance payers alike.

The clinical effect of the therapeutic is visible to the human eye and there is commercially available serum test for monitoring recurrence. The disease prevalence in the US is estimated to be 5.2 cases per 100,000 adults 15. Patients can have relapses throughout their life, requiring repeat treatments. There is a higher incidence in those of Jewish ancestry (˜4- and 10-fold higher than the average) 15.17. Existing treatments (i.e. prednisone, mycophenolate mofetil, azathioprine, rituximab) and up-and-coming treatment (e.g., neonatal Fc receptor antagonist, Burton's Tyrosine Kinase inhibitors) are immunosuppressive, or difficult to generalize to a global market (e.g., CAART). The method herein is based on the common shared autoantigen which can be manufactured without the need for personalization.

### Non-Limiting Exemplary Product/Solution

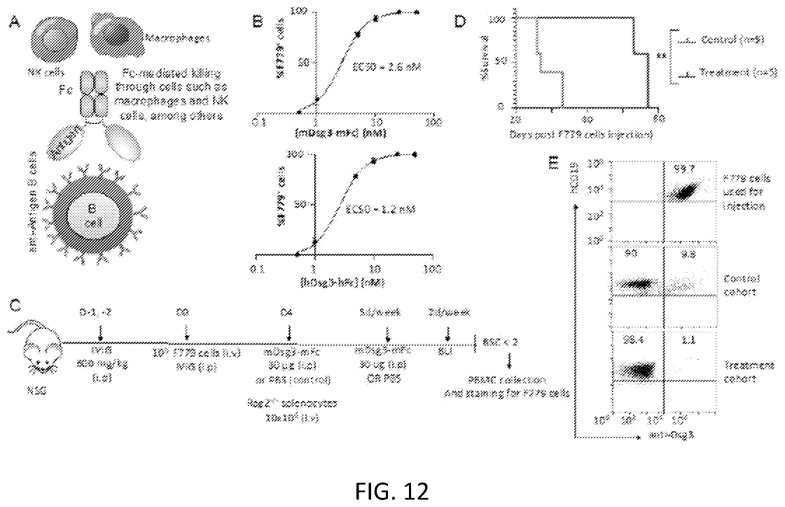

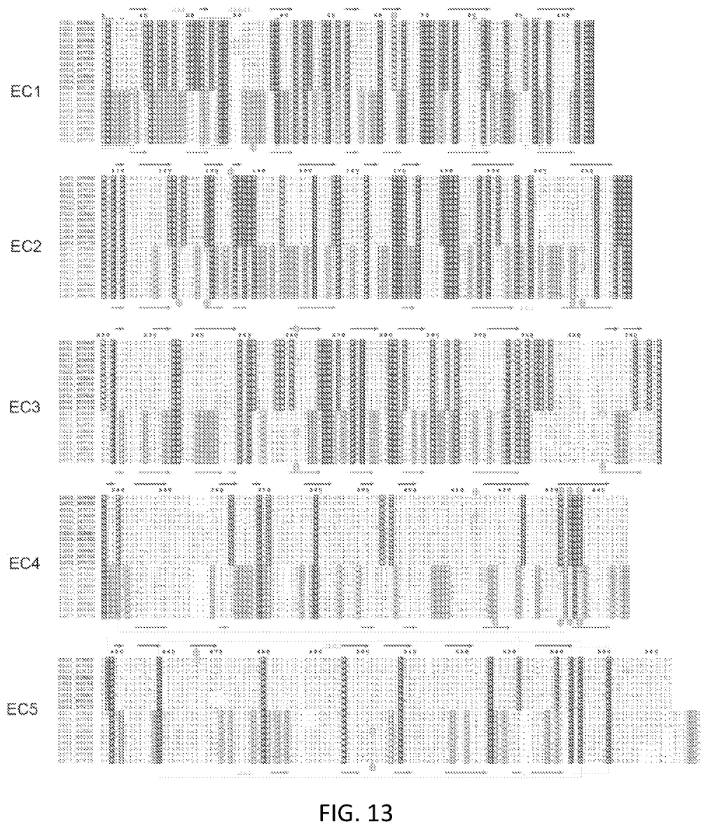

The treatment described herein can be used in an outpatient setting in a manner analogous to Rituximab (which can be given in an outpatient infusion center). This treatment will be used both in the initial stage of treatment and with relapses. The disease population will be patients with mucosal-dominant pemphigus vulgaris, with a clear goal to expand to other autoimmune blistering diseases in the future, such as pemphigus foliaceus (where the main autoantigen is Dsg1) in the next step. This treatment can replace current standard of treatment, Rituximab (anti-CD20), as it is selective and does not have off-target effect/global immunosuppression from complete depletion of B cell pool by Rituximab. The antigen-Fc (Dsg3-Fc) fusion protein will effectively and specifically kill Dsg3-recognizing cells. The Dsg3-portion of the construct will bind to the anti-Dsg3 BCR of autoreactive pathogenic B cells and the Fc domain will induce Fc-mediated killing of the Dsg3-recognizing cells by recruitment of phagocytic and natural killer (NK) cells (FIG. 12 panel A). We have engineered and made mouse Dsg3 and human Dsg3 constructs fused with the mouse IgG2a and the human IgG1 Fc domain, respectively, to achieve maximum Fc-mediated cellular cytotoxicity. We have shown that, in both in vitro and in vivo studies, the treatments are capable to target and mediate killing of the autoreactive B cells.

### Non-Limiting, Exemplary Data

We have made both mouse and human Fc-fusion proteins: mDsg3-mIgG2a (mFc) and hDsg3-hIgG1 (hFc). We used ectodomains 1-3 (EC1-3) of the Dsg3 protein as that can be an immunogenic and pathogenic portion (FIG. 13)¹⁸. Human IgG1 and mouse IgG2a were chosen for their high Fc-mediated killing activities¹⁹⁻²¹. The proteins were engineered to have a FLAG-tag, a sortase recognition motif22,23 and a His6-tag at the C-terminus. The FLAG, an 8 amino acid sequence (DYKDDDDK), can be used for Western Blot and flow cytometry analyses, and subsequently for analysis of the construct metabolic fate when used in vivo. The His6-tag was used for purification and the sortase tag was used to site-specifically introduce modifications, such as attachment of fluorophores. To characterize the functionality of the constructs, we used Alexa647-mDsg3-mFc to stain the anti-mDsg3 AK23 hybridoma²⁴ and anti-hDsg3 F779 cells¹⁴, a B lymphoma cell line (Nalm6) engineered with a patient-derived pathogenic anti-Dsg3 antibody. The cells were stained with great affinity and specificity (at low nM concentrations) (FIG. 12 panel B), suggesting that the patient-derived BCRs on the F779 cells cross-react with mouse Dsg3 protein. Nalm-6 cells, used as control, did not show any binding. Similar experiments were performed using the Alexa647-labeled hDsg3-hFc, which confirmed that the construct binds strongly to the F779 cells (FIG. 12 panel B) and stained the mouse AK23 cells too (similar to FIG. 12 panel B).

First, we confirmed that the Dsg3-Fc fusion, through its Fc (IgG2a), binds to the Fc-g receptors on mouse macrophages with low nM affinity; control Nalm6 cells remained unstained. To determine whether the fusion protein can result in Fc-mediated killing, F779 or AK23 target cells, co-cultured with mouse RAW264.7 macrophage cells, were treated with the mDsg3-mFc fusion protein. A 10 nM dose resulted in 65% and 60% killing of the F779 and AK23 cells, respectively, after 24 hours, with no killing of either Nalm6 or hybridoma control cells. At a 48-hour timepoint, we observed an impressive 94% killing of F779 cells with no change in the control Nalm-6 cells. AK23 hybridoma cells release anti-Dsg3 antibodies into the media, which can neutralize the construct, similar to the physiological condition in PV patients. Despite this, we still observed similar effective killing between AK23 and F779 cells at a 10 nM dose, indicating that antibodies against Dsg3 will have a minimal impact on the killing efficacy of the fusion protein.

Establishing the in vivo efficacy of Dsg3-Fc constructs. NSG (NOD-scid-gamma) mice were pretreated with 600 mg/kg IVIG (to block nonspecific killing via FcγR) for 2 days prior to tail vein intravenous (i.v.) injection of luciferase-positive F779 cells (1 million) (FIG. 12 panel C). On day 4 post F779 injections all mice were injected with 10×10⁶ Rag2−/− splenocytes, which has functional macrophages, i.v. followed by intraperitoneal (i.p.) injection of 30 μg of the fusion protein to the treatment group and PBS to the control group (n=5 for each cohort). mDsg3-mFc was injected 5 times a week to ensure enough drug is available in the circulation, for the duration of the study. The disease progression was monitored by bioluminescence imaging and mice body scoring. Upon BSC<2, PBMC was collected via submandibular bleeding and animals were euthanized. The result showed the control group had a mean survival of 26 days, whereas the treatment group had a survival of 57 days. (FIG. 12 panel D). The flow cytometric analyses of PBMC from the two groups revealed that the treatment group had near 100% antigen-loss (FIG. 12 panel E), indicating that the treatment was effective in mediating the depletion of antigen-positive F779 cells. Of note, the F779 cells are malignant and thus rapidly proliferate. This is not the case in the clinic where the pathogenic B cells have normal proliferation capacity. Furthermore, antigen-loss is not an issue in patients: in case the autoantigen is lost on a pathogenic B cell, then it would no longer be pathogenic. We can repeat the experiment with a lower dose of the treatment to determine the lower end of the dose needed for a full response.

### Non-Limiting, Exemplary Strategy

To further test the ability of Dsg3-Fc fusion proteins to mediate killing of anti-Dsg3 B cells in different mice models of the disease.

Passive Transfer Model. In this model, we can develop a passive monoclonal pemphigus mouse disease model by implanting an established pemphigus-inducing hybridoma cell line (AK23; already in hand). As established^{24,25}, we will inject ˜1 million AK23 hybridoma cells, which secrete anti-Dsg3 antibodies that cause the pemphigus phenotype into NSG mice^{24,25} Animals will be assigned in all experiments to treatment/control groups based on an urn randomization method. We will test for establishment of the disease by analyzing serum anti-Dsg3 titers (by standard ELISA on Day 3 and Day 10), and skin blisters (assessed by blinded investigators on Day 2, 5 and 10). Based on the ELISA data, time point for the tissue harvest will be determined to observe acantholysis (by H&E staining). The peak disease time is expected to be around Day 7²⁴; we expect the mice to exhibit hair loss²⁴, weight loss, and have blistering in hair follicles and oral mucosa along with AK23 autoantibodies produced by AK23 cells binding to epidermal keratinocytes with physiologic anti-Dsg3 IgG levels (300-400 RU/ml)²⁶.

Active Transfer Model. AK23 model has advantages such as easy read out, however, treatment can overcome the fast-proliferating hybridoma cells. Therefore, as a second model, we will use an active PV immune model that mimics the clinical phenotype of human disease, has lower frequency of autoreactive Dsg3-specific B cells that resembles what happens clinically, and finally has a high serum polyclonal autoantibody level. In this model, we can develop an active polyclonal pemphigus mouse disease model by first immunizing Dsg3−/− mice (JAX-002911) with recombinant extracellular domain of mouse Dsg3, followed by the transfer of splenocytes into RAG2−/− mice^{27,28} Transferred cells will contain T cells and B cells, which would include Dsg3-reactive cells. Similar to observations made in the literature²⁷⁻²⁹, we expect animals to have stunted growth and develop mucocutaneous erosions with suprabasal acantholysis and blisters. To assess the clinical index, using ELISA we will compare autoantibody levels to physiological levels in PV patients (300-400 RU/ml)²⁶. When the Dsg3 reactive antibodies equal to or exceed that level, which is expected to happen in 3-4 weeks^{26,30}, treatment will be initiated. Adoptive transfer of cells from Dsg3-immunized Dsg3+/− animals will be used as a control 29 (n=10 mice/group).

To test the ability of Antigen-Fc fusion proteins to mediate depletion of autoreactive B cells in dogs. Dogs, cats and horses develop pemphigus with similar pathogenesis to that of humans³¹⁻³⁷. Pemphigus is the most common autoimmune skin disease diagnosed in veterinary medicine, for which there is no cure, and the prognosis remains poor³¹⁻³⁷. The common autoantigens in canine pemphigus are, Dsg3 (pemphigus vulgaris) and Dsc1 (desmocollin-1; pemphigus foliaceus (PF))^{31,38-41}.

Since pemphigus in canine is developed because of autoreactivity against the desmosomal proteins and has a similar phenotype to humans, canine models can serve as a bridge between preclinical and human trials. To further assess the ability of our targeted treatment and to set the stage for clinical translation, we will treat dogs diagnosed with pemphigus using our pre-tested approach in mice. We will make a similar construct, using canine-Dsg3 and Dsg1 fused with canine IgG1 that is equivalent to human IgG1⁴². Production of such proteins is now straightforward in our lab and we anticipate no obstacles in making a large amount of these proteins. Improvement of lesions and autoantibody titers in circulation during the first three months of treatment will be carefully assessed.

Successful completion can set the stage for a clinical translation of the approach. The compositions and methods described herein can transform the treatment of not only pemphigus, but also open an avenue for developing treatments for several autoantibody/immune-complex mediated diseases to known antigenic targets, ranging from other autoimmune blistering diseases (e.g., pemphigus foliaceus, bullous pemphigoid, mucous membrane pemphigoid, epidermolysis bullosa acquisita, linear IgA bullous dermatosis), subtypes of lupus, psoriasis, scleroderma, Goodpasture's disease, Graves' disease, to immune-mediated vasculitis. As an alternative, we have made fusions of antigen-anti-CD3 antibody (Dsg3-fused to anti-CD3 scFv antibody) to engage T cells to the targeted cells, analogous to the bi-specific T cell engager therapeutics available in the clinic. While we are developing targeted therapy for pemphigus, the compositions and methods described herein can be to the therapeutic toolbox for autoimmune diseases.

### References Cited in This Example

1. Regezi J A, Sciubba J, Jordan R C. Oral pathology: clinical pathologic correlations. Elsevier Health Sciences; 2016.
2. Kasperkiewicz M, Ellebrecht C T, Takahashi H, Yamagami J, Zillikens D, Payne A S, Amagai M. Pemphigus. Nat Rev Dis Primers. 2017 May 11; 3:17026. PMCID: PMC5901732
3. Murrell D F, Peña S, Joly P, Marinovic B, Hashimoto T, Diaz L A, Sinha A A, Payne A S, Daneshpazhooh M, Eming R, Jonkman M F, Mimouni D, Borradori L, Kim S-C, Yamagami J, Lehman J S, Saleh M A, Culton D A, Czernik A, Zone J J, Fivenson D, Ujiie H, Wozniak K, Akman-Karakaş A, Bernard P, Korman N J, Caux F, Drenovska K, Prost-Squarcioni C, Vassileva S, Feldman R J, Cardones A R, Bauer J, Ioannides D, Jedlickova H, Palisson F, Patsatsi A, Uzun S, Yayli S, Zillikens D, Amagai M, Hertl M, Schmidt E, Aoki V, Grando S A, Shimizu H, Baum S, Cianchini G, Feliciani C, Iranzo P, Mascaró J M, Kowalewski C, Hall R, Groves R, Harman K E, Marinkovich M P, Maverakis E, Werth V P. Diagnosis and management of pemphigus: Recommendations of an international panel of experts. J Am Acad Dermatol. 2020 March; 82 (3): 575-585.e1. PMCID: PMC7313440
4. Mascaró J M, España A, Liu Z, Ding X, Swartz S J, Fairley J A, Diaz L A. Mechanisms of acantholysis in pemphigus vulgaris: role of IgG valence. Clin Immunol Immunopathol. 1997 October; 85 (1): 90-96. PMID: 9325074
5. Anhalt G J, Labib R S, Voorhees J J, Beals T F, Diaz L A. Induction of pemphigus in neonatal mice by passive transfer of IgG from patients with the disease. N Engl J Med. 1982 May 20; 306 (20): 1189-1196. PMID: 7040962
6. Furue M, Kadono T. Pemphigus, a pathomechanism of acantholysis. Australas J Dermatol. 2017 August; 58 (3): 171-173. PMID: 28211055
7 Ahmed A R, Moy R. Death in pemphigus. J Am Acad Dermatol. 1982 August; 7 (2): 221-228. PMID: 7130483
8. Tavakolpour S, Mahmoudi H, Balighi K, Abedini R, Daneshpazhooh M. Sixteen-year history of rituximab therapy for 1085 pemphigus vulgaris patients: A systematic review. Int Immunopharmacol. 2018 January; 54:131-138. PMID: 29132070
9. Joly P, Maho-Vaillant M, Prost-Squarcioni C, Hebert V, Houivet E, Calbo S, Caillot F, Golinski M L, Labeille B, Picard-Dahan C, Paul C, Richard M-A, Bouaziz J D, Duvert-Lehembre S, Bernard P, Caux F, Alexandre M, Ingen-Housz-Oro S, Vabres P, Delaporte E, Quereux G, Dupuy A, Debarbieux S, Avenel-Audran M, D'Incan M, Bedane C, Bénéton N, Jullien D, Dupin N, Misery L, Machet L, Beylot-Barry M, Dereure O, Sassolas B, Vermeulin T, Benichou J, Musette P, French study group on autoimmune bullous skin diseases. First-line rituximab combined with short-term prednisone versus prednisone alone for the treatment of pemphigus (Ritux 3): a prospective, multicentre, parallel-group, open-label randomised trial. Lancet. 2017 May 20; 389 (10083): 2031-2040. PMID: 28342637
10. Kushner C J, Wang S, Tovanabutra N, Tsai D E, Werth V P, Payne A S. Factors Associated With Complete Remission After Rituximab Therapy for Pemphigus. JAMA Dermatol. 2019 Dec. 1; 155 (12): 1404.
11. Wang H-H, Liu C-W, Li Y-C, Huang Y-C. Efficacy of rituximab for pemphigus: a systematic review and meta-analysis of different regimens. Acta Derm Venereol. 2015 November; 95 (8): 928-932. PMID: 25881672
12. Mahmoudi H, Balighi K, Tavakolpour S, Daneshpazhooh M. Unexpected worsening of pemphigus vulgaris after rituximab: A report of three cases. Int Immunopharmacol. 2019 June; 71:40-42. PMID: 30877872
13. Esensten J H, Muller Y D, Bluestone J A, Tang Q. Regulatory T-cell therapy for autoimmune and autoinflammatory diseases: The next frontier. J Allergy Clin Immunol. 2018 December; 142 (6): 1710-1718. PMID: 30367909
14. Ellebrecht C T, Bhoj V G, Nace A, Choi E J, Mao X, Cho M J, Di Zenzo G, Lanzavecchia A, Seykora J T, Cotsarelis G, Milone M C, Payne A S. Reengineering chimeric antigen receptor T cells for targeted therapy of autoimmune disease. Science. 2016 Jul. 8; 353 (6295): 179-184. PMCID: PMC5343513
15. Wertenteil S, Garg A, Strunk A, Alloo A. Prevalence Estimates for Pemphigus in the United States: A Sex- and Age-Adjusted Population Analysis. JAMA Dermatol. 2019 May 1; 155 (5): 627-629. PMCID: PMC6506879
16. Wei E X, Li S J, Mostaghimi A. Post-traumatic stress disorder in patients with autoimmune blistering diseases. Br J Dermatol. 2020 April; 182 (4): 1044-1045. PMID: 31545508
17. Vodo D, Sarig O, Sprecher E. The Genetics of Pemphigus Vulgaris. Front Med. 2018 Aug. 14; 5:226.
18. Ohyama B, Nishifuji K, Chan P T, Kawaguchi A, Yamashita T, Ishii N, Hamada T, Dainichi T, Koga H, Tsuruta D, Amagai M, Hashimoto T. Epitope spreading is rarely found in pemphigus vulgaris by large-scale longitudinal study using desmoglein 2-based swapped molecules. J Invest Dermatol. 2012 April; 132 (4): 1158-1168. PMID: 22277941
19. Bruhns P. Properties of mouse and human IgG receptors and their contribution to disease models. Blood. 2012 Jun. 14; 119 (24): 5640-5649. PMID: 22535666
20. Vidarsson G, Dekkers G, Rispens T. IgG subclasses and allotypes: from structure to effector functions. Front Immunol. 2014; 5:520. PMCID: PMC4202688
21. Yu J, Song Y, Tian W. How to select IgG subclasses in developing anti-tumor therapeutic antibodies. J Hematol Oncol. 2020 May 5; 13 (1): 45. PMCID: PMC7201658
22. Cong M, Tavakolpour S, Berland L, Glöckner H, Andreiuk B, Rakhshandehroo T, Uslu S, Mishra S, Clark L, Rashidian M. Direct N- or C-Terminal Protein Labeling Via a Sortase-Mediated Swapping Approach. Bioconjug Chem. 2021 Nov. 17; 32 (11): 2397-2406. PMID: 34748323
23. Guimaraes C P, Witte M D, Theile C S, Bozkurt G, Kundrat L, Blom A E M, Ploegh H L. Site-specific C-terminal and internal loop labeling of proteins using sortase-mediated reactions. Nat Protoc. 2013 September; 8 (9): 1787-1799. PMID: 23989673
24. Tsunoda K, Ota T, Aoki M, Yamada T, Nagai T, Nakagawa T, Koyasu S, Nishikawa T, Amagai M. Induction of pemphigus phenotype by a mouse monoclonal antibody against the amino-terminal adhesive interface of desmoglein 3. J Immunol. 2003 Feb. 15; 170 (4): 2170-2178. PMID: 12574390
25. Shimizu A, Ishiko A, Ota T, Saito H, Oka H, Tsunoda K, Amagai M, Nishikawa T. In vivo ultrastructural localization of the desmoglein 3 adhesive interface to the desmosome mid-line. J Invest Dermatol. 2005 May; 124 (5): 984-989. PMID: 15854040
26. Schmidt E, Dahnrich C, Rosemann A, Probst C, Komorowski L, Saschenbrecker S, Schlumberger W, Stöcker W, Hashimoto T, Brocker E-B, Recke A, Rose C, Zillikens D. Novel ELISA systems for antibodies to desmoglein 1 and 3: correlation of disease activity with serum autoantibody levels in individual pemphigus patients. Exp Dermatol. 2010 May; 19 (5): 458-463. PMID: 20163452
27. Takae Y, Nishikawa T, Amagai M. Pemphigus mouse model as a tool to evaluate various immunosuppressive therapies. Exp Dermatol. 2009 March; 18 (3): 252-260. PMID: 18643850
28. Aoki-Ota M, Tsunoda K, Ota T, Iwasaki T, Koyasu S, Amagai M, Nishikawa T. A mouse model of pemphigus vulgaris by adoptive transfer of naive splenocytes from desmoglein 3 knockout mice. Br J Dermatol. 2004 August; 151 (2): 346-354. PMID: 15327541
29. Amagai M, Tsunoda K, Suzuki H, Nishifuji K, Koyasu S, Nishikawa T. Use of autoantigen-knockout mice in developing an active autoimmune disease model for pemphigus. J Clin Invest. 2000 March; 105 (5): 625-631. PMCID: PMC292455
30. Lee J, Lundgren D K, Mao X, Manfredo-Vieira S, Nunez-Cruz S, Williams E F, Assenmacher C-A, Radaelli E, Oh S, Wang B, Ellebrecht C T, Fraietta J A, Milone M C, Payne A S. Antigen-specific B cell depletion for precision therapy of mucosal pemphigus vulgaris. J Clin Invest. 2020 Dec. 1; 130 (12): 6317-6324. PMCID: PMC7685721
31. Tham H L, Linder K E, Olivry T. Deep pemphigus (pemphigus vulgaris, pemphigus vegetans and paraneoplastic pemphigus) in dogs, cats and horses: a comprehensive review. BMC Vet Res. 2020 December; 16 (1): 457.
32. Mueller R S, Krebs I, Power H T, Fieseler K V. Pemphigus foliaceus in 91 dogs. J Am Anim Hosp Assoc. 2006 June; 42 (3): 189-196. PMID: 16611930
33. Levy B J, Mamo L B, Bizikova P. Detection of circulating anti-keratinocyte autoantibodies in feline pemphigus foliaceus. Vet Dermatol. 2020 October; 31 (5): 378-e100. PMID: 32372490
34. Preziosi D E. Feline Pemphigus Foliaceus. Vet Clin North Am Small Anim Pract. 2019 January; 49 (1): 95-104. PMID: 30227972
35. Vandenabeele S I J, White S D, Affolter V K, Kass P H, Ihrke P J. Pemphigus foliaceus in the horse: a retrospective study of 20 cases. Vet Dermatol. 2004 December; 15 (6): 381-388. PMID: 15585014
36. Zabel S, Mueller R S, Fieseler K V, Bettenay S V, Littlewood J D, Wagner R. Review of 15 cases of pemphigus foliaceus in horses and a survey of the literature. Vet Rec. 2005 Oct. 22; 157 (17): 505-509. PMID: 16244232
37. Goodale E. Pemphigus foliaceous. Can Vet J. 2019 March; 60 (3): 311-313. PMCID: PMC6380263
38. Olivry T, LaVoy A, Dunston S M, Brown R S, Lennon E M, Warren S J, Prisayanh P, Müller E J, Suter M M, Dean G A. Desmoglein-1 is a minor autoantigen in dogs with pemphigus foliaceus. Vet Immunol Immunopathol. 2006 Apr. 15; 110 (3-4): 245-255. PMID: 16293316
39. Bosch-Amate X, Iranzo P, Ivars M, Mascaró Galy J M, España A. Anti-Desmocollin Autoantibodies in Autoimmune Blistering Diseases. Front Immunol. 2021 Sep. 10; 12:740820.
40. Bizikova P, Dean G A, Hashimoto T, Olivry T. Cloning and establishment of canine desmocollin-1 as a major autoantigen in canine pemphigus foliaceus. Vet Immunol Immunopathol. 2012 Oct. 15; 149 (3-4): 197-207. PMID: 22884397
41. Bizikova P, Linder K E, Olivry T. Immunomapping of desmosomal and nondesmosomal adhesion molecules in healthy canine footpad, haired skin and buccal mucosal epithelia: comparison with canine pemphigus foliaceus serum immunoglobulin G staining patterns. Vet Dermatol. 2011 April; 22 (2): 132-142. PMID: 20738839
42. Miyakawa S, Nomura Y, Sakamoto T, Yamaguchi Y, Kato K, Yamazaki S, Nakamura Y. Structural and molecular basis for hyperspecificity of RNA aptamer to human immunoglobulin G. RNA. 2008 June; 14 (6): 1154-1163. PMCID: PMC2390795

### Example 4

```
mDsg3(EC13)-mFc (SEQ ID NO. 1):
EWVKFAKPCREREDNSRRNPIAKITSDFQKNQKITYRISGVGIDQPPFGIFVVDPNNGDIN
ITAIVDREETPSFLITCRALNALGQDVERPLILTVKILDVNDNPPIFSQTIFKGEIEENSASNS
LVMILNATDADEPNHMNSKIAFKIVSQEPAGMSMFLISRNTGEVRTLTSSLDREQISSYH
LVVSGADNDGTGLSTQCECSIKIKDVNDNFPVLRESQYSARIEENTLNAELLRFQVTDW
DEEYTDNWLAVYFFTSGNEGNWFEIETDPRTNEGILKVVKALDYEQVQSMQFSIAVRN
KAEFHQSVISQYRVQSTPVTIQVIDVREGIS-GGGGSGGS-
EPRGPTIKPCPPCKCPAPNLLGGPSVFIFPPKIKDVLMISLSPIVTCVVVDVSEDDPDVQIS
WFVNNVEVHTAQTQTHREDYNSTLRVVSALPIQHQDWMSGKEFKCKVNNKDLPAPIER
TISKPKGSVRAPQVYVLPPPEEEMTKKQVTLTCMVTDFMPEDIYVEWTNNGKTELNYK
NTEPVLDSDGSYFMYSKLRVEKKNWVERNSYSCSVVHEGLHNHHTTKSFSRTPGK-
GGGGSGGGGSDYKDDDDKLPETGGHHHHHH
hDsg3(EC13)-hFc (SEQ ID NO. 2):
EWVKFAKPCREGEDNSKRNPIAKITSDYQATQKITYRISGVGIDQPPFGIFVVDKNTGDIN
ITAIVDREETPSFLITCRALNAQGLDVEKPLILTVKILDINDNPPVFSQQIFMGEIEENSASN
SLVMILNATDADEPNHLNSKIAFKIVSQEPAGTPMFLLSRNTGEVRTLTNSLDREQASSY
RLVVSGADKDGEGLSTQCECNIKVKDVNDNFPMFRDSQYSARIEENILSSELLRFQVTDL
DEEYTDNWLAVYFFTSGNEGNWFEIQTDPRTNEGILKVVKALDYEQLQSVKLSIAVKNK
AEFHQSVISRYRVQSTPVTIQVINVREGIA-GGGGS-
EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSLSLSPGK-
GGGGSDYKDDDDKLPETGGHHHHHH
mDsg3(EC14)-mFc
(SEQ ID NO. 3)
EWVKFAKPCREREDNSRRNPIAKITSDFQKNQKITYRISGVGIDQPPFGIFVVDPNNGDIN
ITAIVDREETPSFLITCRALNALGQDVERPLILTVKILDVNDNPPIFSQTIFKGEIEENSASNS
LVMILNATDADEPNHMNSKIAFKIVSQEPAGMSMFLISRNTGEVRTLTSSLDREQISSYH
LVVSGADNDGTGLSTQCECSIKIKDVNDNFPVLRESQYSARIEENTLNAELLRFQVTDW
DEEYTDNWLAVYFFTSGNEGNWFEIETDPRTNEGILKVVKALDYEQVQSMQFSIAVRN
KAEFHQSVISQYRVQSTPVTIQVIDVREGISFRPPSKTFTVQRGVSTNKLVGYILGTYQAT
DEDTGKAASSVRYVLGRNDGGLLVIDSKTAQIKFVKNIDRDSTFIVNKTISAEVLAIDEN
TGKTSTGTIYVEVPSFNENGSPTIKPSPPSKSPAPNLLGGPSVFIFPPKIKDVLMISLSPIVTC
VVVDVSEDDPDVQISWFVNNVEVHTAQTQTHREDYNSTLRVVSALPIQHQDWMSGKEF
KCKVNNKDLPAPIERTISKPKGSVRAPQVYVLPPPEEEMTKKQVTLTCMVTDFMPEDIY
VEWTNNGKTELNYKNTEPVLDSDGSYFMYSKLRVEKKNWVERNSYSCSVVHEGLHNH
HTTKSFSRTPGKEFDYKDDDDKGSLPETGGHHHHHH
hDsg3(EC14)-hFc
(SEQ ID NO. 4)
EWVKFAKPCREGEDNSKRNPIAKITSDYQATQKITYRISGVGIDQPPFGIFVVDKNTGDIN
ITAIVDREETPSFLITCRALNAQGLDVEKPLILTVKILDINDNPPVFSQQIFMGEIEENSASN
SLVMILNATDADEPNHLNSKIAFKIVSQEPAGTPMFLLSRNTGEVRTLTNSLDREQASSY
RLVVSGADKDGEGLSTQCECNIKVKDVNDNFPMFRDSQYSARIEENILSSELLRFQVTDL
DEEYTDNWLAVYFFTSGNEGNWFEIQTDPRTNEGILKVVKALDYEQLQSVKLSIAVKNK
AEFHQSVISRYRVQSTPVTIQVINVREGIAFRPASKTFTVQKGISSKKLVDYILGTYQAIDE
DTNKAASNVKYVMGRNDGGYLMIDSKTAEIKFVKNMNRDSTFIVNKTITAEVLAIDEYT
GKTSTGTVYVRVPDFNDNGGGGSEPKSADKTHTSPPAPAPELLGGPSVFLFPPKPKDTL
MISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSV
MHEALHNHYTQKSLSLSPGKGEFDYKDDDDKLPETGHHHHHH
hDsg3(EC1-5)-hFc
(SEQ ID NO. 29)
EWVKFAKPCREREDNSRRNPIAKITSDFQKNQKITYRISGVGIDQPPFGIFVVDPNNGDIN
ITAIVDREETPSFLITCRALNALGQDVERPLILTVKILDVNDNPPIFSQTIFKGEIEENSASNS
LVMILNATDADEPNHMNSKIAFKIVSQEPAGMSMFLISRNTGEVRTLTSSLDREQISSYH
LVVSGADNDGTGLSTQCECSIKIKDVNDNFPVLRESQYSARIEENTLNAELLRFQVTDW
DEEYTDNWLAVYFFTSGNEGNWFEIETDPRTNEGILKVVKALDYEQVQSMQFSIAVRN
KAEFHQSVISQYRVQSTPVTIQVIDVREGISFRPPSKTFTVQRGVSTNKLVGYILGTYQAT
DEDTGKAASSVRYVLGRNDGGLLVIDSKTAQIKFVKNIDRDSTFIVNKTISAEVLAIDEN
TGKTSTGTIYVEVPSFNENCPSVVLEKKDICTSSPSVTLSVRTLDRGKYTGPYTVSLEEQP
LKLPVMWTITTLNATSALLQAQQQVSPGVYNVPVIVKDNQDGLCDTPESLTLTVCQCD
DRSMCRAPIPSREPNTYEFGSEPKSADKTHTCPPCPAPELLGGPSVFLFPPKPKDTLMISR
TPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHE
ALHNHYTQKSLSLSPGKGSDYKDDDDKLPETGHHHHHH
mDsg3(EC1-5)-mFc
(SEQ ID NO. 30)
EWVKFAKPCREREDNSRRNPIAKITSDFQKNQKITYRISGVGIDQPPFGIFVVDPNNGDIN
ITAIVDREETPSFLITCRALNALGQDVERPLILTVKILDVNDNPPIFSQTIFKGEIEENSASNS
LVMILNATDADEPNHMNSKIAFKIVSQEPAGMSMFLISRNTGEVRTLTSSLDREQISSYH
LVVSGADNDGTGLSTQCECSIKIKDVNDNFPVLRESQYSARIEENTLNAELLRFQVTDW
DEEYTDNWLAVYFFTSGNEGNWFEIETDPRTNEGILKVVKALDYEQVQSMQFSIAVRN
KAEFHQSVISQYRVQSTPVTIQVIDVREGISFRPPSKTFTVQRGVSTNKLVGYILGTYQAT
DEDTGKAASSVRYVLGRNDGGLLVIDSKTAQIKFVKNIDRDSTFIVNKTISAEVLAIDEN
TGKTSTGTIYVEVPSFNENCPSVVLEKKDICTSSPSVTLSVRTLDRGKYTGPYTVSLEEQP
LKLPVMWTITTLNATSALLQAQQQVSPGVYNVPVIVKDNQDGLCDTPESLTLTVCQCD
DRSMCRAPIPSREPNTYEFGSPRGPTIKPCPPCKCPAPNLLGGPSVFIFPPKIKDVLMISLSP
IVTCVVVDVSEDDPDVQISWFVNNVEVHTAQTQTHREDYNSTLRVVSALPIQHQDWMS
GKEFKCKVNNKDLPAPIERTISKPKGSVRAPQVYVLPPPEEEMTKKQVTLTCMVTDFMP
EDIYVEWTNNGKTELNYKNTEPVLDSDGSYFMYSKLRVEKKNWVERNSYSCSVVHEG
LHNHHTTKSFSRTPGKGSDYKDDDDKLPETGHHHHHH
hDsg1(EC1-4)-hFc
(SEQ ID NO. 31)
EWIKFAAACREGEDNSKRNPIAKIHSDCAANQQVTYRISGVGIDQPPYGIFVINQKTGEIN
ITSIVDREVTPFFIIYCRALNSMGQDLERPLELRVRVLDINDNPPVFSMATFAGQIEENSN
ANTLVMILNATDADEPNNLNSKIAFKIIRQEPSDSPMFIINRNTGEIRTMNNFLDREQYGQ
YALAVRGSDRDGGADGMSAECECNIKILDVNDNIPYMEQSSYTIEIQENTLNSNLLEIRVI
DLDEEFSANWMAVIFFISGNEGNWFEIEMNERTNVGILKVVKPLDYEAMQSLQLSIGVR
NKAEFHHSIMSQYKLKASAISVTVLNVIEGPVFRPGSKTYVVTGNMGSNDKVGDFVAT
DLDTGRPSTTVRYVMGNNPADLLAVDSRTGKLTLKNKVTKEQYNMLGGKYQGTILSID
DNLQRTCTGTININIQSFGNDDGSEPKSCDKTHTCPPCSAPELLGGPSVFLFPPKPKDTLM
ISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSV
MHEALHNHYTQKSLSLSPGKGGGGSEFDYKDDDDKGSLPETGGHHHHHH
mDsg1(EC1-4)-mFc
(SEQ ID NO. 32)
EWIKFAAACREGEDNSKRNPIAKIHSDCAANQPVTYRISGVGIDQPPYGIFIINQKTGEINI
TSIVDREVTPFFIIYCRALNAQGQDLENPLELRVRVMDINDNPPVFSMTTFLGQIEENSNA
NTLVMKLNATDADEPNNLNSMIAFKIIRQEPSDSPMFIINRKTGEIRTMNNFLDREQYSQ
YSLVVRGSDRDGGADGMSAESECSITILDVNDNIPYLEQSSYDIEIEENALHSQLVQIRVI
DLDEEFSDNWKAIIFFISGNEGNWFEIEMNERTNVGTLKVVKPLDYEAMKNLQLSIGVR
NVAEFHQSIISQYRLTATMVTVTVLNVIEGSVFRPGSKTFVVDSRMEANHRVGEFVATD
LDTGRASTNVRYEMGNNPENLLVVDSRTGIITLRNRVTMEQYQRLNGEYKGTVLSIDDS
LQRTCTGTIVIELSGTGWVPGSGSPRGPTIKPCPPCKCPAPNLLGGPSVFIFPPKIKDVLMI
SLSPIVTCVVVDVSEDDPDVQISWFVNNVEVHTAQTQTHREDYNSTLRVVSALPIQHQD
WMSGKEFKCKVNNKDLPAPIERTISKPKGSVRAPQVYVLPPPEEEMTKKQVTLTCMVT
DFMPEDIYVEWTNNGKTELNYKNTEPVLDSDGSYFMYSKLRVEKKNWVERNSYSCSV
VHEGLHNHHTTKSFSRTPGKEFDYKDDDDKGSLPETGGHHHHHH
Human Dsg3 Amino Acid Sequence
(SEQ ID NO. 5)
                10 20 30 40 50
MMGLFPRTTG ALAIFVVVIL VHGELRIETK GQYDEEEMTM QQAKRRQKRE
                60 70 80 90 100
WVKFAKPCRE GEDNSKRNPI AKITSDYQAT QKITYRISGY GIDQPPFGIF
              110 120 130 140 150
VVDKNTGDIN ITAIVDREET PSFLITCRAL NAQGLDVEKP LILTVKILDI
              160 170 180 190 200
NDNPPVFSQQ IFMGEIEENS ASNSLVMILN ATDADEPNHL NSKIAFRIVS
              210 220 230 240 250
QEPAGTPMFL LSRNTGEVRT LTNSLDREQA SSYRLVVSGA DKDGEGLSTQ
              260 270 280 290 300
CECNIKVKDV NDNFPMFRDS QYSARIEENI LSSELLRFQV TDLDEEYTDN
              310 320 330 340 350
WLAVYFFTSG NEGNWPEIQT DPRTNEGILK VVKALDYEQL QSVKLSIAVK
              360 370 380 390 400
NKAEFHQSVI SRYRVQSTPV TIQVINVREG IAFRPASKTF TVQKGISSKK
              410 420 430 440 450
LYDYILGTYQ AIDEDTNKAA SNVKYVMGRN DGGYLMIDSK TAEIKFVKNM
              460 470 480 490 500
NRDSTFIVNK TITAEVLAID EYTGKTSTGT VYVRVPDFND NCPTAVLEKD
              510 520 530 540 550
AVCSSSPSVV VSARTLNNRY TGPYTFALED QPVKLPAVWS ITTLNATSAL
              560 570 580 590 600
LRAQEQIPPG VYHISLVLTD SQNNRCEMPR SLTLEVCQCD NRGICGTSYP
              610 620 630 640 650
TYSPGTRYGR PHSGRLGPAA IGLLLLGLLL LLLAPLLLLT CDCGAGSTGG
              660 670 680 690 700
VTGGFIPVPD GSEGTIHQWG IEGAHPEDKE ITNICVPPVT ANGADFMESS
              710 720 730 740 750
EVCTNTYARG TAVEGTSGME MTTKLGAATE SGGAAGFATG TVSGAASGFG
              760 770 780 790 800
AATGVGICSS GQSGTMRTRH STGGTNKDYA DGAISMNFLD SYFSQKAFAC
              810 820 830 840 850
AEEDDGQEAN DCLLIYDNEG ADATGSPVGS VGCCSFIADD LDDSFLDSLG
              860 870 880 890 900
PKFKKLAEIS LGVDGEGKEV QPPSKDSGYG IESCGHPIEV QQTGFVKCQT
              910 920 930 940 950
LSGSQGASAL STSGSVQPAV SIPDPLQHGN YLVTETYSAS GSLVQPSTAG
              960 970 980 990
FDPLLTQNVI VTERVICPIS SVPGNLAGPT QLRGSHTMLC TEDPCSRLI
Human Dsg1 Amino Acid Sequence, Isoform 1
(SEQ ID NO. 6)
                10 20 30 40 50
MMGLFPRTTG ALAIFVVVIL VHGELRIETK GQYDEEEMTM QQAKRRQKRE
                60 70 80 90 100
WVKFAKPCRE GEDNSKRNPI AKITSDYQAT QKITYRISGV GIDQPPFGIF
              110 120 130 140 150
VVDKNTGDIN ITAIVDREET PSFLITCRAL NAQGLDVEKP LILTVKILDI
              160 170 180 190 200
NDNPPVFSQQ IFMGEIEENS ASNSLVMILN ATDADEFNHL NSKIAFKIVS
              210 220 230 240 250
QEPAGTPMFL LSRNTGEVRT LTNSLDREQA SSYRLVVSGA DKDGEGLSTQ
              260 270 280 290 300
CECNIKVKDV NDNFPMFRDS QYSARIEENI LSSELLRFQV TDLDEEYTDN
              310 320 330 340 350
WLAVYFFTSG NEGNWFEIQT DPRTNEGILK VVKALDYEQL QSVKLSIAVK
              360 370 380 390 400
NKAEFHQSVI SRYRVQSTPV TIQVINVREG IAFRPASKTF TVQKGISSKK
              410 420 430 440 450
LVDYILGTYQ AIDEDTNKAA SNVKYVMGRN DGGYLMIDSK TAEIKFVKNM
              460 470 480 490 500
NRDSTFIVNK TITAEVLAID EYTGKTSTGT VYVRVPDFND NCPTAVLEKD
              510 520 530 540 550
AVCSSSPSVV VSARTLNNRY TGPYTFALED QPVKLPAVWS ITTLNAPSAL
              560 570 580 590 600
LRAQEQIPPG VYHISLVLTD SQNNRCEMPR SLTLEVCQCD NRGICGTSYP
              610 620 630 640 650
TTSPGTRYGR PHSGRLGPAA IGLLLLGLLL LLLAPLLLLT CDCGAGSTGG
              660 670 680 690 700
VTGGFIPVPD GSEGTIHQWG IEGAHPEDKE ITNICVPPVT ANGADFMESS
              710 720 730 740 750
EVCTNTYARG TAVEGTSGME MTTKLGAATE SGGAAGFATG TVSGAASGFG
              760 770 780 790 800
AATGVGICSS GQSGTMRTRH STGGTNKDYA DGAISMNFLD SYFSQKAFAC
              810 820 830 840 850
AEEDDGQEAN DCLLIYDNEG ADATGSPVGS VGCCSFIADD LDDSFLDSLG
              860 870 880 890 900
PKFKLLAEIS LGVDGEGKEV QPPSKDSGYG IESCGHPIEV QQTGFVKCQT
              910 920 930 940 950
LSGSQGASAL STSGSVQPAV SIPDPLQHGN YLVTETYSAS GSLVQPSTAG
              960 970 980 990
FDPLLTQNVI VTERVICPIS SVPGNLAGPT QLRGSHTMLC TEDPCSRLI
Human Dsg1 Amino Acid Sequence, Isoform 2
(SEQ ID NO. 7)
                10 20 30 40 50
MQDLGGGERM TGFELTEGVK TSGMPEICQE YSGTLRRNSM RECREGGLNM
                60 70 80 90 100
NFMESYFCQK AYAYADEDEG RPSNDCLLIY DIEGVGSPAG SVGCCSFIGE
              110 120 130 140 150
DLDDSFLDTL GPKFKKLADI SLGKESYPDL DPSWPPQSTE PVCLPQETEP
              160 170 180 190 200
VVSGHPPISP HFGTTTVISE STYPSGPGVL HPKPILDPLG YGNVTVTESY
              210 220 230 240 250
TTSDTLKPSV HVHDNRPASN VVVTERVVGP ISGADLHGML EMPDLRDGSN
              260 270 280 290 300
VIVTERVIAP SSSLPTSLTI HHPRESSNVV VTERVIQPTS GMIGSLSMHP
              310 320 330 340 350
ELANAHNVIV TERVVSGAGV TGISGTTGIS GGIGSSGLVG TSMGAGSGAL
              360 370 380 390 400
SGAGISGGGI GLSSLGGTAS IGHMRSSSDH HFNQTIGSAS PSTARSRITK
YSTVQYSK
```

### Example 5

### Pemphigus Vulgaris (PV)

Devastating chronic autoimmune disease, can be characterized by painful blistering of the skin and mucosa
Can be life-threatening with a mortality rate of ˜5-15%. If untreated, 8 out of 10 die within a year
US prevalence: 30,000-40,000 cases; WW incidence 1-10 cases/1M people
Usually not genetic, and triggered later in life by environmental factors
Caused by production of autoAbs against desmoglein 3 (Dsg3), presented on B-Cells
Dsg3 is a calcium-binding transmembrane glycoprotein component of desmosomes in vertebrate epithelial cells
Desmosomes are cell-cell junctions between epithelial, myocardial, and certain other cell types
No Cure
Therapeutic strategies
Systemic steroid use and/or immunosuppressive drugs
Anti-CD20 antibodies (e.g., rituximab) are first line option (targets all B cells)
Off-target immunosuppressive effects including increased risk of infection
High risk of relapse after treatment
Anti-Dsg3 presenting B-cell depletion:
Cabaletta Bio: potential treatment based on Chimeric AutoAntibody Receptor T-Cells (CAAR-T)-IND cleared
Additional new strategies are needed

### Selective Depletion of Anti-Dsg3-Ab Presenting B-Cells—Two Non-Limiting, Exemplary Approaches

### A) “Fc-Mediated”

Similar to rituximab but now selective for Dsg3-B-cells (safer/fewer side reactions)
Circumvents the technical challenges/cost of CAAR-T (autologous cell therapy)

### B) “BCR-Effector Cell Engagers, Including BCR-T Cell Engagers”

Selective
Versatile-autoantibody/immune-complex mediated diseases to known antigenic targets
bullous pemphigoid, mucous membrane pemphigoid, epidermolysis bullosa acquisita, linear IgA bullous dermatosis
subtypes of lupus, scleroderma, Goodpasture's disease, Graves' disease
shown in vitro binding of Dsg3-Fc fusion to mouse Fcγ receptor of mouse macrophages, and killing of 94% of target B-cells in 48 h (@10 nM)
Created cell line to produce the mouse protein construct, are working on in vivo model
We can show in vitro effect on peripheral blood mononuclear cells (PBMCs) from PV patients with autoreactive B cells to Dsg3 protein (different disease phases)

### Non-Limiting Examples of Subject Matter Described Herein

Fc-Dsg3 fusion construct sequence
treatment of PV

### Example 6

### Non-Limiting, Exemplary Methodology for Attachment of a Therapeutic Moiety, an Imaging Moiety, a Capturing Moiety, or a Combination Thereof

In certain embodiments, sortase technology can be used to site-specifically attach any biomolecule of interest at the C-terminus of a protein. In certain clinical embodiments, biomolecules of interest can be attached non-specifically as well, for example, via maleimide reaction with free cysteines, or NHS-active groups with free amines of lysine side-chains.

Certain embodiments can comprise multiple antigens on single fusion protein. An embodiment comprises a knob-into-hole Fc. Under one embodiment, an antigen is placed on the hole, and a second antigen on the knob. Such embodiments permit inclusion of two major antigens in one drug. By way of example, embodiments can comprise an Dsg3-knob, and an Dsg1-hole or vice versa. Embodiments can comprise specific extracellular (EC) domains of Dsg1 or Dsg3, for example, as antigens included in these therapeutics. Such embodiments can be useful in the treatment for pemphigus patients.

### Various Embodiments can be Utilized in the Treatment of Autoimmune Disorders

Exemplary autoimmune disorders are provided in U.S. Pat. No. 7,332,168, which is hereby incorporated by reference in its entirety. Non-limiting examples of autoimmune disorders (along with exemplary auto-antigens suitable for use in certain therapeutic constructs) are provided below:
Paraneoplastic pemphigus (IgG Abs against predominantly plakins (envoplakin, periplakin, desmoplakin I, desmoplakin II, epiplakin, plectin, BP230), cadherins (Dsg3, Dsg1, Dsc1, Dsc2, Dsc3), α2-macroglobulin-like molecules)
IgA pemphigus: IgA against Dsg1, Dsg3, Dsc1, Dsc2, Dsc3
Pemphigus vegetans: IgG against Dsg3, Dsg1, Dsc3
Pemphigus erythematosus: IgG Dsg1
Pemphigus herpetiformis: IgG Dsg1, Dsg3, Dsc1, Dsc3
Drug-induced pemphigus: IgG Dsg1, Dsg3
Bullous Pemphigoid: Anti IgG against BP180 (NC16A domain), BP230 Lichen planus pemphigoides: IgG against BP180 (NC16A) domain, BP230
Pemphigoid gestation: Anti IgG against BP180 (NC16A domain; 90% of cases), BP230 (less prevalent)
Linear IgA dermatosis: IgA against BP180 NC16A/LABD97/LAD-1 domains, and BP 230
Mucous membrane pemphigoid: IgG/IgA against BP180 [NC16A domain/C-terminal epitopes], laminin 332, BP230, α6β4 integrin, and collagen VII
Anti-laminin gamma 1/p200 pemphigoid: IgG against Laminin gamma1 (p200)
Epidermolysis bullosa acquisita: IgG against Type VII collagen
Dermatitis herpetiformis: IgA/G against Epidermal transglutaminase, tissue transglutaminase, endomysium, deamidated gliadin
Myasthenia Gravis
Grave's Disease
Immune Thrombocytopeniaurpura
Psoriasis
Vitiligo
Systemic Lupus Erythematosus
Type 1 diabetes
Rheumatoid Arthritis
Multiple sclerosis
Crohn's Disease
Ulcerative colitis
Addison's disease
Sjögren's syndrome
Hashimoto's thyroiditis
Autoimmune vasculitis
Pernicious anemia
Celiac disease
Ankylosing spondylitis
Behcet's syndrome
Congenital adrenal hyperplasia
Goodpasture syndrome
Hereditary hemochromatosis
Idiopathic membranous glomerulonephritis
Narcolepsy
Sarcoidosis

Autoimmune kidney diseases, for example, see the 124 autoantigens as described in Zhang W, Rho J-h, Roehrl M W, Roehrl M H, Wang J Y (2019) A repertoire of 124 potential autoantigens for autoimmune kidney diseases identified by dermatan sulfate affinity enrichment of kidney tissue proteins. PLOS ONE 14 (6), which is hereby incorporated by reference in its entirety.

### Example 7

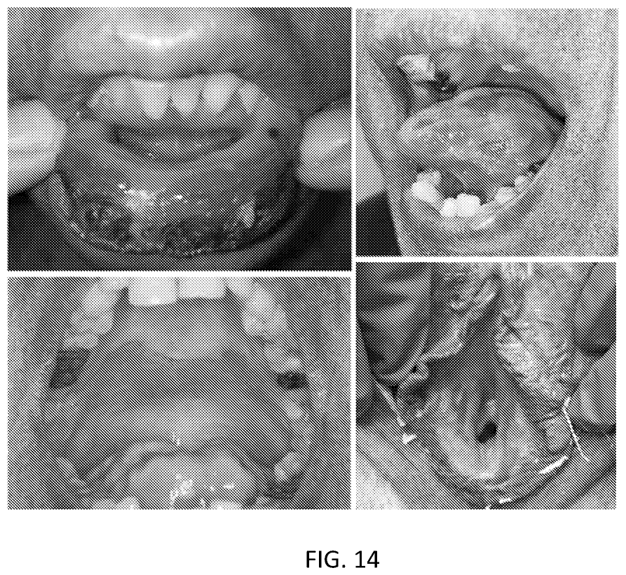

Background and Significance. Pemphigus vulgaris (PV) is a devastating autoimmune disease, which can be characterized by painful blistering of the skin and mucosa¹⁻⁴ Desmogleins, a part of the cadherin family, are the primary cellular glue between keratinocytes in stratified epithelium. Autoantibodies against desmoglein molecules inhibit their adhesive function resulting in loss of cell-to-cell adhesion, known as acantholysis^{5,6}. Additionally, the autoantibodies' interference in cell-to-cell adhesion leads to cell signaling pathways that augment the pathological autoimmune response and contribute to acantholysis^{2,3,7}. Mucosal-dominant PV is caused by the production of autoantibodies against desmoglein-3 (Dsg3), resulting in painful mucosal erosions^{8,2,9} (FIG. 14). Oral mucosal erosions are particularly painful and troubling for patients causing difficulty in eating and drinking. Mucosal erosions can occur in other mucosal services as well including nasal, vaginal, perianal, laryngeal/esophageal, urogenital, and conjunctival mucosa (FIG. 14). There is no cure for PV; treatment strategies typically include systemic steroids and immunosuppressive drugs¹⁰⁻²². Anti-Dsg3 antibodies from autoreactive B cells are the primary drivers of mucosal-dominant-PV^{23,24}. The direct role of pathogenic autoantibodies in pemphigus has been established and monovalent autoantibodies have been shown to be sufficient to cause the disease^{8,25,26} While anti-CD20 antibodies (e.g., rituximab) have become the first-line option for moderate-to-severe disease^{11,4,27-30}, it is associated with off-target immunosuppressive effects, including an increased risk of infection (albeit lower than traditional immunosuppressive agents), depletion of B cells, obtunding the humoral response to vaccination or new infections for 6 or more months, and a high risk of relapse after treatment^{29,31-34}. Treatment options including neonatal Fc receptor antagonists^{35,36}, blocking antibodies against B cell-activating factor receptor (BAFF-R)³⁷, and Bruton's Tyrosine Kinase inhibitors (BTKi)^{38,39}, some currently in clinical trials, would not circumvent the off-target immunosuppressive effects of obtunding the global humoral immunity and carry a high risk of severe hypogammaglobulinemia, particularly in the case of neonatal Fc receptor antagonists⁴⁰. Additionally, early animal study of BTKi show efficacy, but not a complete cure for canine pemphigus foliaceus (a superficial variant of pemphigus driven by anti-desmocollin and anti-desmoglein autoantibodies in dogs); BTKi was not able to completely suppress the production of autoantibody in all canines, which is key to disease remission and cure³⁹. Relapse in PV can be due to the expansion of the same autoreactive cells not depleted during initial treatment⁴¹⁻⁴³. Thus, complete depletion of autoreactive B cells can offer a cure. Early-stage cell-based therapies, such as autologous polyclonal regulatory T cells⁴⁴ and chimeric autoantigen receptor (CAAR) T cells⁴⁵, if proven effective, cannot be globally immunosuppressive. However, cell-based therapies can be personalized to an individual patient, and have specific requirements for manufacturing, storage, and transport, which cannot be feasible in all hospitals and clinics treating this disease group. The targeting of Inducible Co-Stimulator (ICOS) molecule, a costimulatory receptor expressed on T cells (which influences the activity of T follicular helper cells that play critical roles in development of anti-Dsg3 pathogenic B cells), via anti-ICOS antibody has shown results in delaying the progression of PV in animal models⁴⁶; however, this is a less direct way of targeting anti-Dsg3 antibody production compared to targeting anti-Dsg3 B cells themselves, without wishing to be bound by theory, can only delay the progression of the disease, and may have negative impacts on the production of other protective antibodies. The development of a targeted treatment that precisely depletes autoreactive cells while preserving the protective immunity is an unmet need and will be lifesaving for many patients.

Dsg3 and Dsg1 have different expression patterns in skin and mucosa¹. Dsg3 can be expressed through the mucosa and in the deeper layers of the adult cutaneous skin; Dsg1 and cadherin are unable to compensate for the loss of Dsg3 function in the mucosa in mucosal-dominant PV. Anti-desmoglein autoantibodies play a pathogenic role in inducing loss of adhesion between keratinocytes and formation of blisters and erosions. Experimental models have shown that Dsg3 and Dsg1 autoantibodies are directly pathogenic^{24,25,47}. Anti-Dsg1 and anti-Dsg3 autoantibody levels can correlate with disease severity⁴⁸. Perilesional autoreactive B and T lymphocytes that work together to produce autoreactive antibodies are the cellular players in disease pathogenesis⁴⁹; relapse can be correlated with the return of autoreactive T and B cells, which work together to cause disease phenotype. Remission is induced by lowering or depletion of autoreactive antibodies targeting desmoglein. Relapse is higher in patients who have incomplete depletion of autoreactive B-cells⁵⁰. Thus, complete targeted depletion of desmoglein-recognizing autoreactive B cells, the direct effector cell of the disease phenotype, is the key to treating this group of autoantibody-driven diseases; autoreactive T cells alone, without autoreactive B cells, can be not able to induce disease phenotype, as suppression of autoreactive B cells is critical to maintaining remission^{43,51}.

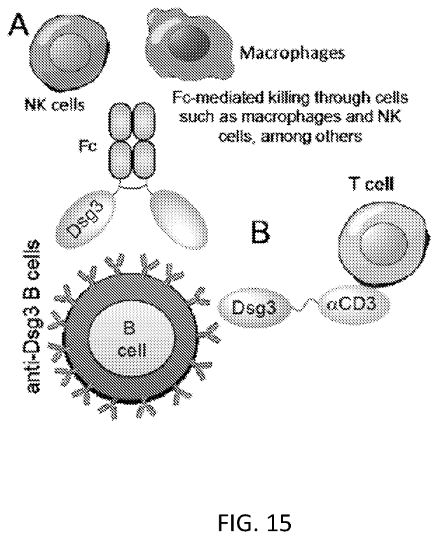

We can tackle this issue by leveraging protein-engineering techniques and generating new tools to precisely target and deplete pathogenic B cells, without affecting the global B cell pool. We can describe mucosal-dominant PV that is driven by anti-Dsg3 autoreactive B cells. In a mechanism analogous to existing therapeutic monoclonal antibodies, including rituximab²⁹, without wishing to be bound by theory, a Dsg3 protein fused to an Fc portion of an antibody can effectively and specifically mediate the killing of the autoreactive Dsg3-recognizing B cells. The Dsg3-portion of the construct binds to the anti-Dsg3 B-cell receptor (BCR) of autoreactive B cells. The Fc portion then induces Fc-mediated killing of the pathogenic B cells recognizing Dsg3 by recruitment of phagocytic and natural killer (NK) cells (FIG. 15 panel A). We have created murine and human Dsg3-Fc fusions and shown that these constructs bind to their targets with specificity and affinity. Additionally, our data shows that the engineered proteins mediate the killing of established Dsg3-specific B cells in vitro and in vivo with efficacy (full response observed in the in vivo animal models). As an alternative approach, we can use a Dsg3 protein fused to an anti-CD3e scFv to direct and engage T cells to kill Dsg3-recognizing pathogenic B cells, in a mechanism analogous to existing bi-specific T cell engager therapeutics in oncologic treatments (FIG. 15 panel B)^{52,53}.

Without wishing to be bound by theory, the compositions and methods described herein can provide a platform for the treatment of not only for mucosal-dominant PV, other variant of pemphigus, but also antibody-driven autoimmune diseases with known antigens.

There is no cure for pemphigus and treatments are globally immunosuppressive. The Fc-mediated approach described herein (and our alternative T cell-mediated approach), which allows precise targeting and depletion of Dsg3-autoreactive pathogenic B cells without impacting normal B cells, is unique. The data presented herein indicate that the compositions and methods described herein can transform the treatment of not only mucosal-dominant PV, but also opens avenues for the development of treatments for autoantibody/immune-complex mediated diseases with known antigenic targets, ranging from other autoimmune blistering diseases (e.g., mucocutaneous pemphigus vulgaris, pemphigus foliaceus, bullous pemphigoid, mucous membrane pemphigoid, epidermolysis bullosa acquisita, linear IgA bullous dermatosis), subtypes of lupus, scleroderma, Goodpasture's disease, Graves' disease, to immune-mediated vasculitis. We believe the proposed therapeutics can be considered a ground-breaking addition to the therapeutic toolbox for autoimmune disease.

The biological activity can be established using standardized in vitro assays. Extensive ex vivo biodistribution and metabolic fate analyses can be performed. For each set of experiments, at least about 10 mice per group can be used to enable statistical analysis and account for the inherent animal-to-animal variability. Where applicable, statistical analyses can be performed using an unpaired Student 2-tailed t test with significance level α<=0.05. Survival analyses can be performed using a log-rank (Mantel-Cox) test. Animals can be age-matched within each experiment. Experiments can include both male and female mice to account for gender differences in response to the therapeutics. The therapeutic studies in animals can be carried out in a double-blinded fashion to ensure that observer bias is avoided. Non-commercial biologics and chemicals can be authenticated.

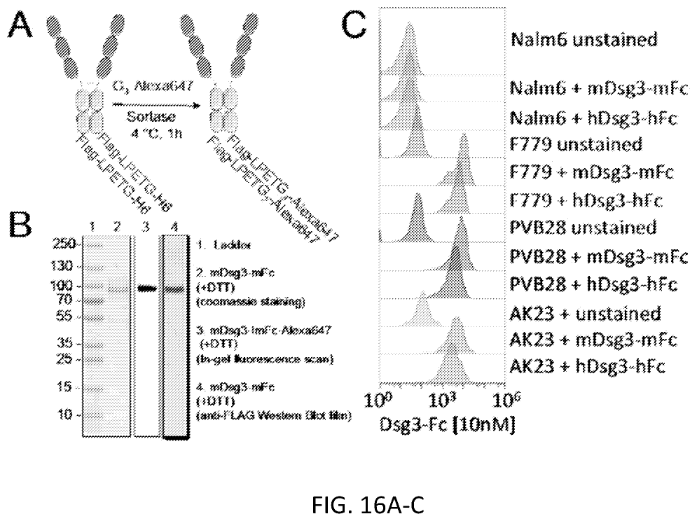

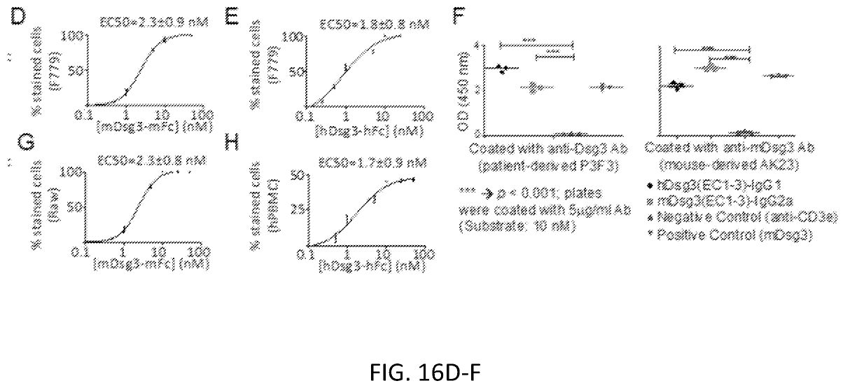

Site-specific modification of proteins. Sortase, a bacterial transpeptidase, can be used to modify proteins at the C-terminus (FIG. 16 panel A)^{54,55}. The enzyme has a short recognition sequence (LPETG), cleaves between the T and G to form an intermediate thioester, allowing resolution by a nucleophilic reaction with a Gly3-containing substrate. The reaction can provide near-quantitative yield and can be rapid, robust, and reproducible. We have used sortase to install functionalities onto proteins, such as fluorophores, polymers, and radioisotopes⁵⁶⁻⁶¹. The sortase-mediated site-specific labeling ensures the binding capacity of proteins will not be compromised after labeling, and therefore, here, we will use the sortase technology to modify proteins.

Immunogenicity of Dsg3 ectodomains. Studies on PV patients have shown that 91%, 71%, 51%, 19% and 12% of patients have autoantibodies against ectodomains EC1, EC2, EC3, EC4 and EC5 (FIG. 13), respectively⁶². To establish the approach, we can use Dsg3-EC1-3, as it covers the majority of the physiological anti-Dsg3 antibodies in PV patients and its production is less complex than the full-size EC1-5 construct. Furthermore, non-limiting, exemplary results (below) show similar killing of autoreactive B-cells independent of the ectodomain targeted. Once established, we will make EC1-4 and 1-5 constructs, which can be more relevant when considering clinical translation, and to cover the full spectrum of anti-Dsg3 reactive pathogenic B cells.

Anti-Dsg3 cell lines and antibodies. We can use three cell lines and four anti-Dsg3 antibodies: 1) AK23 hybridoma cells, which produces anti-mouse Dsg3 antibodies that induces PV in mice63. The AK23 antibody recognizes EC1 domain and cross-react with human Dsg3-EC1; 2 and 3) Nalm-6 F779 and Nalm-6 PVB28 cancer cell lines. Nalm-6 is a B cell precursor leukemia cell line, and F779 and PVB28 are the Nalm-6 cells engineered to express patient-derived anti-Dsg3 antibodies that recognize Dsg3-EC1 and Dsg3-EC2 domains, respectively⁴⁵. Furthermore, we have obtained the plasmids for four anti-human Dsg3 antibodies (P3F3, P1F5, P5D4 and P5G6) identified from a PV patient⁶⁴. The P3F3 antibody is an IgG1 antibody that binds specifically to human Dsg3-EC1 domain, inducing strong acantholysis when added to human keratinocytes⁶⁴. P1F5, P5D4, and P5G6 antibodies bind to the Dsg3-EC2, Dsg3-EC4 and Dsg3-EC1 domains respectively and together induce strong acantholysis⁶⁴. The combined monoclonal antibodies cause higher dissociation of keratinocytes than any single one of them.

To develop Dsg3-Fc fusion proteins to target and mediate the killing of anti-Dsg3 autoreactive B cells in in-vitro settings.

Development and characterization of mDsg3-mFc and hDsg3-hFc constructs. As discussed herein, to establish the approach we developed the Dsg3-EC1-3 domains. Accordingly, we have made both mouse and human Fc-fusion proteins: mDsg3-EC1-3-IgG2a and hDsg3-EC1-3-IgG1 (from now on referred to as mDsg3-mFc, and hDsg3-hFc). Human IgG1 and mouse IgG2a were chosen for their high Fc-mediated killing activities⁶⁵⁻⁶⁷. The proteins were engineered to have a FLAG tag, a sortase recognition motif and a His6 tag at the C-terminus (FIG. 16 panel A). The FLAG, an 8 amino acid sequence (DYKDDDDK), can be used for Western Blot and flow cytometry analyses, and subsequently for analysis of the constructs' metabolic fate and its pharmacokinetics when used in vivo. The His6 tag was used for purification and the sortase tag (LPETG) was used to site-specifically introduce modifications. We made the plasmids for both constructs following established cloning techniques and confirmed the final sequences via Sanger sequencing. Then, we expressed both constructs using the mammalian HEK293 cells, purified via Ni-NTA affinity column and characterized using SDS-PAGE and western blotting analyses, which confirmed the formation of the constructs (FIG. 16 panels A-B). A Gly3-Alexa647 sortase substrate was synthesized and used to site-specifically label the fusion proteins with AlexaFluor647. Sortase reaction was performed by mixing the construct (50 μM), the Gly3-Alexa647 substrate (500 μM), and sortase (5 μM) for 1 h, at 4° C. The Alexa647-labeled constructs were purified via size-exclusion column chromatography and the installation of the fluorophore was further confirmed by in-gel fluorescence scan of the constructs (FIG. 16 panel B). To characterize the functionality of the constructs, we used Alexa647-labeled mDsg3-mFc to stain the anti-mDsg3 AK23 hybridoma, and the anti-hDsg3 F779 and PVB28 cells. The cells were stained with great affinity and specificity (at 10 nM concentration) (FIG. 16 panel C), suggesting that the patient-derived BCRs on the F779 and PVB28 cells cross-react with mouse Dsg3 protein. Nalm-6 cells, used as control, did not show any binding, confirming the specificity of the construct (FIG. 16 panel C). Similar experiments were performed using the Alexa647-labeled hDsg3-hFc fusion protein, which confirmed that it binds strongly to the F779 and PVB28 cells and also stained the mouse AK23 cells, suggesting the AK23 anti-mouse Dsg3 antibody cross-react with human Dsg3 protein (FIG. 16 panel C). Of note, human Dsg3 is similar to mouse Dsg3 (86% sequence homology)⁶⁸ and functionally rescues the mouse Dsg3 deficiency⁶⁹. To further analyze the constructs, we measured the EC50 values for both mDsg3-mFc and hDsg3-hFc constructs by using different concentrations of the Alexa647-labeled proteins. Results showed that the hDsg3-hFc and mDsg3-mFc bind to F779 cells with an EC50 of 1.8=0.8 nM and 2.3±0.9 nM, respectively (FIG. 16 panels D-E). To further characterize the constructs, we performed ELISA assay by first coating a plate with the AK23 antibody and another plate with P3F3 antibody (anti-human Dsg3)⁶⁴, and then analyzing the binding of the constructs to the coated plates. The results further confirmed that the human and mouse constructs we made specifically bind to both anti-Dsg3 antibodies, but not the control plate (FIG. 16 panel F). P3F3 antibody was made in the Expi293F cells, following the manufacturer's protocol. AK23 antibody was purified from the AK23 hybridoma media using a protein-G affinity column. Following a similar approach, we can generate mouse and human Dsg3-EC1-4, and Dsg3-EC1-5 fused to mouse IgG2a and human IgG1 Fc and will similarly characterize them.

Establishing the Fc-mediated in vitro killing assay. First, we confirmed that the mDsg3-mFc construct, through its Fc portion (IgG2a), binds to the Fcγ receptor of mouse macrophages with low nM affinity (FIG. 16 panel G); EC50 value of 2.3±0.8 nM); control Nalm6 cells remained unstained. To determine whether the fusion protein can result in Fc-mediated killing, F779 target cells, co-cultured with mouse RAW264.7 macrophage cells (Effector: Target ratio of 10:1), were treated with the mDsg3-mFc fusion protein. As low as 1 nM of the construct was able to mediate efficient killing of the F779 target cells (˜60% and ˜80% killing after 24 and 48 hours, respectively, with no killing of Nalm6 control cells; FIG. 17). Similar experiments were performed with PVB28 as target cells (which binds to EC2); results showed a similarly effective killing of the target cells, indicating the treatment can mediate the killing of different autoreactive cells regardless of the targeted epitopes on Dsg3 (FIG. 17). Experiments were repeated with different doses of mDsg3-mFc (1, 10, and 100 nM) to study the effect of the treatment concentration and any potential non-specific killing that can occur at higher treatment concentrations. Results showed a high rate of the killing of targeted cells (˜90% killing), with no non-specific killing of anti-Dsg3-negative Nalm6 cells, at 10 and 100 nM, suggesting the treatment remains highly effective and specific at higher concentration ranges (FIG. 17). We performed similar experiments using RAW264.7 macrophage cells as effector cells and F779 and PVB28 as the target cells, but this time used hDsg3-hFc construct (it is known that mouse Fcγ-receptors (FcγRs) recognizes human Fc; however, human FcR does not recognize mouse Fc)⁶⁵. Results showed highly effective and specific killing, similar to that of mDsg3-mFc experiment discussed above (FIG. 17). Next, we repeated the experiment in a similar setting, but this time used THP1 cells⁷⁰ (a widely used and established human monocyte cell line derived from an AML patient) as effector cells and used hDsg3-hFc construct as the treatment. Results showed an effective killing of target cells with no killing of the control anti-Dsg3-negative Nalm-6 cells (FIG. 17). We performed the killing assay using a range of doses for the treatment (0, 1, 10, and 100 nM of the hDsg3-hFc construct (FIG. 17) and observed an effective killing efficacy for all tested concentrations, suggesting the treatment is effective at low concentrations and remains highly specific at high concentrations.

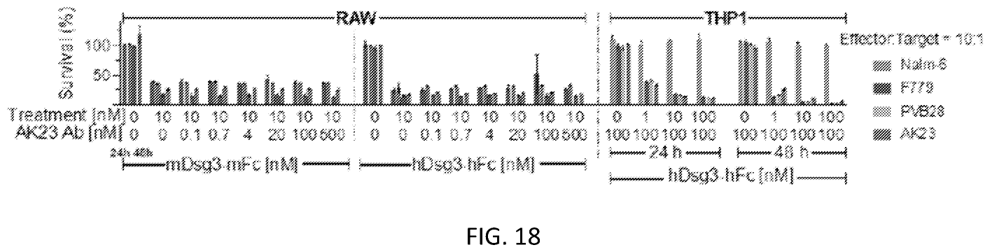

A potential concern is whether the presence of anti-Dsg3 autoantibodies (as occurs in PV patients) can block the treatment from binding to the target cells, and thus decrease or neutralize the treatment efficacy. To study this issue, we first used the well-established AK23 hybridoma cells that release anti-Dsg3 antibodies into the media. The AK23 antibody, recognizes EC1 domain on Dsg3, as discussed above, and thus can potentially neutralize the treatment. Experiment was performed following identical setting as above. Results showed a very effective killing of the AK23 cells (either with Raw cells or THP1 cells as effector cells, and mDsg3-mFc or hDsg3-hFc as treatments), similar to what we observed for the F779 and PVB28 cells (FIG. 17). To perform a more systematic analyses of the effect of the presence of anti-Dsg3 autoantibodies, we repeated the killing assay using AK23 and F779 cells as target cells, Raw cells as the effector cells, 10 nM of mDsg3-Fc or hDsg3-Fc as the treatment and following the same setting as above (Effector: Target ratio of 10:1); however, this time we added a range of known amounts of AK23 antibody to the mixture to assess whether the presence of the anti-Dsg3 antibody may affect the killing efficacy. Strikingly, we observed no change in the killing efficacy in any of the conditions tested (a range of 100 pM to 500 nM AK23 antibody was tested; see FIG. 18; first two columns in each set are 24 h assessments and the next two are 48 h). We repeated the assay using all four different cells (Nalm-6 as control, and then AK23, F779 and PVB28 as target cells), and this time used THP-1 cells as effector cell and used different doses of hDsg3-hFc as the treatment (0, 1, 10 and 100 nM), in the presence of 100 nM of AK23 antibody. Strikingly, and similar to above, there was no blocking observed, and an effective killing was obtained in all conditions tested (FIG. 18). This indicates that the presence of autoantibodies against Dsg3 can have a minimal negative impact on the killing efficacy of the Dsg3-Fc fusion protein.

To further evaluate this potential issue, and to delineate how the presence of polyclonal anti-Dsg3 antibodies can affect the efficacy of the approach, we have made four different patient-derived anti-Dsg3 antibodies P3F3 and P5G6 (bind to Dsg3-EC1), P1F5 (binds to Dsg3-EC 2) and P5D4 (binds to Dsg3-EC4). We have already made the P3F3 antibody and the production of the other three antibodies are underway. We will repeat the killing assays for AK23, F779, and PVB28 cells, in the presence of a mix of these five antibodies, with a range of concentrations, similar to what we have done above (AK23, P3F3, P5D4, P5G6, P1F5). Additionally, we will run an assay where we will use a mix of target cells (AK23, F779, PVB28) in the presence of a mix of anti-Dsg3 antibodies, to closely mimic physiological condition. Without wishing to be bound by theory, the treatment can continue the targeted killing of polyclonal pathogenic B cells in the presence of polyclonal autoantibodies.

Overall, the in vitro data indicate that the Dsg3-Fc constructs can effectively and specifically mediate killing of the anti-Dsg3+ target cells, even in the presence of anti-Dsg3 autoantibodies.

Delineating the efficacy of the human construct (hDsg3-hFc) in in vitro assays using patient PBMCs. Without wishing to be bound by theory, we can delineate the efficacy of the approach in killing pathogenic B cells in presence of polyclonal anti-Dsg3 IgGs present in the patients' blood, and without affecting the normal B cells. We will obtain PBMCs from PV patients (at least n=10) with confirmed autoreactive B cells to Dsg3 protein71. PBMCs obtained from anti-Dsg3 positive patients in different disease phases including: 1) treatment naïve, and 2) relapse of disease, will be cultured in round bottomed 48-well plates (n=10 for each group). The hDsg3-hFc fusion protein (1 nM and 10 nM) will be added to the cells following incubation at 37° C. in a standard cell incubator. No additional phagocytic cells will be added to the mixture, as we expect the patients' own phagocytic cells and NK cells, present in the PBMCs, to be able to kill the targeted anti-Dsg3+ pathogenic B cells. One day and two days post-incubation of the cells and the treatment (hDsg3-hFc), an ELISpot assay will be performed to evaluate for antigen-specific and total IgG B-cell depletion. Results will reveal percentage decrease of the autoreactive anti-Dsg3 IgGs compared to total IgG level (which is expected to be unaffected; therefore, used as control). Analysis will be performed via flow cytometry, and standard live-dead analyses, including on B and T cells, which will act as an internal standard for the assay. In addition to using antibodies to gate for CD3+, CD4+, CD8+ and CD20+ cells, we will use the fluorophore labeled hDsg3 construct (hDsg3-Alexa647) to stain PBMCs. The hDsg3 protein (with no Fc) is already in hand. We made the hDsg3 protein (EC1-3) following a similar protocol as we used to make the described proteins (standard cloning, following expression, purification, and site-specific labeling via sortase to install Alexa647). This will allow gating on anti-Dsg3+ B cells present in the PV patients' PBMCs. Flow cytometry analysis before and after the assay, which includes staining with Alexa647-labeled Dsg3 protein, will reveal how effectively anti-Dsg3 cells were targeted and killed. As a second control, healthy PBMCs will be used with the same settings to further confirm the observed result (here, we expect no change to occur). PV patients' PBMCs will be collected from patients meeting the following inclusion/exclusion criteria: Inclusion criteria: 1) biopsy proven PV; 2) circulating anti-Dsg3 IgG autoantibodies by standard ELISA; 3) normal B lymphocyte counts demonstrated by CD19 and CD20 at the time of PBMC collection. Exclusion criteria: 1) Patients with known blood-born malignancies including B and T cell lymphomas/leukemias. Treatment in the 12-months preceding and at the time of PBMC collection will be documented including any treatment that may affect PBMC level or function. Without wishing to be bound by theory, pathogenic B cells in patients' PBMCs can be efficiently and specifically killed, without affecting the normal B cells.

We already have made >150 mg of mDsg3-EC1-3-mFc, >150 mg of hDsg3-EC1-3-hFc, >200 mg of mDsg3-EC1-3 and >200 mg of hDsg3-EC1-3, which exceed the amount required for the proposed experiments, and can make more as needed. We will similarly make constructs containing murine and human Dsg3-EC1-4-Fc and Dsg3-EC1-5-Fc. If the protein expression yield is low for a particular construct, we can explore different routes to address the issue, including, but not limited to: 1) re-optimize the nucleotide codons used for the expression, 2) use a different signal peptide sequence, and/or 3) use other systems for protein expression instead of HEK293 cells, such as CHO, Expi293F or insect cells.

Regarding the presence of autoantibody and neutralization concern, the results indicate that the presence of autoantibodies, as occurs in the physiological condition in PV patients, will not (or at least not completely) neutralize the treatment. Without wishing to be bound by theory, this is due to the dynamic and reversible nature of the antigen-antibody interaction, which is in contrast to the irreversible process of killing the target cells via effector cells. Furthermore, the clustering that occurs between the effector and target cells, which happens through several Dsg3-Fc molecules bridging the two cells, can be stronger in terms of affinity as compared to autoantibody-interaction with Dsg3-Fc molecules, thereby driving the killing process forward even in the presence of autoantibodies. Importantly, antibodies can bind to different epitopes on Dsg3 protein; many antibodies will therefore have non-overlapping epitopes. Thus, even when autoantibodies are bound to the Dsg3-Fc treatment construct, the complex (antibody bound to Dsg3-Fc) can still bind to anti-Dsg3 BCRs (on the surface of pathogenic B cells) with non-overlapping epitopes and, as such, bridge the effector to the target cells, help to form the cluster between the two cells, and thus mediate the killing of the target cells. Therefore, considering the many potential epitopes on Dsg3 protein and the dynamic nature of the interactions, the likelihood of Dsg3-Fc construct reaching to all pathogenic clones of anti-Dsg3 BCRs remains statistically high. Furthermore, and importantly, we do not see this as a limitation in clinical practice, as the dosing of these constructs can be patient-dependent, potentially based on the level of circulating anti-Dsg3 titer, similar to, but not directly analogous to, drugs like omalizumab (an IgG1k anti-human IgE, used based on the IgE level). Additionally, in real-world setting, the therapeutic can be used concurrently with other drugs that lower anti-Dsg3 titer such as IVIG⁷²⁻⁷⁶. Another approach that is used in the clinic to lower the circulating anti-Dsg3 autoantibodies is plasmapheresis and immunoadsorption, which can reduce the titers of circulating autoantibodies and is usually used for patients with severe pemphigus⁷⁷⁻⁸⁰. Therefore, there are multiple ways to address this (potential) issue and we will continue to carefully assess this as we move forward with the experiments, and as discussed above. Further, reassuringly, monoclonal and polyclonal anti-Dsg3 IgG was found not to have a significant effect on the in vivo killing abilities of CAAR T cells specific for Dsg3-autoantibody producing B cells⁴⁵. Therefore, not only the data, but the efficacy of the anti-Dsg3 CAAR T cells suggest the circulating anti-Dsg3 antibodies will not block the effect of the treatment. Another example (although not directly analogous to the current proposal) is the efficacy of B-cell maturation antigen (BCMA) CAR T cells in multiple myeloma patients, where most patients have high levels of soluble BCMA antigen in the circulation, due to shedding caused by γ-secretase⁸¹; however and in spite of presence of high levels of soluble BCMA in the circulation, patients receiving the BCMA CAR T cells have shown remarkable response suggesting the BCMA CAR T cells can efficiently kill the BCMA+ multiple myeloma cells with specificity and efficacy, even in the presence of soluble BCMA. Overall, these observations indicate the presence of soluble antigen (or autoantibody) will not compromise the activity of effector cells to kill target cells. This can be due to the dynamic nature of interactions, the non-overlapping epitopes, and the clustering effect between the effector and target cells, as discussed above.

To further increase the efficacy of the Fc-mediated approach, we have designed and successfully expressed Dsg3-Fc fused with IL-2, which can engage more cells and enhance the killing. If this increases the efficacy, we can explore other cytokines (e.g., IL-15) as well. For negative control experiments, in addition to cells lacking expression of anti-Dsg3 antibodies, and as an alternative, we have designed and made a plasmid for synthesis of Dsg1-Fc fusion that we can express and use as well.

We can generate similar constructs for Dsg1 protein. Dsg1 and Dsg3 are expressed throughout the squamous layer of mucosa, however, Dsg3 is expressed at a higher level than Dsg1^{1,3,23}. In the skin, although Dsg1 is expressed throughout the epidermis, it is more intensely present in the superficial layers. In contrast to Dsg1, Dsg3 is more intensely expressed in the lower portion of the epidermis, including in the basal and parabasal layers and on a lesser extent in the superficial layers. As a result of these different expression patterns in skin and mucosa: i) Dsg3 behaves as the major pathogenic antigen in patients with the mucosal-dominant type of pemphigus vulgaris, who develop deep erosions in their mucous with no or minimal involvement of the skin (as Dsg1 can compensate for it in the skin); all of the mucosal-dominant PV patients have high levels of anti-Dsg3 IgG autoantibodies^{3,23}; ii) patients with high-levels of anti-Dsg3 and anti-Dsg1 IgG autoantibodies develop mucocutaneous type of pemphigus vulgaris and show deep erosions in mucous and skin; and iii) patients with pemphigus foliaceus (PF) have only high-levels of anti-Dsg1 autoreactive antibodies and only show superficial skin blisters with no mucosal involvement, suggesting the Dsg1 being the major pathogenic autoantigen in PF. We can develop and characterize Dsg3-Fc fusion proteins, which can be used to treat mucosal-dominant type of PV. We can make similar constructs for Dsg1 as well. Without wishing to be bound by theory, patients with mucocutaneous PV can be treated with a mix of Dsg3-Fc and Dsg1-Fc, and patients with PF can be treated with Dsg1-Fc. Additionally, we can generate a Dsg3-Dsg1-Fc knobs-into-holes structure⁸² (Dsg3-knobs+Dsg1-holes), which allows heterodimerization of the CH3 in the Fc part, and thus can target both Dsg1 and Dsg3 autoreactive pathogenic B cells.

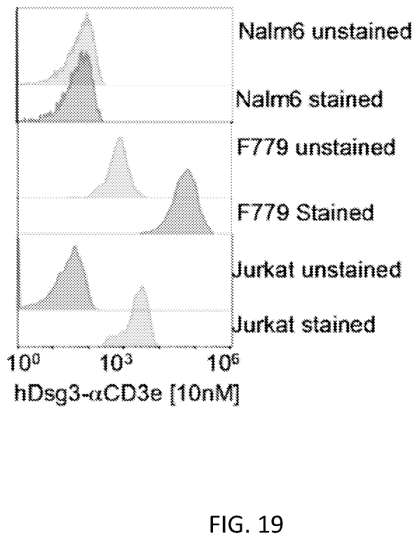

In additional studies, we have made Dsg3-EC1-3 fused to an anti-CD3e single-chain Fv (scFv) antibody fragment, which can direct and engage T cells to target and kill pathogenic autoreactive B cells (FIG. 15 panel B); this approach can bypass potential FcR saturation, and in case a pathogenic B cell is resistant to the killing via the Fc-mediated approach, this T cell mediated approach can provide an alternative. For example, some patients show resistance to rituximab due to genetic polymorphisms, which leads to decreased antibody-dependent cellular cytotoxicity⁸³⁻⁸⁵. The Dsg3-anti-CD3e construct will, without wishing to be bound by theory, establish the molecular clustering and formation of the immunological synapses between the target anti-Dsg3 B cells and the T cells, which will consequently result in the activation of T cells^{52,53} and to specifically kill the targeted anti-Dsg3 B cells, leaving other B cells intact^{52,53,86-88}. Unlike in oncological applications, cytokine release syndrome is not expected in treatment of autoimmune diseases, given the burden of autoreactive B cells is much lower than for malignant clones. Accordingly, the N-to-C Dsg3-anti-CD3e fusion plasmid was designed to contain cDNA for the hDsg3-EC1-3 and the OKT3 anti-CD3e scFv. OKT3 clone has been used in the clinic as the anti-CD3e portion of the bi-specific T cell engagers⁸⁹, and as such we chose to use this clone; of note, CD3e is found on all mature T lymphocytes. The construct was designed to have the Dsg3 on the N-terminus and anti-CD3e scFv on the C-terminus, similar to other bi-specific T cell engagers; furthermore, this structure will ensure that the Dsg3-EC1 and EC2 domains, that are most pathogenic, will be available for recognition by the Dsg3-reactive B-cells. A flexible (G4S)3 spacer/linker was included between Dsg3 and scFv proteins, similar to other bi-specific T cell engagers. The protein was expressed using the HEK293 cells, purified via Ni-NTA beads, and size-exclusion chromatography, and further characterized by SDS-PAGE, anti-FLAG western blot, and flow cytometric analysis. The flow cytometric analyses revealed that the fusion protein binds to established anti-Dsg3 B cells and CD3+ Jurkat cells (human T cells) with high specificity and affinity, but not the control Nalm-6 cells (FIG. 19). We have already made >10 mg of the protein and will make more as needed.

To test the efficacy of Dsg3-Fc fusion proteins to target and mediate the killing of anti-Dsg3 autoreactive B cells in vivo.

In some embodiments, a Dsg3-Fc construct can mediate targeted depletion of the circulating anti-Dsg3⁺ autoreactive B cells. In some embodiments, the presence of autoantibodies does not affect the targeted depletion.

To test the in vivo efficacy of the Dsg3-Fc fusion proteins for specific targeting and depletion of circulating anti-Dsg3+ B cells, anti-DSG expressing PVB-28 malignant cells were engrafted in immuno-deficient NOD-scid gamma (NSG) mice. NSG mice lack mature T cells, B cells, and NK cells, show reduced dendritic cell function, and defective macrophage activity, and as such are widely used for the engraftment of a wide range of human cells for preclinical studies. Accordingly, the NSG mice were intravenously injected via tail vein (i.v.) with luciferase-positive PVB28 cells (1×10⁶) on day 0. Mice were pretreated with 16 mg/kg IVIG (intraperitoneal (i.p.) injection) on days −3, −2 and −1 prior to PBV28 cell administration, to block nonspecific killing via FcγR (i.e., to enhance engraftment of the PVB28 cells). mDsg3-mFc treatment was administered to the treatment group, injected i.p. 3 times a week for two weeks and then one per week, starting on day 4. The control group received an isotype IgG2a. The autoantibodies group received a mix of AK23, AK19 and AK18 (150 μg; anti-Dsg antibodies from hybridomas) twice per week for two weeks, starting a day prior to initiation of treatment (i.e., on day 3) all mice (treatment and control). Disease progression was monitored by bioluminescence imaging (BLI).

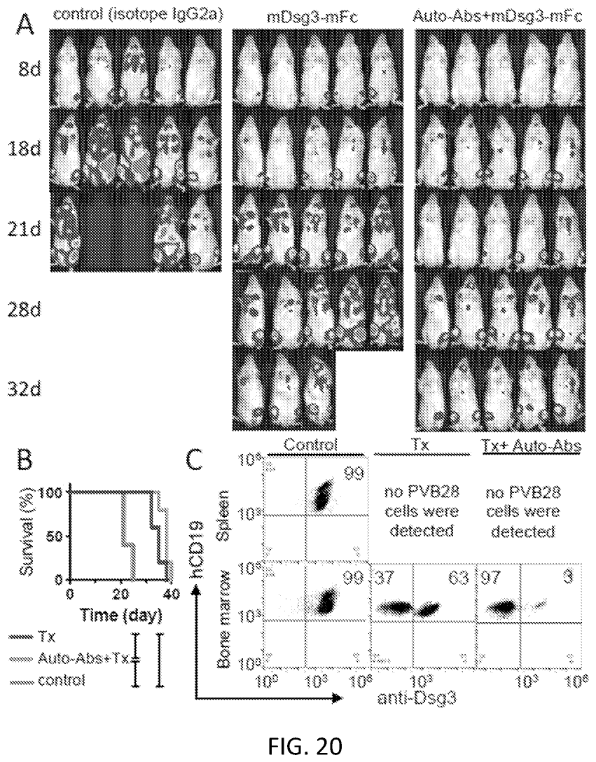

FIG. 20 panel A shows BLI imaging showing a slower signal enhancement in treatment-recipient mice, even in the presence of antibodies. FIG. 20 panel B shows the survival rate is significantly higher in both treatment-recipient groups compared to the control group (P value=0.0023). Also, the median of survival for autoantibodies+treatment (Auto-Abs+Tx) group was 38 days, which is higher than the treatment group (median survival=35 days; P value=0.048). FIG. 20 panel C shows flow cytometric analysis at the end point and demonstrated complete depletion of PBV28 cells in bone marrow of the treatment-recipient mice (data representative of n=5 for each group).

Animal studies in PV mouse models. The efficacy of the approach will be tested in two PV mouse models:

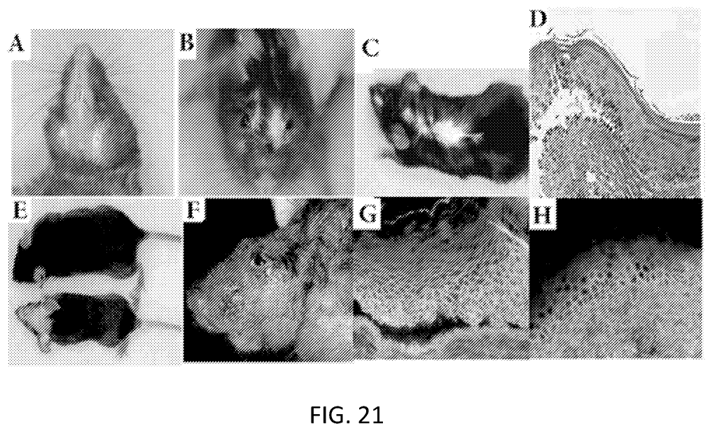

Passive Transfer Model. In this model, we can develop a passive monoclonal pemphigus mouse disease model by injecting AK23 antibody⁹¹, which is known to result in the development of blisters in both skin and mucosa²⁶, this mouse model recapitulates the major features described in PV patients²⁶. As such, an experiment with identical setting as explained above can be performed, with the only difference that animals will be pre-injected with 200 μg AK23 antibody, 3 times per week starting 2 days prior to the initiation of treatment, and for the duration of treatment. This can ensure that the mice will have a constantly high amount of anti-Dsg3 autoantibody in the circulation for the duration of treatment. A purpose of this experiment is to delineate the effect of the presence of pathogenic anti-Dsg3 autoantibodies in the circulation on the efficacy of the treatment. The read-out will be the BLI analyses and survival, same as above. Once established, we can repeat the same experiment, however, this time we can inject polyclonal pathogenic B cells (mix of F779 and PVB28) and polyclonal anti-Dsg3 autoantibodies (mix of AK23, P1F5, P3F3, P5G6, and P5D4 antibodies; 200 μg total antibody per injection, 40 μg of each). This can show that the treatment will remain effective even in the presence of polyclonal autoantibodies and polyclonal pathogenic Dsg3-reactive B cells. This setting can resemble the physiological condition in PV patients. We can test for establishment of the disease by analyzing serum anti-Dsg3 titers (by standard ELISA), and skin blisters (assessed by blinded investigators), three times per week post injection of pathogenic antibodies. Without wishing to be bound by theory, the mice can exhibit hair loss, weight loss, and have blistering in hair follicles and oral mucosa along with autoantibodies binding to epidermal keratinocytes (FIG. 21 panels A-D) 91.48 BLI imaging can be used to monitor the killing efficacy of the treatment, similar to above. Two sets of control can be used: mice that will not receive the treatment (negative control), and mice that will receive the treatment but not the autoantibodies (positive control; we have already showed treatment results in near full response in this setting).

Active Transfer Model. The passive disease model has advantages; however, treatment should overcome the fast-proliferating malignant cells, which may not be the case in PV patients. Therefore, as a second model, we can use an active PV immune model that closely mimics the clinical phenotype of human disease, has lower frequency of autoreactive Dsg3-specific B cells that resembles what happens clinically, and finally has a high serum polyclonal anti-Dsg3 autoantibody level. In this model, we can develop an active polyclonal pemphigus mouse disease model by first immunizing Dsg3−/− mice (JAX-002911) with the recombinant extracellular domain of mouse Dsg3 (already produced and in hand), followed by the transfer of splenocytes into RAG2−/− mice⁹⁵⁻⁹⁷. Transferred cells will contain B cells (and T cells), which can include pathogenic Dsg3-reactive B cells. Similar to observations made in the literature^{95,96,98}, without wishing to be bound by theory, animals can have stunted growth (FIG. 21 panel E) and develop mucocutaneous erosions with suprabasal acantholysis and blisters (FIG. 21 panels F-H). To assess the clinical index, using ELISA we can compare autoantibody levels to physiological levels in PV patients (300-400 RU/ml)⁴⁸. When the Dsg3 reactive antibodies equal to or exceed that level, which can happen in 3-4 weeks^{42,48}, treatment can be initiated using the same setting as discussed above (200 μg of mDsg3-mFc per mouse, 3 times per week for two weeks). Adoptive transfer of cells from Dsg3-immunized Dsg3+/− animals can be used as a control98 (at least n=10 mice per group).

Serum anti-Dsg3 titers measured at days 5 and 14 post-initiation of treatments, blinded investigation of skin erosions, immunofluorescence of mucosa samples to detect IgG deposition, histological assessment of mucosal barriers, and the quantitation of the histopathology can be performed to further assess the efficacy of the approach. B cell counts can be performed as well to ensure they remain within normal levels during the treatment. Without wishing to be bound by theory, the treatments can result in clinical and histological resolution of the disease, the intended outcome.

To further ensure safety of the treatment and that there will be no off-target cytotoxic interactions, we can perform toxicology screening using primary human cells and high-throughput membrane proteome arrays. Without wishing to be bound by theory, BCR-engagement-induced endocytosis can occur with our therapeutics, e.g., with “antigens” such as the Dsg3-Fc that engage the anti-Dsg3 BCR alone without co-engagement of other receptors, thus inducing a low rate of endocytosis⁹⁹. Additionally, in vitro and in vivo data support that engagement of Dsg3-Fc with anti-Dsg3 BCR result in targeted, almost complete killing of the Dsg3-recognizing autoreactive B cells; thus, based on current evidence, if anti-BCR engagement-induced endocytosis and degradation occur, it cannot have clinical significance. Additionally, the antibodies produced by Dsg3-recognizing autoreactive B cells (the target of our therapeutic), by definition, inherently target our therapeutics (Dsg3-Fc); thus, we are not as concerned about the generation of “neutralizing antibodies” as seen in other therapeutics such as Rituximab or anti-TNF-alphas that decrease their efficacy, and as discussed in detail above; especially, see our discussion on how the non-overlapping epitopes can positively contribute in overcoming the neutralization issue).

In the active disease model, anti-Dsg3 levels can be measured by ELISA once per week to test long-lasting remission. Mice can be tested by a blinded investigator to check for skin lesions to ensure no disease relapse. The Fc-mediated approach can show relapse, as with rituximab, but without wishing to be bound by theory, they can respond with retreatment. Interestingly, some studies indicate that majority of patients treated with multiple cycles of rituximab over several years show long-term remission¹⁰⁰. However, because rituximab depletes the patients' whole B cell pool, and repeated infusion can result in delayed repopulation of B cells, it carries risk of infection and would obliterate the humoral response to new infections or vaccinations. The targeted nature of the Dsg3-Fc approach developed here allows it to be used as a continuous treatment in multiple cycles without the fear of any major side-effects. For example, after the first course of treatment is over, and when patients are in remission, they can be re-injected once in three months (or even more frequently) for a year or even longer. In a mechanism analogous to serial and multi-cycle rituximab treatment, this can ensure that all Dsg3 pathogenic B cells will be depleted. As the Dsg3-Fc approach does not impact normal B cell pool, without wishing to be bound by theory, multiple use of it on patients over several months should face no major issues, and thus would allow long-term remission; this is an advantage over anti-CD20 biologics. Thus, complete, targeted depletion of autoreactive B cells, as proposed here, can result in prolonged to permanent remission.

We can study the dose-dependency of the treatment and how its effectiveness correlates with the presence of anti-Dsg3 autoantibody titers (via ELISA). With the wide-spread use of biologics, anti-drug antibodies have been reported for multiple agents ranging from anti-TNFαs¹⁰¹ to rituximab^{102,103} Major immune adverse events, such as immune-complex deposition, have been theorized¹⁰⁴, but have not actualized in clinical practice. In cases where the biologic binds to the high circulating antibodies, such as in the case of omalizumab, dosing patients with high IgE level did not result in immune adverse events¹⁰⁵. Similarly, presence of anti-Dsg3 antibody in serum will not result in any major immune adverse events; this can be monitored, as discussed herein. For the active PV model experiments, and as a positive control, we can explore using anti-CD20 IgG (clone SA271G2; 250 μg per animal) as it has been shown to effectively deplete B cells in mice¹⁰⁶.

For the passive transfer PV model (model 1), as an alternative to injecting pathogenic antibodies, we can inject only luciferase-positive AK23 cells into NSG mice⁴⁵, as the cells will produce pathogenic anti-Dsg3 antibody, which is known to cause disease⁴⁵. The readout will be BLI, same as discussed herein.

To verify that the Dsg3-Fc treatment will not globally suppress the immune system, the animals with active PV (model 2), post-treatment with Dsg3-Fc, can be infected with active LCMV-C113 (2×10⁶ PFU, i.v.). C57BL/6 will be used as a positive control. Viral load in liver and blood of infected mice can be compared 30 days post-injection using plaque assay¹⁰⁷⁻¹⁰⁹. Alternatively, for acute infection post-treatment, mice can be challenged orally with Listeria monocytogenes (10⁴-10⁵ CFU) or intraperitoneally with LCMV Armstrong (2×10⁵ PFU)¹¹⁰⁻¹¹². Microbial load in liver and spleen can be checked 10 days post-infection and compared with infection-only mice. In both experiments, without wishing to be bound by theory, there will not be any difference in the viral load.

The BCR-T cell engager approach (Dsg3-fusion to anti-CD3) can be an alternative approach, as discussed herein. To accomplish this, we have made the Dsg3-anti-CD3e fusion protein and characterized it, as discussed herein. A clinical issue with molecules of this type, due to their small size, is their sometimes less than optimal in vivo pharmacokinetic properties¹¹³ (short half-life, t_{1/2}˜0.6 h). To resolve this issue, we can develop a bispecific antibody construct with Dsg3 ectodomain on one arm and the anti-CD3 scFv on the other arm. We can use the Knobs-into-Holes approach to attach two different targeting agents into one antibody structure while using the LALA mutation to ensure no macrophage recruitment¹¹⁴. This structure can ensure high pharmacokinetic properties due to the Fc region as well as better expression, stability, and clinical applicability.

For the performed in vivo experiments that are discussed herein, we have used both male and female mice and no difference was observed between sexes in either control or treatment groups (for example, a total of n=27 NSG mice have been used for the in vivo studies, with 8 male and 19 female, that were randomly assigned to each experiment); regardless of sex, control mice showed increased BLI signal and had to be euthanized by day ˜30, whereas treatment mice showed ˜100% antigen-loss at the end point, or showed full response and continue to be healthy with no detectable BLI signal (for the PVB28 experiment) (FIG. 20A-C). We will continue to use both sexes in experiments and will monitor any differences we may observe, especially for the active PV model.

Methods. Cloning, protein production and characterization, western blot, flow cytometric and ELISA analyses, and animal BLI imaging were performed following standard lab approaches. In vitro and in vivo efficacy assessment experiments were performed as explained above. For details on developing the PV passive and active models see the vertebrate animal section.

### References Cited in this Example

1. Mahoney M G, Wang Z, Rothenberger K, Koch P J, Amagai M, Stanley J R. Explanations for the clinical and microscopic localization of lesions in pemphigus foliaceus and vulgaris. J Clin Invest. 1999 Feb. 15; 103 (4): 461-468.
2. Kasperkiewicz M, Ellebrecht C T, Takahashi H, Yamagami J, Zillikens D, Payne A S, Amagai M. Pemphigus. Nat Rev Dis Primers. 2017 May 11; 3:17026. PMCID: PMC5901732
3. Schmidt E, Kasperkiewicz M, Joly P. Pemphigus. The Lancet. 2019 September; 394 (10201): 882-894.
4. Murrell D F, Peña S, Joly P, Marinovic B, Hashimoto T, Diaz L A, Sinha A A, Payne A S, Daneshpazhooh M, Eming R, Jonkman M F, Mimouni D, Borradori L, Kim S-C, Yamagami J, Lehman J S, Saleh M A, Culton D A, Czernik A, Zone J J, Fivenson D, Ujiie H, Wozniak K, Akman-Karakaş A, Bernard P, Korman N J, Caux F, Drenovska K, Prost-Squarcioni C, Vassileva S, Feldman R J, Cardones A R, Bauer J, Ioannides D, Jedlickova H, Palisson F, Patsatsi A, Uzun S, Yayli S, Zillikens D, Amagai M, Hertl M, Schmidt E, Aoki V, Grando S A, Shimizu H, Baum S, Cianchini G, Feliciani C, Iranzo P, Mascaró J M, Kowalewski C, Hall R, Groves R, Harman K E, Marinkovich M P, Maverakis E, Werth V P. Diagnosis and management of pemphigus: Recommendations of an international panel of experts. J Am Acad Dermatol. 2020 March; 82 (3): 575-585.e1. PMCID: PMC7313440
5. Koch P J, Walsh M J, Schmelz M, Goldschmidt M D, Zimbelmann R, Franke W W. Identification of desmoglein, a constitutive desmosomal glycoprotein, as a member of the cadherin family of cell adhesion molecules. Eur J Cell Biol. 1990 October; 53 (1): 1-12. PMID:
6. Amagai M, Klaus-Kovtun V, Stanley J R. Autoantibodies against a novel epithelial cadherin in pemphigus vulgaris, a disease of cell adhesion. Cell. 1991 Nov. 29; 67 (5): 869-877. PMID: 1720352
7. Sajda T, Sinha A A. Autoantibody Signaling in Pemphigus Vulgaris: Development of an Integrated Model. Front Immunol. 2018; 9:692. PMCID: PMC5932349
8. Mascaró J M, España A, Liu Z, Ding X, Swartz S J, Fairley J A, Diaz L A. Mechanisms of acantholysis in pemphigus vulgaris: role of IgG valence. Clin Immunol Immunopathol. 1997 October; 85 (1): 90-96. PMID: 9325074
9. Furue M, Kadono T. Pemphigus, a pathomechanism of acantholysis. Australas J Dermatol. 2017 August; 58 (3): 171-173. PMID: 28211055
10. Murrell D F, Dick S, Ahmed A R, Amagai M, Barnadas M A, Borradori L, Bystryn J-C, Cianchini G, Diaz L, Fivenson D, Hall R, Harman K E, Hashimoto T, Hertl M, Hunzelmann N, Iranzo P, Joly P, Jonkman M F, Kitajima Y, Korman N J, Martin L K, Mimouni D, Pandya A G, Payne A S, Rubenstein D, Shimizu H, Sinha A A, Sirois D, Zillikens D, Werth V P. Consensus statement on definitions of disease, end points, and therapeutic response for pemphigus. J Am Acad Dermatol. 2008 June; 58 (6): 1043-1046. PMCID: PMC2829665
11. Joly P, Maho-Vaillant M, Prost-Squarcioni C, Hebert V, Houivet E, Calbo S, Caillot F, Golinski M L, Labeille B, Picard-Dahan C, Paul C, Richard M-A, Bouaziz J D, Duvert-Lehembre S, Bernard P, Caux F, Alexandre M, Ingen-Housz-Oro S, Vabres P, Delaporte E, Quereux G, Dupuy A, Debarbieux S, Avenel-Audran M, D'Incan M, Bedane C, Bénéton N, Jullien D, Dupin N, Misery L, Machet L, Beylot-Barry M, Dereure O, Sassolas B, Vermeulin T, Benichou J, Musette P, French study group on autoimmune bullous skin diseases. First-line rituximab combined with short-term prednisone versus prednisone alone for the treatment of pemphigus (Ritux 3): a prospective, multicentre, parallel-group, open-label randomised trial. Lancet. 2017 May 20; 389 (10083): 2031-2040. PMID: 28342637
12. Committee for Guidelines for the Management of Pemphigus Disease, Amagai M, Tanikawa A, Shimizu T, Hashimoto T, Ikeda S, Kurosawa M, Niizeki H, Aoyama Y, Iwatsuki K, Kitajima Y. Japanese guidelines for the management of pemphigus. J Dermatol. 2014 June; 41 (6): 471-486. PMID: 24909210
13. Hertl M, Jedlickova H, Karpati S, Marinovic B, Uzun S, Yayli S, Mimouni D, Borradori L, Feliciani C, Ioannides D, Joly P, Kowalewski C, Zambruno G, Zillikens D, Jonkman M F. Pemphigus. S2 Guideline for diagnosis and treatment—guided by the European Dermatology Forum (EDF) in cooperation with the European Academy of Dermatology and Venereology (EADV). J Eur Acad Dermatol Venereol. 2015 March; 29 (3): 405-414. PMID: 25338479
14. Atzmony L, Hodak E, Gdalevich M, Rosenbaum O, Mimouni D. Treatment of pemphigus vulgaris and pemphigus foliaceus: a systematic review and meta-analysis. Am J Clin Dermatol. 2014 December; 15 (6): 503-515. PMID: 25403548
15. Werth V P. Treatment of pemphigus vulgaris with brief, high-dose intravenous glucocorticoids. Arch Dermatol. 1996 December; 132 (12): 1435-1439. PMID: 8961871
16. Mignogna M D, Lo Muzio L, Ruoppo E, Fedele S, Lo Russo L, Bucci E. High-dose intravenous “pulse” methylprednisone in the treatment of severe oropharyngeal pemphigus: a pilot study. J Oral Pathol Med. 2002 July; 31 (6): 339-344. PMID: 12201245
17. Almugairen N, Hospital V, Bedane C, Duvert-Lehembre S, Picard D, Tronquoy A-F, Houivet E, D'incan M, Joly P. Assessment of the rate of long-term complete remission off therapy in patients with pemphigus treated with different regimens including medium- and high-dose corticosteroids. J Am Acad Dermatol. 2013 October; 69 (4): 583-588. PMID: 23850258
18. Beissert S, Werfel T, Frieling U, Böhm M, Sticherling M, Stadler R, Zillikens D, Rzany B, Hunzelmann N, Meurer M, Gollnick H, Ruzicka T, Pillekamp H, Junghans V, Luger T A. A comparison of oral methylprednisolone plus azathioprine or mycophenolate mofetil for the treatment of pemphigus. Arch Dermatol. 2006 November; 142 (11): 1447-1454. PMID: 17116835
19. Chams-Davatchi C, Esmaili N, Daneshpazhooh M, Valikhani M, Balighi K, Hallaji Z, Barzegari M, Akhyani M, Ghodsi S Z, Seirafi H, Nazemi M-J T, Mortazavi H, Mirshams-Shahshahani M. Randomized controlled open-label trial of four treatment regimens for pemphigus vulgaris. J Am Acad Dermatol. 2007 October; 57 (4): 622-628. PMID: 17583373
20. Beissert S, Mimouni D, Kanwar A J, Solomons N, Kalia V, Anhalt G J. Treating pemphigus vulgaris with prednisone and mycophenolate mofetil: a multicenter, randomized, placebo-controlled trial. J Invest Dermatol. 2010 August; 130 (8): 2041-2048. PMID: 20410913
21. Ioannides D, Apalla Z, Lazaridou E, Rigopoulos D. Evaluation of mycophenolate mofetil as a steroid-sparing agent in pemphigus: a randomized, prospective study. J Eur Acad Dermatol Venereol. 2012 July; 26 (7): 855-860. PMID: 21752101
22. Chams-Davatchi C, Mortazavizadeh A, Daneshpazhooh M, Davatchi F, Balighi K, Esmaili N, Akhyani M, Hallaji Z, Seirafi H, Mortazavi H. Randomized double blind trial of prednisolone and azathioprine, vs. prednisolone and placebo, in the treatment of pemphigus vulgaris. J Eur Acad Dermatol Venereol. 2013 October; 27 (10): 1285-1292. PMID:
23. Amagai M, Tsunoda K, Zillikens D, Nagai T, Nishikawa T. The clinical phenotype of pemphigus is defined by the anti-desmoglein autoantibody profile. Journal of the American Academy of Dermatology. 1999 February; 40 (2): 167-170.
24. Anhalt G J, Labib R S, Voorhees J J, Beals T F, Diaz L A. Induction of pemphigus in neonatal mice by passive transfer of IgG from patients with the disease. N Engl J Med. 1982 May 20; 306 (20): 1189-1196. PMID: 7040962
25. Rock B, Labib R S, Diaz L A. Monovalent Fab′ immunoglobulin fragments from endemic pemphigus foliaceus autoantibodies reproduce the human disease in neonatal Balb/c mice. J Clin Invest. 1990 January; 85 (1): 296-299. PMCID: PMC296418
26. Schulze K, Galichet A, Sayar B S, Scothern A, Howald D, Zymann H, Siffert M, Zenhäusern D, Bolli R, Koch P J, Garrod D, Suter M M, Müller E J. An Adult Passive Transfer Mouse Model to Study Desmoglein 3 Signaling in Pemphigus Vulgaris. Journal of Investigative Dermatology. 2012 February; 132 (2): 346-355.
27. Joly P, Mouquet H, Roujeau J-C, D'Incan M, Gilbert D, Jacquot S, Gougeon M-L, Bedane C, Muller R, Dreno B, Doutre M-S, Delaporte E, Pauwels C, Franck N, Caux F, Picard C, Tancrede-Bohin E, Bernard P, Tron F, Hertl M, Musette P. A single cycle of rituximab for the treatment of severe pemphigus. N Engl J Med. 2007 Aug. 9; 357 (6): 545-552. PMID: 17687130
28. Feldman R J, Ahmed A R. Relevance of rituximab therapy in pemphigus vulgaris: analysis of current data and the immunologic basis for its observed responses. Expert Rev Clin Immunol. 2011 July; 7 (4): 529-541. PMID: 21790294
29. Wang H-H, Liu C-W, Li Y-C, Huang Y-C. Efficacy of rituximab for pemphigus: a systematic review and meta-analysis of different regimens. Acta Derm Venereol. 2015 November; 95 (8): 928-932. PMID: 25881672
30. Lunardon L, Tsai K J, Propert K J, Fett N, Stanley J R, Werth V P, Tsai D E, Payne A S. Adjuvant rituximab therapy of pemphigus: a single-center experience with 31 patients. Arch Dermatol. 2012 September; 148 (9): 1031-1036. PMCID: PMC3658473
31. Kasperkiewicz M, Eming R, Behzad M, Hunzelmann N, Meurer M, Schulze-Koops H, von Wussow P, Hertl M, Zillikens D, Freivogel K, Dörner T, Schmidt E. Efficacy and safety of rituximab in pemphigus: experience of the German Registry of Autoimmune Diseases. J Dtsch Dermatol Ges. 2012 October; 10 (10): 727-732. PMID: 22577946
32. Tony H-P, Burmester G, Schulze-Koops H, Grunke M, Henes J, Kötter I, Haas J, Unger L, Lovric S, Haubitz M, Fischer-Betz R, Chehab G, Rubbert-Roth A, Specker C, Weinerth J, Holle J, Müller-Ladner U, König R, Fiehn C, Burgwinkel P, Budde K, Sörensen H, Meurer M, Aringer M, Kieseier B, Erfurt-Berge C, Sticherling M, Veelken R, Ziemann U, Strutz F, von Wussow P, Meier F M P, Hunzelmann N, Schmidt E, Bergner R, Schwarting A, Eming R, Hertl M, Stadler R, Schwarz-Eywill M, Wassenberg S, Fleck M, Metzler C, Zettl U, Westphal J, Heitmann S, Herzog A L, Wiendl H, Jakob W, Schmidt E, Freivogel K, Dörner T, GRAID investigators. Safety and clinical outcomes of rituximab therapy in patients with different autoimmune diseases: experience from a national registry (GRAID). Arthritis Res Ther. 2011 May 13; 13 (3): R75. PMCID: PMC3218885
33. Kushner C J, Wang S, Tovanabutra N, Tsai D E, Werth V P, Payne A S. Factors Associated With Complete Remission After Rituximab Therapy for Pemphigus. JAMA Dermatol. 2019 Dec. 1; 155 (12): 1404.
34. Mahmoudi H, Balighi K, Tavakolpour S, Daneshpazhooh M. Unexpected worsening of pemphigus vulgaris after rituximab: A report of three cases. Int Immunopharmacol. 2019 June; 71:40-42. PMID: 30877872
35. Goebeler M, Bata-Csörgö Z, De Simone C, Didona B, Remenyik E, Reznichenko N, Stoevesandt J, Ward E S, Parys W, de Haard H, Dupuy P, Verheesen P, Schmidt E, Joly P, ARGX-113-1701 Investigator Study Group. Treatment of pemphigus vulgaris and foliaceus with efgartigimod, a neonatal Fc receptor inhibitor: a phase II multicentre, open-label feasibility trial. Br J Dermatol. 2021 Oct. 5; PMID: 34608631
36. Werth V P, Culton D A, Concha J S S, Graydon J S, Blumberg L J, Okawa J, Pyzik M, Blumberg R S, Hall R P. Safety, Tolerability, and Activity of ALXN1830 Targeting the Neonatal Fc Receptor in Chronic Pemphigus. J Invest Dermatol. 2021 December; 141 (12): 2858-2865.e4. PMID: 34126109
37. Izumi K, Bieber K, Ludwig R J. Current Clinical Trials in Pemphigus and Pemphigoid. Front Immunol. 2019; 10:978. PMCID: PMC6509547
38. Goodale E C, White S D, Bizikova P, Borjesson D, Murrell D F, Bisconte A, Francesco M, Hill R J, Masjedizadeh M, Nunn P, Gourlay S G, Jordan T J M, Emery C B, Outerbridge C A. Open trial of Bruton's tyrosine kinase inhibitor (PRN1008) in the treatment of canine pemphigus foliaceus. Vet Dermatol. 2020 October; 31 (5): 410-e110. PMID: 32803903
39. Goodale E C, Varjonen K E, Outerbridge C A, Bizikova P, Borjesson D, Murrell D F, Bisconte A, Francesco M, Hill R J, Masjedizadeh M, Nunn P, Gourlay S G, White S D. Efficacy of a Bruton's Tyrosine Kinase Inhibitor (PRN-473) in the treatment of canine pemphigus foliaceus. Vet Dermatol. 2020 August; 31 (4): 291-e71. PMID: 31899567
40. Peter H-H, Ochs H D, Cunningham-Rundles C, Vinh D C, Kiessling P, Greve B, Jolles S. Targeting FcRn for immunomodulation: Benefits, risks, and practical considerations. J Allergy Clin Immunol. 2020 September; 146 (3): 479-491.e5. PMCID: PMC7471860
41. Hammers C M, Chen J, Lin C, Kacir S, Siegel D L, Payne A S, Stanley J R. Persistence of anti-desmoglein 3 IgG (+) B-cell clones in pemphigus patients over years. J Invest Dermatol. 2015 March; 135 (3): 742-749. PMCID: PMC4294994
42. Lee J, Lundgren D K, Mao X, Manfredo-Vieira S, Nunez-Cruz S, Williams E F, Assenmacher C-A, Radaelli E, Oh S, Wang B, Ellebrecht C T, Fraietta J A, Milone M C, Payne A S. Antigen-specific B cell depletion for precision therapy of mucosal pemphigus vulgaris. J Clin Invest. 2020 Dec. 1; 130 (12): 6317-6324. PMCID: PMC7685721
43. Colliou N, Picard D, Caillot F, Calbo S, Le Corre S, Lim A, Lemercier B, Le Mauff B, Maho-Vaillant M, Jacquot S, Bedane C, Bernard P, Caux F, Prost C, Delaporte E, Doutre M-S, Dreno B, Franck N, Ingen-Housz-Oro S, Chosidow O, Pauwels C, Picard C, Roujeau J-C, Sigal M, Tancrede-Bohin E, Templier I, Eming R, Hertl M, D'Incan M, Joly P, Musette P. Long-term remissions of severe pemphigus after rituximab therapy are associated with prolonged failure of desmoglein B cell response. Sci Transl Med. 2013 Mar. 6; 5 (175): 175ra30. PMID: 23467561
44. Esensten J H, Muller Y D, Bluestone J A, Tang Q. Regulatory T-cell therapy for autoimmune and autoinflammatory diseases: The next frontier. J Allergy Clin Immunol. 2018 December; 142 (6): 1710-1718. PMID: 30367909
45. Ellebrecht C T, Bhoj V G, Nace A, Choi E J, Mao X, Cho M J, Di Zenzo G, Lanzavecchia A, Seykora J T, Cotsarelis G, Milone M C, Payne A S. Reengineering chimeric antigen receptor T cells for targeted therapy of autoimmune disease. Science. 2016 Jul. 8; 353 (6295): 179-184. PMCID: PMC5343513
46. Kim A R, Han D, Choi J Y, Seok J, Kim S-E, Seo S-H, Takahashi H, Amagai M, Park S-H, Kim S-C, Shin E-C, Kim J H. Targeting inducible costimulator expressed on CXCR5+PD-1+ TH cells suppresses the progression of pemphigus vulgaris. J Allergy Clin Immunol. 2020 November; 146 (5): 1070-1079.e8. PMID: 32311391
47. Hashimoto K, Shafran K M, Webber P S, Lazarus G S, Singer K H. Anti-cell surface pemphigus autoantibody stimulates plasminogen activator activity of human epidermal cells. A mechanism for the loss of epidermal cohesion and blister formation. J Exp Med. 1983 Jan. 1; 157 (1): 259-272. PMCID: PMC2186905
48. Schmidt E, Dahnrich C, Rosemann A, Probst C, Komorowski L, Saschenbrecker S, Schlumberger W, Stocker W, Hashimoto T, Brocker E-B, Recke A, Rose C, Zillikens D. Novel ELISA systems for antibodies to desmoglein 1 and 3: correlation of disease activity with serum autoantibody levels in individual pemphigus patients. Exp Dermatol. 2010 May; 19 (5): 458-463. PMID: 20163452
49. Yuan H, Zhou S, Liu Z, Cong W, Fei X, Zeng W, Zhu H, Xu R, Wang Y, Zheng J, Pan M. Pivotal Role of Lesional and Perilesional T/B Lymphocytes in Pemphigus Pathogenesis. J Invest Dermatol. 2017 November; 137 (11): 2362-2370. PMID: 28647348
50. Albers L N, Liu Y, Bo N, Swerlick R A, Feldman R J. Developing biomarkers for predicting clinical relapse in pemphigus patients treated with rituximab. J Am Acad Dermatol. 2017 December; 77 (6): 1074-1082. PMID: 28927663
51. Mouquet H, Musette P, Gougeon M-L, Jacquot S, Lemercier B, Lim A, Gilbert D, Dutot I, Roujeau J C, D'Incan M, Bedane C, Tron F, Joly P. B-cell depletion immunotherapy in pemphigus: effects on cellular and humoral immune responses. J Invest Dermatol. 2008 December; 128 (12): 2859-2869. PMID: 18563177
52. Huehls A M, Coupet T A, Sentman C L. Bispecific T-cell engagers for cancer immunotherapy. Immunol Cell Biol. 2015 March; 93 (3): 290-296. PMCID: PMC4445461
53. Yuraszeck T, Kasichayanula S, Benjamin J E. Translation and Clinical Development of Bispecific T-cell Engaging Antibodies for Cancer Treatment. Clin Pharmacol Ther. 2017 May; 101 (5): 634-645. PMCID: PMC5763312
54. Mazmanian S K, Liu G, Ton-That H, Schneewind O. Staphylococcus aureus Sortase, an Enzyme that Anchors Surface Proteins to the Cell Wall. Science. 1999 Jul. 30; 285 (5428): 760-763.
55. Dorr B M, Ham H O, An C, Chaikof E L, Liu D R. Reprogramming the specificity of sortase enzymes. Proc Natl Acad Sci USA. 2014 Sep. 16; 111 (37): 13343-13348.
56. Rashidian M, Keliher E J, Bilate A M, Duarte J N, Wojtkiewicz G R, Jacobsen J T, Cragnolini J, Swee L K, Victora G D, Weissleder R, Ploegh H L. Noninvasive imaging of immune responses. Proc Natl Acad Sci USA. 2015 May 12; 112 (19): 6146-6151. PMCID: PMC4434737
57. Rashidian M, Keliher E, Dougan M, Juras P K, Cavallari M, Wojtkiewicz G R, Jacobsen J, Edens J G, Tas J M G, Victora G, Weissleder R, Ploegh H. The use of (18) F-2-fluorodeoxyglucose (FDG) to label antibody fragments for immuno-PET of pancreatic cancer. ACS Cent Sci. 2015 Jun. 24; 1 (3): 142-147. PMCID: PMC4778250
58. Rashidian M, Wang L, Edens J G, Jacobsen J T, Hossain I, Wang Q, Victora G D, Vasdev N, Ploegh H, Liang S H. Enzyme-Mediated Modification of Single-Domain Antibodies for Imaging Modalities with Different Characteristics. Angew Chem Int Ed Engl. 2016 Jan. 11; 55 (2): 528-533. PMCID: PMC4718774
59. Ingram J R, Dougan M, Rashidian M, Knoll M, Keliher E J, Garrett S, Garforth S, Blomberg O S, Espinosa C, Bhan A, Almo S C, Weissleder R, Lodish H, Dougan S K, Ploegh H L. PD-L1 is an activation-independent marker of brown adipocytes. Nat Commun. 2017 Sep. 21; 8 (1): 647. PMCID: PMC5608754
60. Rashidian M, Ingram J R, Dougan M, Dongre A, Whang K A, LeGall C, Cragnolini J J, Bierie B, Gostissa M, Gorman J, Grotenbreg G M, Bhan A, Weinberg R A, Ploegh H L. Predicting the response to CTLA-4 blockade by longitudinal noninvasive monitoring of CD8 T cells. J Exp Med. 2017 Aug. 7; 214 (8): 2243-2255. PMCID: PMC5551571
61. Cong M, Tavakolpour S, Berland L, Glöckner H, Andreiuk B, Rakhshandehroo T, Uslu S, Mishra S, Clark L, Rashidian M. Direct N- or C-Terminal Protein Labeling Via a Sortase-Mediated Swapping Approach. Bioconjug Chem. 2021 Nov. 17; 32 (11): 2397-2406. PMID: 34748323
62. Ohyama B, Nishifuji K, Chan P T, Kawaguchi A, Yamashita T, Ishii N, Hamada T, Dainichi T, Koga H, Tsuruta D, Amagai M, Hashimoto T. Epitope spreading is rarely found in pemphigus vulgaris by large-scale longitudinal study using desmoglein 2-based swapped molecules. J Invest Dermatol. 2012 April; 132 (4): 1158-1168. PMID: 22277941
63. Tsunoda K, Ota T, Aoki M, Yamada T, Nagai T, Nakagawa T, Koyasu S, Nishikawa T, Amagai M. Induction of pemphigus phenotype by a mouse monoclonal antibody against the amino-terminal adhesive interface of desmoglein 3. J Immunol. 2003 Feb. 15; 170 (4): 2170-2178. PMID: 12574390
64. Cho A, Caldara A L, Ran N A, Menne Z, Kauffman R C, Affer M, Llovet A, Norwood C, Scanlan A, Mantus G, Bradley B, Zimmer S, Schmidt T, Hertl M, Payne A S, Feldman R, Kowalczyk A P, Wrammert J. Single-Cell Analysis Suggests that Ongoing Affinity Maturation Drives the Emergence of Pemphigus Vulgaris Autoimmune Disease. Cell Rep. 2019 Jul. 23; 28 (4): 909-922.e6. PMCID: PMC6684256
65. Bruhns P. Properties of mouse and human IgG receptors and their contribution to disease models. Blood. 2012 Jun. 14; 119 (24): 5640-5649. PMID: 22535666
66. Vidarsson G, Dekkers G, Rispens T. IgG subclasses and allotypes: from structure to effector functions. Front Immunol. 2014; 5:520. PMCID: PMC4202688
67. Yu J, Song Y, Tian W. How to select IgG subclasses in developing anti-tumor therapeutic antibodies. J Hematol Oncol. 2020 May 5; 13 (1): 45. PMCID: PMC7201658
68. Ishikawa H, Li K, Tamai K, Sawamura D, Uitto J. Cloning of the mouse desmoglein 3 gene (Dsg3): interspecies conservation within the cadherin superfamily. Exp Dermatol. 2000 August; 9 (4): 229-239. PMID: 10949543
69. Culton D A, McCray S K, Park M, Roberts J C, Li N, Zedek D C, Anhalt G J, Cowley D O, Liu Z, Diaz L A. Mucosal pemphigus vulgaris anti-Dsg3 IgG is pathogenic to the oral mucosa of humanized Dsg3 mice. J Invest Dermatol. 2015 June; 135 (6): 1590-1597. PMCID: PMC4430403
70. Bosshart H, Heinzelmann M. THP-1 cells as a model for human monocytes. Ann Transl Med. 2016 November; 4 (21): 438. PMCID: PMC5124613
71. Pollmann R, Walter E, Schmidt T, Waschke J, Hertl M, Mobs C, Eming R. Identification of Autoreactive B Cell Subpopulations in Peripheral Blood of Autoimmune Patients With Pemphigus Vulgaris. Front Immunol. 2019; 10:1375. PMCID: PMC6587433
72. Amagai M, Ikeda S, Shimizu H, Iizuka H, Hanada K, Aiba S, Kaneko F, Izaki S, Tamaki K, Ikezawa Z, Takigawa M, Seishima M, Tanaka T, Miyachi Y, Katayama I, Horiguchi Y, Miyagawa S, Furukawa F, Iwatsuki K, Hide M, Tokura Y, Furue M, Hashimoto T, Ihn H, Fujiwara S, Nishikawa T, Ogawa H, Kitajima Y, Hashimoto K, Pemphigus Study Group. A randomized double-blind trial of intravenous immunoglobulin for pemphigus. J Am Acad Dermatol. 2009 April; 60 (4): 595-603. PMID: 19293008
73. Li N, Zhao M, Hilario-Vargas J, Prisayanh P, Warren S, Diaz L A, Roopenian D C, Liu Z. Complete FcRn dependence for intravenous Ig therapy in autoimmune skin blistering diseases. J Clin Invest. 2005 December; 115 (12): 3440-3450. PMCID: PMC1280965
74. Paquin Proulx D, Aubin E, Lemieux R, Bazin R. Inhibition of B cell-mediated antigen presentation by intravenous immunoglobulins (IVIg). Clin Immunol. 2010 June; 135 (3): 422-429. PMID: 20138586
75. Baker K, Rath T, Pyzik M, Blumberg R S. The Role of FcRn in Antigen Presentation. Front Immunol. 2014; 5:408. PMCID: PMC4145246
76. Nimmerjahn F, Ravetch J V. The antiinflammatory activity of IgG: the intravenous IgG paradox. J Exp Med. 2007 Jan. 22; 204 (1): 11-15. PMCID: PMC2118416
77. Mazzi G, Raineri A, Zanolli F A, Da Ponte C, De Roia D, Santarossa L, Guerra R, Orazi B M. Plasmapheresis therapy in pemphigus vulgaris and bullous pemphigoid. Transfus Apher Sci. 2003 February; 28 (1): 13-18. PMID: 12620264
78. Turner M S, Sutton D, Sauder D N. The use of plasmapheresis and immunosuppression in the treatment of pemphigus vulgaris. J Am Acad Dermatol. 2000 December; 43 (6): 1058-1064. PMID: 11100023
79. Kasperkiewicz M, Shimanovich I, Meier M, Schumacher N, Westermann L, Kramer J, Zillikens D, Schmidt E. Treatment of severe pemphigus with a combination of immunoadsorption, rituximab, pulsed dexamethasone and azathioprine/mycophenolate mofetil: a pilot study of 23 patients. Br J Dermatol. 2012 January; 166 (1): 154-160. PMID: 21910700
80. Behzad M, Mobs C, Kneisel A, Möller M, Hoyer J, Hertl M, Eming R. Combined treatment with immunoadsorption and rituximab leads to fast and prolonged clinical remission in difficult-to-treat pemphigus vulgaris. Br J Dermatol. 2012 April; 166 (4): 844-852. PMID: 22092243
81. Laurent S A, Hoffmann F S, Kuhn P-H, Cheng Q, Chu Y, Schmidt-Supprian M, Hauck S M, Schuh E, Krumbholz M, Rübsamen H, Wanngren J, Khademi M, Olsson T, Alexander T, Hiepe F, Pfister H-W, Weber F, Jenne D, Wekerle H, Hohlfeld R, Lichtenthaler S F, Meinl E. Y-Secretase directly sheds the survival receptor BCMA from plasma cells. Nat Commun. 2015 Jun. 11; 6:7333. PMCID: PMC4490565
82. Ridgway J B, Presta L G, Carter P. “Knobs-into-holes” engineering of antibody CH3 domains for heavy chain heterodimerization. Protein Eng. 1996 July; 9 (7): 617-621. PMID: 8844834
83. Wu J, Edberg J C, Redecha P B, Bansal V, Guyre P M, Coleman K, Salmon J E, Kimberly R P. A novel polymorphism of FcgammaRIIIa (CD16) alters receptor function and predisposes to autoimmune disease. J Clin Invest. 1997 Sep. 1; 100 (5): 1059-1070. PMCID: PMC508280
84. Dall'Ozzo S, Tartas S, Paintaud G, Cartron G, Colombat P, Bardos P, Watier H, Thibault G. Rituximab-dependent cytotoxicity by natural killer cells: influence of FCGR3A polymorphism on the concentration-effect relationship. Cancer Res. 2004 Jul. 1; 64 (13): 4664-4669. PMID: 15231679
85. Anolik J H, Campbell D, Felgar R E, Young F, Sanz I, Rosenblatt J, Looney R J. The relationship of FcgammaRIIIa genotype to degree of B cell depletion by rituximab in the treatment of systemic lupus erythematosus. Arthritis Rheum. 2003 February; 48 (2): 455-459. PMID: 12571855
86. Kantarjian H, Stein A, Gökbuget N, Fielding A K, Schuh A C, Ribera J-M, Wei A, Dombret H, Foà R, Bassan R, Arslan Ö, Sanz M A, Bergeron J, Demirkan F, Lech-Maranda E, Rambaldi A, Thomas X, Horst H-A, Brüggemann M, Klapper W, Wood B L, Fleishman A, Nagorsen D, Holland C, Zimmerman Z, Topp M S. Blinatumomab versus Chemotherapy for Advanced Acute Lymphoblastic Leukemia. N Engl J Med. 2017 Mar. 2; 376 (9): 836-847. PMCID: PMC5881572
87. Horn L A, Ciavattone N G, Atkinson R, Woldergerima N, Wolf J, Clements V K, Sinha P, Poudel M, Ostrand-Rosenberg S. CD3×PDL1 bi-specific T cell engager (BiTE) simultaneously activates T cells and NKT cells, kills PDL1+ tumor cells, and extends the survival of tumor-bearing humanized mice. Oncotarget. 2017 Aug. 29; 8 (35): 57964-57980. PMCID: PMC5601626
88. Speck T, Heidbuechel J P W, Veinalde R, Jaeger D, von Kalle C, Ball C R, Ungerechts G, Engeland C E. Targeted BiTE Expression by an Oncolytic Vector Augments Therapeutic Efficacy Against Solid Tumors. Clin Cancer Res. 2018 May 1; 24 (9): 2128-2137. PMID: 29437789
89. Ortho Multicenter Transplant Study Group. A randomized clinical trial of OKT3 monoclonal antibody for acute rejection of cadaveric renal transplants. N Engl J Med. 1985 Aug. 8; 313 (6): 337-342. PMID: 2861567
90. Kujawski M, Li L, Bhattacharya S, Wong P, Lee W-H, Williams L, Li H, Chea J, Poku K, Bowles N, Vaidehi N, Yazaki P, Shively J E. Generation of dual specific bivalent BiTEs (dbBI specific T-cell engaging antibodies) for cellular immunotherapy. BMC Cancer. 2019 Sep. 5; 19 (1): 882. PMCID: PMC6727398
91. Tsunoda K, Ota T, Aoki M, Yamada T, Nagai T, Nakagawa T, Koyasu S, Nishikawa T, Amagai M. Induction of pemphigus phenotype by a mouse monoclonal antibody against the amino-terminal adhesive interface of desmoglein 3. J Immunol. 2003 Feb. 15; 170 (4): 2170-2178. PMID: 12574390
92. Boggon T J, Murray J, Chappuis-Flament S, Wong E, Gumbiner B M, Shapiro L. C-cadherin ectodomain structure and implications for cell adhesion mechanisms. Science. 2002 May 17; 296 (5571): 1308-1313. PMID: 11964443
93. Harrison O J, Brasch J, Lasso G, Katsamba P S, Ahlsen G, Honig B, Shapiro L. Structural basis of adhesive binding by desmocollins and desmogleins. Proc Natl Acad Sci USA. 2016 Jun. 28; 113 (26): 7160-7165.
94. Cho M J, Lo A S Y, Mao X, Nagler A R, Ellebrecht C T, Mukherjee E M, Hammers C M, Choi E-J, Sharma P M, Uduman M, Li H, Rux A H, Farber S A, Rubin C B, Kleinstein S H, Sachais B S, Posner M R, Cavacini L A, Payne A S. Shared VH1-46 gene usage by pemphigus vulgaris autoantibodies indicates common humoral immune responses among patients. Nat Commun. 2014 Jun. 19; 5:4167. PMCID: PMC4120239
95. Takae Y, Nishikawa T, Amagai M. Pemphigus mouse model as a tool to evaluate various immunosuppressive therapies. Exp Dermatol. 2009 March; 18 (3): 252-260. PMID:
96. Aoki-Ota M, Tsunoda K, Ota T, Iwasaki T, Koyasu S, Amagai M, Nishikawa T. A mouse model of pemphigus vulgaris by adoptive transfer of naive splenocytes from desmoglein 3 knockout mice. Br J Dermatol. 2004 August; 151 (2): 346-354. PMID: 15327541
97. Lotti R, Atene C G, Marconi A, Di Rocco G, Reggiani Bonetti L, Zanocco Marani T, Pincelli C. Development of a Desmocollin-3 Active Mouse Model Recapitulating Human Atypical Pemphigus. Front Immunol. 2019; 10:1387. PMCID: PMC6593104
98. Amagai M, Tsunoda K, Suzuki H, Nishifuji K, Koyasu S, Nishikawa T. Use of autoantigen-knockout mice in developing an active autoimmune disease model for pemphigus. J Clin Invest. 2000 March; 105 (5): 625-631. PMCID: PMC292455
99. Courtney A H, Bennett N R, Zwick D B, Hudon J, Kiessling L L. Synthetic antigens reveal dynamics of BCR endocytosis during inhibitory signaling. ACS Chem Biol. 2014 Jan. 17; 9 (1): 202-210. PMCID: PMC3947029
100. Shimanovich I, Baumann T, Schmidt E, Zillikens D, Hammers C M. Long-term outcomes of rituximab therapy in pemphigus. J Eur Acad Dermatol Venereol. 2020 December; 34 (12): 2884-2889. PMID: 32367562
101. Bito T, Nishikawa R, Hatakeyama M, Kikusawa A, Kanki H, Nagai H, Sarayama Y, Ikeda T, Yoshizaki H, Seto H, Adachi A, Horikawa T, Oka M, Nishigori C. Influence of neutralizing antibodies to adalimumab and infliximab on the treatment of psoriasis. Br J Dermatol. 2014 April; 170 (4): 922-929. PMID: 24329764
102. Schmidt E, Hennig K, Mengede C, Zillikens D, Kromminga A. Immunogenicity of rituximab in patients with severe pemphigus. Clin Immunol. 2009 September; 132 (3): 334-341. PMID: 19502112
103. Boyer-Suavet S, Andreani M, Lateb M, Savenkoff B, Brglez V, Benzaken S, Bernard G, Nachman P H, Esnault V, Seitz-Polski B. Neutralizing Anti-Rituximab Antibodies and Relapse in Membranous Nephropathy Treated With Rituximab. Front Immunol. 2019; 10:3069. PMCID: PMC6970431
104. Krishna M, Nadler S G. Immunogenicity to Biotherapeutics—The Role of Anti-drug Immune Complexes. Front Immunol. 2016; 7:21. PMCID: PMC4735944
105. Maselli D J, Singh H, Diaz J, Peters J I. Efficacy of omalizumab in asthmatic patients with IgE levels above 700 IU/mL: a retrospective study. Ann Allergy Asthma Immunol. 2013 June; 110 (6): 457-461. PMID: 23706716
106. Bongard N, Le-Trilling V T K, Malyshkina A, Rückborn M, Wohlgemuth K, Wensing I, Windmann S, Dittmer U, Trilling M, Bayer W. Immunization with a murine cytomegalovirus based vector encoding retrovirus envelope confers strong protection from Friend retrovirus challenge infection. PLOS Pathog. 2019 September; 15 (9): e1008043. PMCID: PMC6786657
107. Korns Johnson D, Homann D. Accelerated and improved quantification of lymphocytic choriomeningitis virus (LCMV) titers by flow cytometry. PLOS One. 2012; 7 (5): e37337. PMCID: PMC3355137
108. Yamada D H, Elsaesser H, Lux A, Timmerman J M, Morrison S L, de la Torre J C, Nimmerjahn F, Brooks D G. Suppression of Fcγ-receptor-mediated antibody effector function during persistent viral infection. Immunity. 2015 Feb. 17; 42 (2): 379-390. PMCID: PMC4334737
109. Pratumchai I, Zak J, Huang Z, Min B, Oldstone M B A, Teijaro J R. B cell-derived IL-27 promotes control of persistent LCMV infection. Proc Natl Acad Sci USA. 2022 Jan. 18; 119 (3): e2116741119. PMCID: PMC8784116
110. Shen H, Whitmire J K, Fan X, Shedlock D J, Kaech S M, Ahmed R. A specific role for B cells in the generation of CD8 T cell memory by recombinant Listeria monocytogenes. J Immunol. 2003 Feb. 1; 170 (3): 1443-1451. PMID: 12538706
111. Lee C-C, Kung J T. Marginal zone B cell is a major source of Il-10 in Listeria monocytogenes susceptibility. J Immunol. 2012 Oct. 1; 189 (7): 3319-3327. PMID: 22933629
112. Misumi I, Whitmire J K. B cell depletion curtails CD4+ T cell memory and reduces protection against disseminating virus infection. J Immunol. 2014 Feb. 15; 192 (4): 1597-1608. PMCID: PMC3925510
113. Mandrup O A, Ong S C, Lykkemark S, Dinesen A, Rudnik-Jansen I, Dagnæs-Hansen N F, Andersen J T, Alvarez-Vallina L, Howard K A. Programmable half-life and anti-tumour effects of bispecific T-cell engager-albumin fusions with tuned FcRn affinity. Commun Biol. 2021 Mar. 8; 4 (1): 310. PMCID: PMC7940400
114. Saxena A, Wu D. Advances in Therapeutic Fc Engineering-Modulation of IgG-Associated Effector Functions and Serum Half-life. Front Immunol. 2016; 7:580. PMCID: PMC5149539

### Example 8

Animal models of pemphigus disease in mice are known (discussed herein). In experiments, the therapeutics disclosed herein have been administered to these mice to determine their effects on disease in the mice (e.g., Example 7). The data from these experiments show that the therapeutics are effective. Additional studies like this are described in this example.

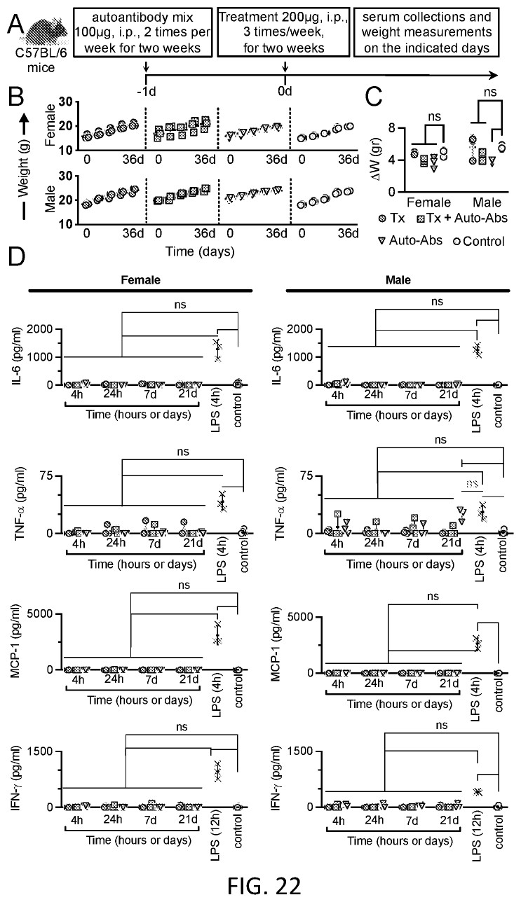

FIG. 22 Panels A-D demonstrate efficacy, as well as that treatment does not result in a cytokine storm nor toxicity, in immunocompetent mice, even in presence of autoantibodies. The schedule for injecting mice with treatment (mDsg3-EC14-mFc) and/or the mix of autoantibodies (AK23, AK19, AK18) is shown in Panel A. In Panels B & C, treatment-recipient mice, with or without autoantibodies, are shown to continue to gain normal weight with no observed toxicities (n=6 for each group; 3 male and 3 female). Panel D shows ELISA analyses that demonstrated no cytokine storm in any of the three groups that received either the treatment alone, autoantibodies, or both autoantibodies and the treatment (using ELISA MAX™ Standard Set Mouse (BioLegend)). Sera from 6 mice that did not receive anything were used for healthy controls in each gender. Samples from 3 mice that received 2 mg/kg of lipopolysaccharide (LPS) were used to mimic a cytokine storm in each gender. Unpaired t-test was used to test for significant differences between groups. (ns, P>0.05; *, P≤0.05; **, P≤0.01; ***, P≤0.001; ****, P≤0.0001).

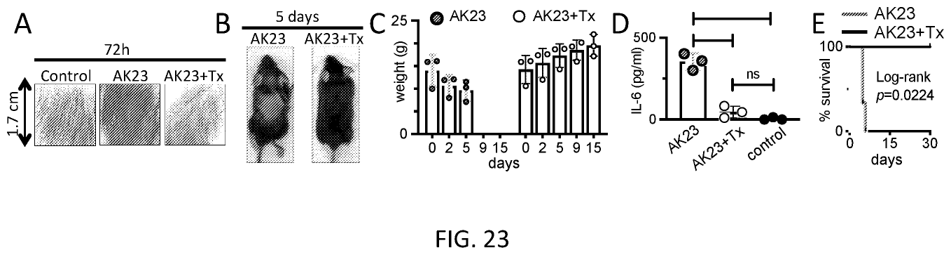

FIG. 23 Panels A-E shows that the treatment (mDsg3-EC1-4-mFc) inhibits pathogenic effects of AK23 antibody. All animals received AK23 (12 mg/kg; subcutaneous); the AK23+treatment cohort received mDsg3-mFc 1 h later (15 mg/kg, i.p). Panels A and B show the AK23 group demonstrated significant hair-loss by tape-stripping performed 72 h later (Panel A), and showed severe PV phenotype (i.e. hair-loss and mucosal erosions (not shown)) at day 5 (Panel B); however, the “AK23+Treatment” group was similar to the control healthy mice (Panels A and B); data representative of n=3 for each cohort. Panel C shows that the AK23 cohort showed weight-loss; however, weight-gain was normal in the mice that received AK23 and the treatment. Panel D shows ELISA analyses of IL6 on sera collected at 72 h and demonstrated high levels of the cytokine in the AK23 cohort, but it was similar to healthy control in the “AK23+treatment” group. Panel E shows survival analyses of the experiment. All AK23 mice developed severe PV and had to be euthanized <7 days. All the treatment mice behaved normally and are alive (n=3 for each cohort).

### EQUIVALENTS

Those skilled in the art will recognize, or be able to ascertain, using no more than routine experimentation, numerous equivalents to the specific substances and procedures described herein. Such equivalents are considered to be within the scope of this invention.

1. A fusion protein comprising an extracellular (EC) domain or part of an EC domain of desmoglein 3 (Dsg3), or an EC domain or part of an EC domain of desmoglein 1 (Dsg1); and an isolated immunoglobulin Fc region or a fragment thereof.

2. The fusion protein of claim 1, wherein the fusion protein comprises any one of SEQ ID NOs: 1-4 or fragments thereof.

3. The fusion protein of claim 1, comprising an amino acid sequence that is at least 80% identical to SEQ ID Nos: 1-4 or 29-32.

4. The fusion protein of claim 1, wherein the fusion protein targets one or more B cells.

5. (canceled)

6. The fusion protein of claim 1, wherein the Fc region comprises an amino acid sequence at least 80% identical to an Fc region in SEQ ID NOs: 1-4 or 29-32.

7. The fusion protein of claim 1, wherein the Fc region comprises an IgG Fc region.

8. The fusion protein of claim 1, wherein the EC domain comprises any one of SEQ ID NOs. 8-28.

9. The fusion protein of claim 8, wherein the EC domain comprises EC1, EC2, EC4, EC5, or combinations thereof.

10. The fusion protein of claim 1, further comprising a therapeutic moiety, an imaging moiety, a capturing moiety, or a combination thereof.

11-14. (canceled)

15. A pharmaceutical composition comprising the fusion protein according to claim 1 and a pharmaceutically acceptable carrier.

16-17. (canceled)

18. A method of treating an autoimmune disease, the method comprising administering to a subject a therapeutically effective amount of the fusion protein of claim 1, wherein the subject is suffering from the autoimmune disease.

19. The method according to claim 18, wherein the disease is selected from the group consisting of an autoimmune blistering disease, lupus, scleroderma, Goodpasture's disease, Graves' disease, and immune-mediated vasculitis.

20. The method according to claim 19, wherein the autoimmune blistering disease comprises pemphigus vulgaris, bullous pemphigoid, mucous membrane pemphigoid, epidermolysis bullosa acquisita, or linear IgA bullous dermatosis.

21-22. (canceled)

23. A nucleic acid encoding the fusion protein according to claim 1.

24-48. (canceled)

49. A fusion protein, comprising:
an autoreactive B cell epitope from an extracellular portion of a desmoglein or desmocollin protein; and
an Fc portion of an antibody that can bind to an Fc-gamma receptor (FcγR), Fc-alpha receptor (FcαR) or Fc-epsilon receptor (FcεR).

50-51. (canceled)

52. The fusion protein of claim 49, wherein the fusion protein comprises at least two B cell epitopes from an extracellular portion of a desmoglein or desmocollin protein.

53. The fusion protein of claim 52, wherein the fusion protein comprises:
a first polypeptide having a first autoreactive antigen and a first C_{H}3 domain from an antibody heavy chain; and
a second polypeptide having a second autoreactive antigen and a second C_{H}3 domain from an antibody heavy chain;
wherein a disulfide bond connects the first polypeptide and the second polypeptide.

54-56. (canceled)

57. A fusion protein, comprising:
an autoreactive B cell epitope from an extracellular portion of a desmoglein or desmocollin protein; and
an antibody that binds an effector cell.

58. The fusion protein of claim 57, wherein the effector cell comprises a T cell.

59. (canceled)

60. The fusion protein of claim 57, wherein the antibody comprises an anti-CD8 (cytotoxic T cells) or an anti-CD4 (helper T cells) antibody.

61. The fusion protein of claim 57, wherein the antibody comprises an antibody specific for macrophages or natural killer (NK) cells.

62-77. (canceled)

78. A method of depleting autoreactive B cells, the method comprising administering to a subject a therapeutically effective amount of the fusion protein of claim 49.

79-87. (canceled)
